(19) United States
(12) Patent Application Publication
(10) Pub. No.: US 2016/0297830 A1
(43) Pub. Date: Oct. 13, 2016

(54) NOVEL COMPOUNDS USEFUL FOR THE TREATMENT OF BACTERIAL INFECTIOUS DISEASES

(71) Applicant: GALAPAGOS NV, Mechelen (BE)

(72) Inventors: Philip John DUDFIELD, Zagreb (HR); John LOWTHER, Champigny-sur-Marne (FR); Carole Annie Josette DELACHAUME, Romainville (FR); Renaud Henri Marcel LÉPINE, Romainville (FR); Romain Vincent Raphaël ROTH DIT BETTONI, Romainville (FR); Julie Marie-Hèléne Marthe GUILLAUME, Romainville (FR); Amber Paula Marcella THYS, Vossem (BE); Anne Catherine BARON, Lyon (FR); Sophie Lucienne Jeanne CANOVA, Lyon (FR); Robert WITZIG, Basel (CH); Julien Georges Pierre-Olivier DOYON, Beerse (BE); Mathieu Paul TOUMI, Draveil (FR); Friedrich Georg HANSSKE, Weinheim (DE)

(21) Appl. No.: 14/915,638

(22) PCT Filed: Sep. 2, 2013
(86) PCT No.: PCT/EP2013/068054

§ 371 Date: Feb. 29, 2016

(51) Int. Cl. C07D 493/08; A61K 31/422; A61K 31/381; A61K 31/4155; A61K 31/4025; A61K 31/365

(52) U.S. Cl. CPC C07D 493/08; A61K 31/4025; A61K 31/365; A61K 31/381; A61K 31/4155; A61K 31/422

(57) ABSTRACT

Compounds are disclosed that have a formula represented by the following:

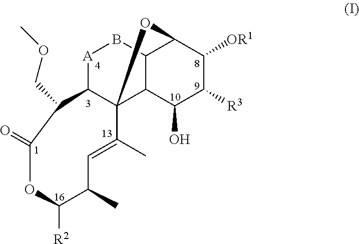

wherein A, B, R¹, R² and R³ are as defined herein.

Novel compounds of the invention may be prepared as a pharmaceutical composition, and may be useful in the treatment of infectious diseases, in particular bacterial infectious diseases. The compounds may be active against a specific enzyme in the bacterial DNA replicative process, DNA polymerase IIIE.

### FIELD OF THE INVENTION

The present invention relates to novel compounds that are useful in the treatment of infectious diseases, in particular those causing significant morbidity in human medicine. In one aspect, the compounds are active against a specific enzyme in the bacterial DNA replicative process, DNA polymerase IIIE. The present invention also provides methods for the production of these novel compounds, pharmaceutical compositions comprising these compounds, and methods for the prevention and/or treatment of bacterial infectious diseases by administering the compound of the invention.

### BACKGROUND OF THE INVENTION

The emergence of resistance to antibiotics in bacteria causing infections in the clinic presents a global and urgent medical threat. The Infectious Disease Society of America highlighted the most problematic species, the so called ESKAPE bacteria: Enterococcus faecium, Staphylococcus aureus, Klebsiella pneumoniae, Acinetobacter baumanii, Pseudomonas aeruginosa, and Enterobacter (Clinical Infectious Diseases 2009; 48:1-12) in which there is a danger that existing therapies will soon no longer be effective. Of particular concern is the methicillin resistant Staphylococcus aureus (MRSA) which was reported to be responsible for more deaths in US hospitals than HIV/AIDS and tuberculosis combined. Once firmly established in hospitals worldwide, MRSA has now emerged as a significant community-acquired pathogen. Community-acquired MRSA infections are increasing, and may now involve persons without risk factors predisposing for acquisition, including children.

The urgency is recognized by governments and health organizations alike, including the WHO which since 2004 has listed infections due to resistant bacteria at the top of their preliminary ranking of pharmaceutical gaps, (Kaplan W, Laing R. Priority Medicines for Europe and the World. Geneva: Department of Essential Drugs and Medicines Policy, World Health Organization, 2004).

The discovery and development of new agents with novel mechanisms of action is clearly imperative if delivery of therapies is to keep pace with the growing problem. Herein we describe a newly identified series of compounds, which are believed to show a novel mode of action designed to address the clinical needs of today and the future.

In the clinical setting, it is a clear advantage for a new antibiotic to have pharmacokinetic properties which give potential for oral administration as well allowing infrequent dosing, ideally once a day. Both properties increase patient convenience and compliance for treatment in the community, and also in terms of follow-on treatment at home after an initial hospital parenteral regimen.

Whilst many naturally occurring compounds have potent anti-bacterial activity, this is often coupled with poor pharmacokinetic and related properties, resulting in compounds which do not exhibit therapeutically relevant in vivo activity, despite showing potent activity against whole bacterial cells when tested in vitro microbroth susceptibility.

One such family of natural polyketide antibiotics are nargenicins and branimycin which have a tricyclic structure with either a 10- or a 9-membered lactone ring and which contain a unique ether bridge. In 1977, the nargenicin family of antibiotics was isolated by Pfizer and Upjohn scientists after aerobic fermentation of Nocardia argentinensis ATCC 31306. One of these compounds, nargenicin A1 was subsequently patented and its structure elucidated (see W. D. Celmer, et al J. Am. Chem. Soc. 102 (1980) 4203-4209). Although in vitro antibacterial activity was shown, it was restricted to Gram-positive methicillin resistant bacteria Staphylococcus aureus (MRSA). It has also been shown by Kim S H, et al. (Biochemical Pharmacology 77 (2009) 1694-1701) that nargenicin A1 induces cell differentiation and that it can be used as a possible treatment for neoplastic diseases. In 1998, branimycin was isolated from Actinomycete GW 60/1571. In vitro biological tests have shown it is active against Bacillus subtilis, Escherichia coli, Staphylococcus aureus and Streptomyces viridochromogenes.

The present invention provides novel compounds which exhibit in vivo activity in animal models of infection, in particular when dosed orally. In a specific aspect, they exhibit improved activity compared to the naturally occurring molecules. These compounds may also exhibit improved properties, including improved pharmacokinetic properties (e.g. solubility, bioavailability, stability and/or, exposure). In community settings it is desirable for drugs to be active via the oral route. The compounds of the invention are efficacious in treating infections in vivo, particularly via the oral route and therefore potentially provide clinically effective treatment in mammals.

### SUMMARY OF THE INVENTION

The present invention relates to novel compounds that may be useful for the treatment of bacterial infectious diseases. The present invention also provides methods for the preparation of the compounds of the invention, intermediates for their preparation, pharmaceutical compositions comprising a compound of the invention and methods for treating bacterial infectious diseases by administering a compound of the invention.

In a first aspect the invention relates to a compound according to Formula (I):

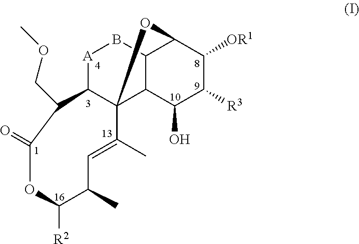

wherein,
A and B together form a bivalent radical —CH═CH— or —CH₂—CH₂—;
R¹ is H or —C(═O)—R⁴,
   R⁴ is a 5-membered heteroaryl containing 1 or 2 heteroatoms selected from O, S and N, optionally substituted by one or more CH₃, halogen, or CN;
R² is H or CR^{2a}R^{2b}R^{2c},
   R^{2a} is selected from H, OH, and OCH₃,
   R^{2b} is H or CH₃, or
   R^{2a} and R^{2b} together form oxo or ═N—OR⁵, wherein R⁵ is H, CH₃, or —C(═O)CH₃,
   R^{2c} is CH₂—O—CH₃; and
R³ is CH₃ or CH₂—O—CH₃;
with the proviso that when A and B together form a bivalent radical —CH═CH—, R² is CR^{2a}R^{2b}R^{2c}, where R^{2a} is OH, R^{2b} is H and R^{2c} is CH₂—O—CH₃, and R³ is CH₂—O—CH₃, then R¹ is —C(═O)—R⁴.

The present invention also relates to pharmaceutical compositions comprising a compound of the invention.

In a further aspect, the present invention provides pharmaceutical compositions comprising a compound of the invention, and a pharmaceutical carrier, excipient or diluent.

In another aspect the invention relates to a compound of the invention for use in therapy.

In another aspect, the invention relates to the use of a compound of the invention in the manufacture of a medicament for the treatment of bacterial infectious disease.

In a further aspect the invention relates to methods of treating a bacterial infectious disease selected from amongst those listed herein, and particularly, where said bacterial infectious disease is caused by Gram negative and/or Gram positive bacteria, which method comprises administering a therapeutically effective amount of the compound of the invention to a subject in need thereof.

In a further aspect, the present invention relates to a compound of the invention for use in the treatment of a bacterial infectious disease by inhibiting DNA polymerase IIIE activity in the bacteria.

Accordingly, it is a principal object of this invention to provide the compound of the invention, which can treat or prevent bacterial infections. In a specific aspect, it is an object of this invention to inhibit DNA polymerase IIIE activity in the bacteria and thus prevent or treat bacterial infectious diseases.

A still further object of this invention is to provide pharmaceutical compositions that may be used in the treatment or prevention of bacterial infectious diseases, by inhibiting DNA polymerase IIIE activity in the bacteria.

In additional aspect, this invention provides methods for preparation of a compound of the invention, with representative synthetic protocols and pathways disclosed herein.

Other objects and advantages will become apparent to those skilled in the art from a consideration of the ensuing detailed description.

It will be appreciated that compounds of the invention may be metabolized to yield biologically active metabolites.

### DETAILED DESCRIPTION OF THE INVENTION

### Definitions

It will be understood that the present invention covers all combinations of aspects, suitable, convenient and preferred groups described herein.

When describing the invention, which may include compounds, pharmaceutical compositions containing such compounds and methods of using such compounds and compositions, the following terms, if present, have the following meanings unless otherwise indicated. Unless otherwise stated, the term ‘substituted’ is to be defined as set out below. It should be further understood that the terms ‘groups’ and ‘radicals’ can be considered interchangeable when used herein.

When ranges are referred to herein, for example but without limitation, C₁₋₆ alkyl, the citation of a range should be considered a representation of each member of said range.

The articles ‘a’ and ‘an’ may be used herein to refer to one or to more than one (i.e. at least one) of the grammatical objects of the article. By way of example ‘an analogue’ means one analogue or more than one analogue.

‘Alkyl’ refers to a straight or branched aliphatic hydrocarbon having the specified number of carbon atoms. Particular alkyl groups have 1 to 6 carbon atoms or 1 to 4 carbon atoms. Branched means that one or more alkyl groups such as methyl, ethyl or propyl is attached to a linear alkyl chain. Particular alkyl groups are methyl, ethyl, n-propyl, isopropyl, n-butyl, tert-butyl, sec-butyl, n-pentyl, n-hexyl, and 1,2-dimethylbutyl. Further particular alkyl groups have between 1 and 4 carbon atoms.

‘Alkoxy’ refers to the group —OR′ where R^{a} is alkyl with the number of carbon atoms specified. Particularly where R^{a} is C₁-C₆ alkyl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

The term ‘alkenyl’ as used herein as a group or a part of a group refers to a straight or branched hydrocarbon chain containing the specified number of carbon atoms and containing at least one double bond. For example, the term “C₂₋₆ alkenyl” means a straight or branched alkenyl containing at least 2, and at most 6, carbon atoms and containing at least one double bond. Similarly, the term “C₃₋₆ alkenyl” means a straight or branched alkenyl containing at least 3, and at most 6, carbon atoms and containing at least one double bond. Examples of “alkenyl” as used herein include, but are not limited to, ethenyl, 2-propenyl, 3-butenyl, 2-butenyl, 2-pentenyl, 3-pentenyl, 3-methyl-2-butenyl, 3-methyl but-2-enyl, 3-hexenyl and 1,1-dimethylbut-2-enyl.

‘Amino’ refers to the radical —NH₂.

The term ‘amino protecting group’ refers to a substituent on an functional amino group which prevent undesired reactions and degradations during synthetic procedures, and which may be selectively removed after certain synthetic step. Examples of ‘amino protecting group’ include: acyl type protecting groups (e.g. formyl, trifluoroacetyl and acetyl), aromatic urethane type protecting groups (e.g. benzyloxycarbonyl (CBz) and substituted Cbz and 9-fluorenylmethoxycarbonyl (Fmoc)), aliphatic urethane protecting groups (e.g. t-butyloxycarbonyl (Boc), isopropyloxycarbonyl and cyclohexyloxycarbonyl) and alkyl type protecting groups (e.g. benzyl, trityl, chlorotrityl).

‘Aryl’ refers to a monovalent aromatic hydrocarbon group derived by the removal of one hydrogen atom from a single carbon atom of a parent aromatic ring system. In particular aryl refers to an aromatic ring structure, mono-cyclic or poly-cyclic that includes the number of ring members specified. Particular aryl groups have from 6 to 10 ring members. Where the aryl group is a monocyclic ring system it preferentially contains 6 carbon atoms. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentalene, pentalene, pentaphene, perylene, phenalene, phenanthrene, picene, pleiadene, pyrene, pyranthrene, rubicene, triphenylene and trinaphthalene. Particularly aryl groups include phenyl, naphthyl, indenyl, and tetrahydronaphthyl.

The term ‘comprise’, and variations such as ‘comprises’ and ‘comprising’, throughout the specification and the claims which follow, unless the context requires otherwise, will be understood to imply the inclusion of a stated integer or step or group of integers but not to the exclusion of any other integer or step or group of integers or steps.

The term ‘compound(s) of the invention’ or ‘compound(s) according to the invention’, and equivalent expressions includes both compounds of the Formula(e) as herein described, specifically compounds of Formula (I) (whether in solvated or unsolvated form), or its pharmaceutically acceptable salts (whether in solvated or unsolvated form). Suitably, said expression includes the pharmaceutically acceptable salts, and solvates (e.g. hydrates) thereof. The compounds of the invention may possess one or more asymmetric centers; such compounds can therefore be produced as individual (R)- or (S)-stereoisomers or as mixtures thereof. Where stereochemistry is not defined in the relevant Formula(e), then the term compounds of the invention includes enantiomers and diastereoisomers of these compounds.

‘Cyano’ refers to the radical —CN.

The term ‘halogen’ or ‘halo’ refers to fluoro (F), chloro (Cl), bromo (Br) and iodo (I). Particularly the halo group is chloro.

‘Hetero’ when used to describe a compound or a group present on a compound means that one or two carbon atoms in the compound or group have been replaced by a nitrogen, oxygen, or sulfur heteroatom.

‘Heteroaryl’ or ‘heteroaromatic’ means a mono-cyclic aromatic ring structure that includes one or two heteroatoms independently selected from oxygen, nitrogen and sulphur and the number of ring members specified. Particular heteroaryl group has five ring members. In one embodiment, the heteroaryl ring contains at least one ring nitrogen atom. The nitrogen atoms in the heteroaryl rings can be basic, as in the case of an imidazole, or essentially non-basic as in the case of a pyrrole nitrogen. Examples of five membered monocyclic heteroaryl groups include but are not limited to pyrrole, furan, thiophene, imidazole, oxazole, isoxazole, thiazole, isothiazole, pyrazole and triazole groups. Particular heteroaryl groups are those derived from pyrrole, furan, thiophene, pyrazole, oxazole and isoxazole. Specifically heteroaryl group is derived from pyrrole.

Examples of representative heteroaryls include the following:

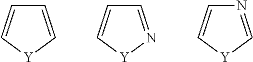

wherein each Y is selected from N, O and S.

‘Hydroxy’ refers to the radical —OH.

The term ‘hydroxy protecting group’ refers to a substituent on an functional hydroxyl group which prevent undesired reactions and degradations during synthetic procedures, and which may be selectively removed after certain synthetic step. Examples of ‘hydroxy protecting group’ include: ester and ether hydroxyl protecting group. Examples of ester hydroxyl protecting group include: formyl, —OC(O)C₁₋₄alkyl such as acetyl (Ac or —C(O)CH₃), methoxyacetyl, chloroacetyl, dichloroacetyl, trichloroacetyl, trifluoroacetyl, triphenylmethoxyacetyl, phenoxyacetyl, benzoylformyl, benzoyl (Bz or —C(O)C₆H₅), benzyloxycarbonyl (Cbz or —C(O)—O—CH₂C₆H₅), methoxycarbonyl, tert-butoxycarbonyl, isopropoxycarbonyl, diphenylmethoxycarbonyl or 2-(trimethylsilyl)ethoxycarbonyl and the like. Examples of ether hydroxyl protecting group include: alkyl silyl groups such as trimethylsilyl (TMS), tert-butyldimethylsilyl, triethylsilyl, triisopropylsilyi and the like. Examples of suitable ‘hydroxy protecting group’ include; —OC(O)C₁₋₄alkyl such as acetyl (Ac or —C(O)CH₃), benzoyl (Bz), benzyloxycarbonyl (Cbz) and trimethylsilyl (TMS). Suitably, ‘hydroxy protecting group’ is: triethylsilyl or acetyl (Ac or —C(O)CH₃). Conveniently, ‘hydroxy protecting group’ is: triethylsilyl.

As used herein, term ‘substituted with one or more’ refers to one to four substituents. In one embodiment it refers to one to three substituents. In further embodiment it refers to one or two substituents. In a yet further embodiment it refers to one substituent.

‘Substituted’ refers to a group in which one or more hydrogen atoms are each independently replaced with the same or different substituent(s).

‘Pharmaceutically acceptable’ means approved or approvable by a regulatory agency of the Federal or a state government or the corresponding agency in countries other than the United States, or that is listed in the U.S. Pharmacopoeia or other generally recognized pharmacopoeia for use in mammals, and more particularly, in humans.

‘Pharmaceutically acceptable salt’ refers to a salt of a compound that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term ‘pharmaceutically acceptable cation’ refers to an acceptable cationic counter-ion of an acidic functional group. Such cations are exemplified by sodium, potassium, calcium, magnesium, ammonium, tetraalkylammonium cations, and the like.

‘Pharmaceutically acceptable vehicle’ refers to a diluent, adjuvant, excipient or carrier with which a compound of the invention is administered.

The term ‘prodrug’ as used herein refers to compounds, including derivatives of the compounds of the invention, which have metabolically cleavable groups and are converted within the body e.g. by solvolysis or under physiological conditions into the compounds of the invention which are pharmaceutically active in vivo. Pharmaceutically acceptable prodrugs are described in: Bundgard, H. Design of Prodrugs, pp. 7-9, 21-24, Elsevier, Amsterdam 1985, T. Higuchi and V. Stella, “Prodrugs as Novel Delivery Systems”, Vol. 14 of the A.C.S. Symposium Series; Edward B. Roche, ed., “Bioreversible Carriers in Drug Design”, American Pharmaceutical Association and Pergamon Press, 1987; and in D. Fleisher, S. Ramon and H. Barbra “Improved oral drug delivery: solubility limitations overcome by the use of prodrugs”, Advanced Drug Delivery Reviews (1996) 19(2) 115-130. Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Particularly useful are the C₁-C₈ alkyl, C₂-C₈ alkenyl, aryl, and C₇-C₁₂ arylalkyl esters of the compounds of the invention.

‘Solvate’ refers to forms of the compound that are associated with a solvent, usually by a solvolysis reaction. This physical association includes hydrogen bonding. Conventional solvents include water, ethanol, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. ‘Solvate’ encompasses both solution-phase and isolable solvates. Representative solvates include hydrates, ethanolates and methanolates.

The term ‘isotopic variant’ refers to a compound that contains unnatural proportions of isotopes at one or more of the atoms that constitute such compound. For example, an ‘isotopic variant’ of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy. All isotopic variants of the compounds provided herein, radioactive or not, are intended to be encompassed within the scope of the invention.

The term ‘isomer(s)’ refers to compounds that have the same molecular formula but differ in the nature or sequence of bonding of their atoms or the arrangement of their atoms in space. Isomers that differ in the arrangement of their atoms in space are termed ‘stereoisomers’.

‘Diastereomers’ are stereoisomers that are not mirror images of one another and those that are non-superimposable mirror images of each other are termed ‘enantiomers’. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a ‘racemic mixture’.

‘Tautomers’ refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

‘Subject’ refers to an animal, in particular a mammal and more particular to a human or a domestic animal serving as a model for a disease (for example guinea pigs, mice, rats, gerbils, cats, rabbits, dogs, monkeys, chimpanzees or like). Specifically the subject is a human. The terms ‘patient’ and ‘subject’ are used interchangeably herein.

‘Therapeutically effective amount’ means the amount of a compound of the invention that, when administered to a subject for treating an infection, is sufficient to effect such treatment for the infection. For example, but without limitation, the treatment of an invention may involve decreasing the number of bacteria causing said infection in the patient. The “therapeutically effective amount” can vary depending on the compound, the infection and its severity, and the age, weight, physical condition, responsiveness etc., of the subject to be treated and will ultimately be at the discretion of the attendant physician.

‘Preventing’ or ‘prevention’ refers to a reduction in risk of acquiring or developing an infection (i.e., causing at least one of the clinical symptoms of the infection not to develop in a subject that may be exposed to an infection-causing agent, or predisposed to the infection in advance of infection onset).

The term ‘prophylaxis’ is related to ‘prevention’, and refers to a measure or procedure the purpose of which is to prevent, rather than to treat or cure an infection. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

‘Treating’ or ‘treatment’ of any infection refers, in one embodiment, to ameliorating the infection (i.e., arresting the infection or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the infection, either physically, (e.g., stabilization of a discernible symptom), physiologically, (e.g., stabilization of a physical parameter), or both. In a further embodiment, ‘treating’ or ‘treatment’ relates to decreasing the bacterial load associated with the infection.

As used herein, the term ‘bacterial infectious diseases’ refers to diseases caused by bacterial infection and includes systemic infections (bacteremia and sepsis) and/or infections of any organ or tissue of the body. These organs or tissue include, without limitation, skeletal muscle, skin, bloodstream, kidneys, heart, lung and bone. These infections may be caused by Gram-positive or Gram-negative bacteria as described below. Specifically, said bacterial infectious disease is caused by Gram-positive bacteria.

As used herein, the term ‘Gram-negative bacteria’ refers to bacteria which do not retain crystal violet dye in the Gram staining protocol and includes, but is not limited to, bacteria in the Genus Enterobacteriacae, including Escherichia spp. (including E. coli), Klebsiella spp., Enterobacter spp., Citrobacter spp., Serratia spp., Proteus spp., Providencia spp., Salmonella spp., Shigella spp., the genus Pseudomonas (including P. aeruginosa) and species such as Moraxella spp. (including M. catarrhalis), Haemophilus spp. and Neisseria spp.

As used herein, the term ‘Gram-positive bacteria’ refers to bacteria which are stained dark blue or violet by Grain staining and includes, but is not limited to, methicillin-susceptible and methicillin-resistant staphylococci (including Staphylococcus aureus, S. epidermidis, S. haemolyticus, S. hominis, S. saprophyticus, and coagulase-negative staphylococci), glycopeptideintermediary-susceptible S. aureus (GISA), penicillin-susceptible and penicillin-resistant streptococci (including Streptococcus pneumoniae, S. pyogenes, S. agalactiae, S. avium, S. bovis, S. lactis, S. sanguis and Streptococci Group C, Streptococci Group G and viridans streptococci), enterococci (including vancomycin-susceptible and vancomycin-resistant strains such as Enterococcus faecalis and E. faecium), Clostridium difficile, Listeria monocytogenes, Corynebacterium jeikeium, Chlamydia spp (including C. pneumoniae) and Mycobacterium tuberculosis.

### The Compounds

The present invention is based on the identification that the compounds of the invention may be useful for the treatment of bacterial infectious diseases, particularly in mammals. The present invention also provides methods for the preparation of the compounds of the invention, the intermediates for their preparation, pharmaceutical compositions comprising a compound of the invention and methods for the treatment of bacterial infectious diseases in mammals by administering the compound of the invention.

In a specific embodiment the compounds of the invention are inhibitors of DNA Polymerase IIIE

In a particular embodiment, as DNA polymerase IIIE represents a novel target for antibacterial agents and the compound of the invention is an unexploited chemical class, the compound of the invention is active against bacterial strains which exhibit resistance to established classes of antibiotics. Therefore, in one embodiment the present invention provides the compound of the invention for use in the treatment of bacterial infectious diseases caused by strains resistant to established antibiotic classes. In a specific embodiment the present invention provides the compound of the invention for use in the treatment of bacterial infectious diseases caused by strains resistant to aminoglycosides, carbapenems, cephalosporins, glycopeptides, lincosamides, lipopeptide, macrolides, monobactams, nitrofurans, oxazolidonones, penicillins, polypeptides, quinolones, sulfonamides, fusidic acid, pseudomonic acids, rifamycins, lipoglycopeptides, novobiocin, and/or tetracyclines (e.g. glycylcyclines).

Accordingly, in a first aspect the invention relates to a compound of the invention according to Formula (I):

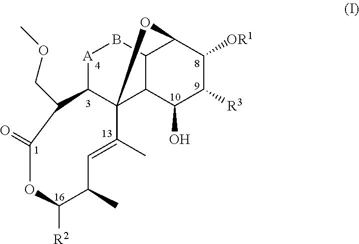

wherein,
A and B together form a bivalent radical —CH═CH— or —CH₂—CH₂—;
R¹ is H or —C(═O)—R⁴,
   R⁴ is a 5-membered heteroaryl containing 1 or 2 heteroatoms selected from O, S and N, optionally substituted by one or more CH₃, halogen, or CN;
R² is H or CR^{2a}R^{2b}R^{2c},
   R^{2a} is selected from H, OH, and OCH₃,
   R^{2b} is H or CH₃, or
   R^{2a} and R^{2b} together form oxo or ═N—OR⁵, wherein R⁵ is H, CH₃, or —C(═O)CH₃,
   R^{2c} is CH₂—O—CH₃; and
R³ is CH₃ or CH₂—O—CH₃;
with the proviso that when A and B together form a bivalent radical —CH═CH—, R² is CR^{2a}R^{2b}R^{2c}, where R^{2a} is OH, R^{2b} is H and R^{2c} is CH₂—O—CH₃, and R³ is CH₂—O—CH₃, then R¹ is —C(═O)—R⁴.

In one embodiment, the compound of the invention is according to Formula I, wherein R¹ is —C(═O)R⁴.

In one embodiment, the compound of the invention is according to Formula I, wherein R¹ is H.

In one embodiment, the compound of the invention is according to Formula I, wherein A and B together form a bivalent radical —CH═CH— and R¹ is as described in any one of the embodiments above.

In one embodiment, the compound of the invention is according to Formula I, wherein A and B together form a bivalent radical —CH₂—CH₂— and R¹ is as described in any one of the embodiments above.

In one embodiment, the compound of the invention is according to Formula I, wherein A and B are as described in any one of the embodiments above, R¹ is —C(═O)R⁴, and R⁴ is selected from:

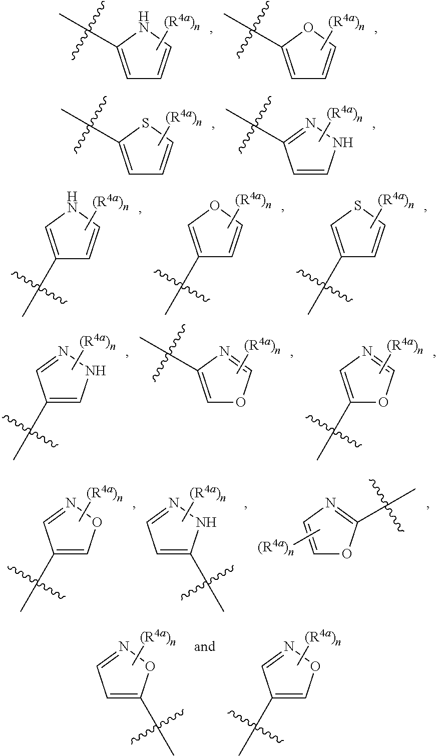

wherein R^{4a} is selected from H, CN, CH₃ and halogen, and integer n is 1 or 2.

In one embodiment, the compound of the invention is according to Formula I, wherein A and B are as described in any one of the embodiments above, R¹ is —C(═O)R⁴, and R⁴ is selected from:

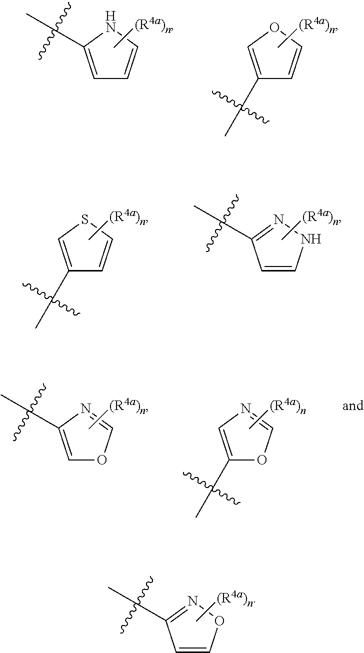

In one embodiment, the compound of the invention is according to Formula I, wherein A and B are as described in any one of the embodiments above, R¹ is —C(═O)R⁴, and R⁴ is selected from:

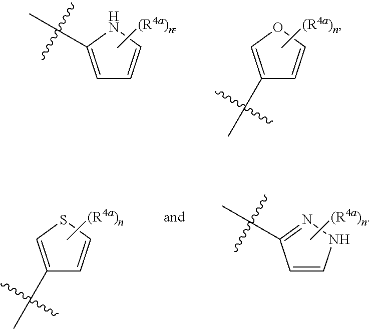

In one embodiment, the compound of the invention is according to Formula I, wherein A and B are as described in any one of the embodiments above, R¹ is —C(═O)R⁴, and R⁴ is selected from:

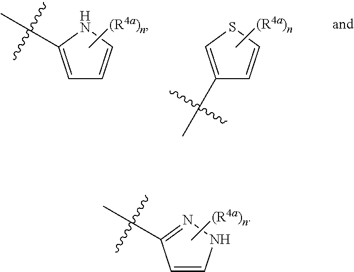

In one embodiment, the compound of the invention is according to Formula I, wherein A and B are as described in any one of the embodiments above, R¹ is —C(═O)R⁴, and R⁴ is selected from:

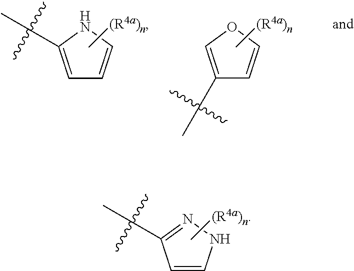

In one embodiment, the compound of the invention is according to Formula I, wherein A and B are as described in any one of the embodiments above, R¹ is —C(═O)R⁴, and R⁴ is:

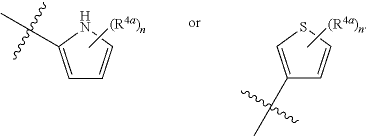

In one embodiment, the compound of the invention is according to Formula I, wherein A and B are as described in any one of the embodiments above, R¹ is —C(═O)R⁴, and R⁴ is:

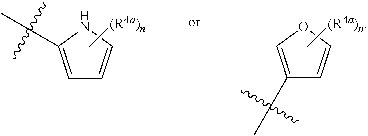

In one embodiment, the compound of the invention is according to Formula I, wherein A and B are as described in any one of the embodiments above, R¹ is —C(═O)R⁴, and R⁴ is:

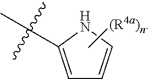

In one embodiment, the compound of the invention is according to Formula I, wherein A, B and R⁴ are as described in any one of the embodiments above, R¹ is —C(═O)R⁴, and when R⁴ is

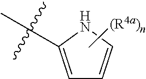

it is selected from:

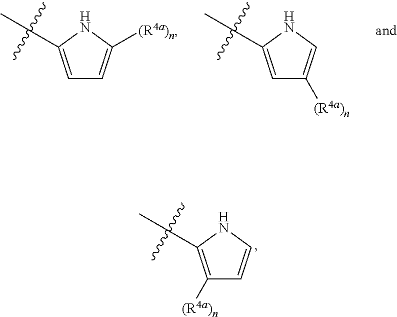

wherein n is 1.

In one embodiment, the compound of the invention is according to Formula I, wherein R¹ is —C(═O)R⁴, A, B and R⁴ are as described in any one of the embodiments above, wherein

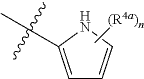

represents

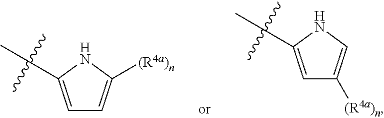

wherein n is 1.

In one embodiment, the compound of the invention is according to Formula I, wherein R¹ is —C(═O)R⁴, A, B and R⁴ are as described in any one of the embodiments above, wherein R^{4a} is selected from H, CN, CH₃ and halogen and integer n is 1.

In one embodiment, the compound of the invention is according to Formula I, wherein R¹ is —C(═O)R⁴, A, B and R⁴ are as described in any one of the embodiments above, wherein R^{4a} is H.

In one embodiment, the compound of the invention is according to Formula I, wherein R¹ is —C(═O)R⁴, A, B and R⁴ are as described in any one of the embodiments above, wherein R^{4a} is halogen, and integer n is 1 or 2. In a particular embodiment R^{4a} is F or Cl, and integer n is 1.

In one embodiment, the compound of the invention is according to Formula I, wherein R¹ is —C(═O)R⁴, A, B and R⁴ are as described in any one of the embodiments above, wherein R^{4a} is H or halogen, and integer n is 1 or 2. In a particular embodiment R^{4a} is F or Cl, and integer n is 1.

In one embodiment, the compound of the invention is according to Formula I, wherein A, B, R¹ and R⁴ are as described in any one of the embodiments above and R² is H.

In one embodiment, the compound of the invention is according to Formula I, wherein A, B, R¹ and R⁴ are as described in any one of the embodiments above and R² is CR^{2a}R^{2b}R^{2c}, wherein R^{2a} is OH, or OCH₃, R^{2b} is H and R^{2c} is CH₂—O—CH₃. In a particular embodiment CR^{2a}R^{2b}R^{2c}, wherein R^{2a} is OH, R^{2b} is H and R^{2c} is CH₂—O—CH₃. In a more particular embodiment CR^{2a}R^{2b}R^{2c} is

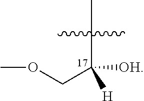 [SMILES: *[C@H](O)COC]

In one embodiment, the compound of the invention is according to Formula I, wherein A, B, R¹ and R⁴ are as described in any one of the embodiments above and R² is CR^{2a}R^{2b}R^{2c}, wherein R^{2a} is OH, R^{2b} is CH₃ and R^{2c} is CH₂—O—CH₃.

In one embodiment, the compound of the invention is according to Formula I, wherein A, B, R¹ and R⁴ are as described in any one of the embodiments above and R² is CR^{2a}R^{2b}R^{2c}, wherein R^{2a} and R^{2b} are H and R^{2c} is CH₂—O—CH₃.

In one embodiment, the compound of the invention is according to Formula I, wherein A, B, R¹ and R⁴ are as described in any one of the embodiments above and R² is CR^{2a}R^{2b}R^{2c}, wherein R^{2a} and R^{2b} together form oxo and R^{2c} is CH₂—O—CH₃.

In one embodiment, the compound of the invention is according to Formula I, wherein A, B, R¹ and R⁴ are as described in any one of the embodiments above and R² is CR^{2a}R^{2b}R^{2c}, wherein R^{2a} and R^{2b} together form ═N—OR⁵ and R^{2c} is CH₂—O—CH₃, wherein R⁵ is H, CH₃, or —C(═O)CH₃. In a particular embodiment R⁵ is H or CH₃. In another particular embodiment R⁵ is H. In a further particular embodiment R⁵ is CH₃.

In one embodiment, the compound of the invention is according to Formula I, wherein A, B, R¹, R² and R⁴ are as described in any one of the embodiments above and R³ is CH₃.

In one embodiment, the compound of the invention is according to Formula I, wherein A, B, R¹, R² and R⁴ are as described in any one of the embodiments above and R³ is CH₂—O—CH₃.

In one embodiment, the compound of the invention is according to Formula I, wherein A and B together form a bivalent radical —CH═CH—, R¹ is H, R² is CR^{2a}R^{2b}R^{2c}, wherein R^{2a} is OH, R^{2b} is H and R^{2c} is CH₂—O—CH₃, and R³ is CH₃.

In one embodiment, the compound of the invention is selected amongst the following compounds:
baleomycin,
8-O-1H-pyrrole-2′-carbonylbranimycin,
8-O-1H-pyrrole-2′-carbonylbaleomycin,
17-deoxy-8-O-1H-pyrrole-2′-carbonylbranimycin,
8-O-furane-3′-carbonylbranimycin,
8-O-5-methyl-1H-pyrrole-2′-carbonylbranimycin,
8-O-furane-3′-carbonylbaleomycin,
8-O-oxazole-4′-carbonylbranimycin,
8-O-thiophene-3′-carbonylbranimycin,
8-O-1H-pyrazole-3′-carbonylbranimycin,
8-O-2-methylfurane-3′-carbonylbranimycin,
8-O-4-methyl-1H-pyrrole-2′-carbonylbranimycin,
8-O-isoxazole-3′-carbonylbranimycin,
8-O-oxazole-5′-carbonylbranimycin,
4,5-dihydro-branimycin,
4,5-dihydro-8-O-1H-pyrrole-2′-carbonylbranimycin,
17-deoxybranimycin,
17-O-methyl-8-O-1H-pyrrole-2′-carbonylbranimycin,
8-O-4-fluoro-1H-pyrrole-2′-carbonylbranimycin,
18-deoxy-18-oxo-8-O-1H-pyrrole-2′-carbonylbranimycin,
17,18-dinor-branimycin,
17,18-dinor-8-O-1H-pyrrole-2′-carbonybranimycin,
8-O-4-fluoro-1H-pyrrole-2′-carbonylbaleomycin,
17-methyl-8-O-1H-pyrrole-2′-carbonylbranimycin,
18-deoxy-18-oximino-8-O-1H-pyrrole-2′-carbonylbranimycin,
8-O-4-cyano-1H-pyrrole-2′-carbonylbranimycin,
18-deoxy-18-methoximino-8-O-1H-pyrrole-2′-carbonylbranimycin,
8-O-4-chloro-1H-pyrrole-2′-carbonylbranimycin,
18-acetylimino-18-deoxy-8-O-1H-pyrrole-2′-carbonylbranimycin,
8-O-4-chloro-1H-pyrrole-2′-carbonylbaleomycin,
8-O-4,5-dichloro-1H-pyrrole-2′-carbonylbranimycin,
8-O-4-bromo-1H-pyrrole-2′-carbonylbranimycin,
8-O-5-Bromo-4-Chloro-1H-pyrrole-2′-carbonylbranimycin,
8-O-4,5-Dibromo-1H-pyrrole-2′-carbonylbranimycin,
8-O-4-Bromo-5-Chloro-1H-pyrrole-2′-carbonylbranimycin,
8-O-4,5-Dibromofurane-2′-carbonylbranimycin,
8-O-4-Bromo-5-Fluoro-1H-pyrrole-2′-carbonylbranimycin,
8-O-4,5-Difluoro-1H-pyrrole-2′-carbonylbranimycin,
8-O-5-Methyl-1H-Pyrazole-3′-carbonylbranimycin
18-Deoxy-18-oximino-8-O-4-Fluoro-1H-pyrrole-2′-carbonylbranimycin,
18-Deoxy-18-oximino-8-O-4-Chloro-1H-pyrrole-2′-carbonylbranimycin, and
18-Acetylimino-18-deoxy-8-O-4-Chloro-1H-pyrrole-2′-carbonylbranimycin.

In one particular embodiment, the present invention relates to 8-O-4-fluoro-1H-pyrrole-2′-carbonylbranimycin.

In one particular embodiment, the present invention relates to 8-O-1H-pyrrole-2′-carbonylbranimycin.

In one particular embodiment, the compound of the invention is not 8-O-1H-pyrrole-2′-carbonylbranimycin.

In one embodiment the compound of the invention is not an isotopic variant.

In one aspect a compound of the invention according to any one of the embodiments herein described is a free base.

In one aspect a compound of the invention according to any one of the embodiments herein described is a salt. In one particular embodiment, the present invention relates to a salt of 8-O-4-fluoro-1H-pyrrole-2′-carbonylbranimycin. In an alternative embodiment, the present invention relates to a salt of 8-O-1H-pyrrole-2′-carbonylbranimycin.

In one aspect a compound of the invention according to any one of the embodiments herein described is a pharmaceutically acceptable salt. In one particular embodiment, the present invention relates to a pharmaceutically acceptable salt of 8-O-4-fluoro-1H-pyrrole-2′-carbonylbranimycin. In an alternative embodiment, the present invention relates to a pharmaceutically acceptable salt of 8-O-1H-pyrrole-2′-carbonylbranimycin.

In one aspect a compound of the invention according to any one of the embodiments herein described is a solvate of the compound.

In one aspect a compound of the invention according to any one of the embodiments herein described is a solvate of a salt of a compound, in particular a solvate of a pharmaceutically acceptable salt.

Similarly, reference to intermediates, whether or not they themselves are claimed, is meant to embrace their salts, and solvates, where the context so permits.

With regard to stereoisomers, the compounds of the invention have more than one asymmetric carbon atom. In the general formula(e) as drawn, the solid wedge shaped bond indicates that the bond is above the plane of the paper. The broken bond indicates that the bond is below the plane of the paper.

It will be appreciated that the substituents on the compounds of the invention may also have one or more asymmetric carbon atoms. Thus, the compounds of the invention may occur as individual enantiomers or diastereomers. All such isomeric forms are included within the present invention, including mixtures thereof.

Where a compound of the invention contains an alkenyl group, cis (Z) and trans (E) isomerism may also occur. The present invention includes the individual stereoisomers of the compound of the invention and, where appropriate, the individual tautomeric forms thereof, together with mixtures thereof.

Separation of diastereoisomers or cis and trans isomers may be achieved by conventional techniques, e.g. by fractional crystallisation, chromatography or HPLC. A stereoisomeric mixture of the agent may also be prepared from a corresponding optically pure intermediate or by resolution, such as by HPLC, of the corresponding mixture using a suitable chiral support or by fractional crystallisation of the diastereoisomeric salts formed by reaction of the corresponding mixture with a suitable optically active acid or base, as appropriate.

In a further aspect, the present invention provides a method for the synthesis of a compound according to Formula I where R¹ is —C(═O)—R⁴, said method comprising:
(a) reacting a compound according to Formula II below, with a suitable hydroxyl protection agent R^{P1}—X to protect position C¹⁷ hydroxyl, wherein X is a leaving group and R^{P1} is a suitable protecting group, and
wherein in Formula II, A and B together form a bivalent radical —CH═CH— or —CH₂—CH₂—; and R³ is CH₃ or CH₂—O—CH₃,

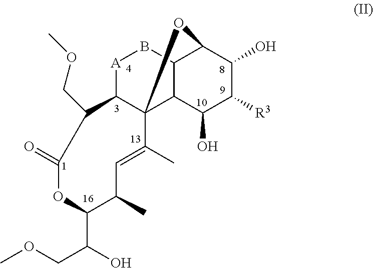

(b) acylating the product of step (a) (Intermediate A) at the C⁸-position with R⁴—COOH, wherein R⁴ is 5-membered heteroaryl containing 1 or 2 heteroatoms selected from O, S and N, optionally substituted by one or more CH₃, halogen, or CN, optionally protected by one or more R^{P3} protecting groups selected from —C(═O)OC₁₋₆ alkyl, —C(═O)C₁₋₆ alkyl, —CH₂-Ph, —Si(C₁₋₄ alkyl)₃, —Si(C₁₋₄ alkyl)(Ph)₂, tetrahydropyranyl, —SO₂-Ph and allyl, wherein said alkyl, and phenyl groups may further be substituted with C₁₋₈ alkyl, C₁₋₄ alkoxy, —Si(C₁₋₆alkyl)₃, NO₂, or halo and
(c) removal of all protecting groups R^{P1} and R^{P3} from the intermediate B obtained in step
(b) above to yield a compound according to Formula I, where R¹ is —C(═O)—R⁴, A and B together form a bivalent radical —CH═CH— or —CH₂—CH₂—, R² is CR^{2a}R^{2b}R^{2c}, where R^{2a} is OH, R^{2b} is H and R^{2c} is —CH₂—O—CH₃, and R³ is CH₃ or CH₂—O—CH₃

In one aspect, in the method described above in step (a) R^{P1}—X is a silyl ether group, particularly TES-Cl, TBDPS-Cl, TBDMS-Cl, or TMS-Cl.

In one further aspect, in the method described above in step (b) R^{P3} is selected from —C(═O)OtBu, —CH₂-Ph, —C(═O)O(CH₂)₂Si(Me)₃, and 4-Me-Ph-SO₂—.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

While specified groups for each embodiment have generally been listed above separately, a compound of the invention may be one for which one or more variables (R groups and/or integers) is selected from one or more embodiments according to any of the Formula(e) listed above. Therefore, the present invention is intended to include all combinations of variables from any of the disclosed embodiments within its scope.

Alternatively, the exclusion of one or more of the specified variables from a group or an embodiment of said group, or combinations thereof is also contemplated by the present invention.

### PHARMACEUTICAL COMPOSITIONS

When employed as a pharmaceutical, a compound of the invention is typically administered in the form of a pharmaceutical composition. Such compositions can be prepared in a manner well known in the pharmaceutical art and comprise at least one active compound. Generally, a compound of the invention is administered in a pharmaceutically effective amount. The amount of the compound actually administered will typically be determined by a physician, in the light of the relevant circumstances, including the infection to be treated, the chosen route of administration, the actual compound administered, the age, weight, and response of the individual patient, the severity of the patient's symptoms, and the like.

The pharmaceutical compositions of the invention can be administered by a variety of routes including oral, rectal, transdermal, subcutaneous, intra-articular, intravenous, intramuscular, and intranasal. Depending on the intended route of delivery, the compound of this invention is preferably formulated as either injectable, including intravenous, or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient, vehicle or carrier. Typical unit dosage forms include prefilled, premeasured ampoules or syringes of the liquid compositions or pills, tablets, capsules or the like in the case of solid compositions. In such compositions, the compound of the invention is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

A compound of the invention can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17^{th} edition, 1985, Mack Publishing Company, Easton, Pa., which is incorporated herein by reference.

A compound of the invention can also be administered in sustained release forms or from sustained release drug delivery systems. A description of representative sustained release materials can be found in Remington's Pharmaceutical Sciences.

The following formulation examples illustrate representative pharmaceutical compositions that may be prepared in accordance with this invention. The present invention, however, is not limited to the following pharmaceutical compositions.

Moreover, a compound of the present invention useful in the pharmaceutical compositions and treatment methods disclosed herein, is pharmaceutically acceptable as prepared and used. In this aspect of the invention, the pharmaceutical composition may additionally comprise further active ingredients suitable for use in combination with a compound of the invention.

### Formulation 1

### Tablets

A compound of the invention may be admixed as a dry powder with a dry gelatin binder in an approximate 1:2 weight ratio. A minor amount of magnesium stearate may be added as a lubricant. The mixture may be formed into 240-270 mg tablets (80-90 mg of active amide compound per tablet) in a tablet press.

### Formulation 2

### Capsules

A compound of the invention may be admixed as a dry powder with a starch diluent in an approximate 1:1 weight ratio. The mixture may be filled into 250 mg capsules (125 mg of active amide compound per capsule).

### Formulation 3

### Liquid

A compound of the invention (125 mg), may be admixed with sucrose (1.75 g) and Xanthan gum (4 mg) and the resultant mixture may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color may be diluted with water and added with stirring. Sufficient water may then be added with stirring, particularly sufficient water may be added to produce a total volume of 5 mL.

### Formulation 4

### Tablets

A compound of the invention may be admixed as a dry powder with a dry gelatin binder in an approximate 1:2 weight ratio. A minor amount of magnesium stearate may be added as a lubricant. The mixture is formed into 450-900 mg tablets (150-300 mg of active amide compound) in a tablet press.

### Formulation 5

### Injection

A compound of the invention may be dissolved or suspended in a buffered sterile saline injectable aqueous medium to a concentration of approximately 5 mg/mL.

### Formulation 6

### Topical

Stearyl alcohol (250 g) and a white petrolatum (250 g) may be melted at about 75° C. and then a mixture of a compound of the invention (50 g) methylparaben (0.25 g), propylparaben (0.15 g), sodium lauryl sulfate (10 g), and propylene glycol (120 g) dissolved in water (about 370 g) may be added and the resulting mixture may be stirred until it congeals.

### Methods of Treatment

In one aspect, the present invention provides a compound of the invention for use as a medicament.

In a further aspect, the present invention provides a compound of the invention for use in the treatment of bacterial infectious diseases, particularly in mammals. In an alternative embodiment, said bacterial infectious disease is caused by Gram-negative bacteria. In a specific embodiment, said bacterial infectious disease is caused by Gram-positive bacteria. In a further specific embodiment the present invention provides the compound of the invention for use in the treatment of bacterial infectious diseases caused by strains resistant to established antibiotic classes. In a further specific embodiment the present invention provides the compound of the invention for use in the treatment of bacterial infectious diseases caused by strains resistant to aminoglycosides, carbapenems, cephalosporins, glycopeptides, lincosamides, lipopeptide, macrolides, monobactams, nitrofurans, oxazolidonones, penicillins, polypeptides, quinolones, sulfonamides, fusidic acid, pseudomonic acids, rifamycins, lipoglycopeptides, novobiocin, and/or tetracyclines (e.g. glycylcyclines).

In a further aspect, the present invention provides a compound of the invention for use in the manufacture of a medicament for the treatment of bacterial infectious diseases, particularly in mammals. In a particular embodiment, said bacterial infectious disease is caused by Gram-negative bacteria. In a specific embodiment, said bacterial infectious disease is caused by Gram-positive bacteria. In a further specific embodiment the present invention provides a compound of the invention for use in the manufacture of a medicament for the treatment of bacterial infectious diseases caused by strains resistant to established antibiotic classes. In a further specific embodiment, said bacterial infectious diseases are caused by strains resistant to aminoglycosides, carbapenems, cephalosporins, glycopeptides, lincosamides, lipopeptide, macrolides, monobactams, nitrofurans, oxazolidonones, penicillins, polypeptides, quinolones, sulfonamides, fusidic acid, pseudomonic acids, rifamycins, lipoglycopeptides, novobiocin, and/or tetracyclines (e.g. glycylcyclines).

In a further aspect, the present invention provides a method of treating bacterial infectious diseases, particularly in mammals, said method comprising administering a therapeutically effective amount of a compound of the invention, to a patient in need thereof. In a particular embodiment, said bacterial infectious disease is caused by Gram-negative bacteria. In a specific embodiment, said bacterial infectious disease is caused by Gram-positive bacteria. In a further specific embodiment the bacterial infectious diseases are caused by strains resistant to established antibiotic classes. In a further specific embodiment, said bacterial infectious diseases are caused by strains resistant to aminoglycosides, carbapenems, cephalosporins, glycopeptides, lincosamides, lipopeptide, macrolides, monobactams, nitrofurans, oxazolidonones, penicillins, polypeptides, quinolones, sulfonamides, fusidic acid, pseudomonic acids, rifamycins, lipoglycopeptides, novobiocin, and/or tetracyclines (e.g. glycylcyclines).

In a specific embodiment, the present invention provides a compound of the invention for use in the treatment of bacterial infections caused by a Gram-positive bacteria selected from methicillin-susceptible and methicillin-resistant staphylococci (including Staphylococcus aureus, Staphylococcus epidermidis, Staphylococcus haemolyticus, Staphylococcus hominis, Staphylococcus saprophyticus, and coagulase-negative staphylococci), glycopeptides-intermediate susceptible Staphylococcus aureus (GISA), penicillin-susceptible and penicillin-resistant streptococci (including Streptococcus pneumoniae, Streptococcus pyogenes, Streptococcus agalactiae, Streptococcus avium, Streptococcus bovis, Streptococcus lactis, Streptococcus sanguis and Streptococci Group C (GCS), Streptococci Group G (GGS) and viridans streptococci), enterococci (including vancomycin-susceptible and vancomycin-resistant strains such as Enterococcus faecalis and Enterococcus faecium), Clostridium difficile, Listeria monocytogenes, Corynebacterium jeikeium, Chlamydia spp (including C. pneumoniae) and Mycobacterium tuberculosis. In a more specific embodiment the Gram-positive bacteria is Staphylococcus aureus, in particular methicillin-resistant S. aureus (MRSA).

In a specific embodiment, the present invention provides a compound of the invention for use in the manufacture of a medicament for the treatment of bacterial infections caused by a Gram-positive bacteria selected from methicillin-susceptible and methicillin-resistant staphylococci (including Staphylococcus aureus, Staphylococcus epidermidis, Staphylococcus haemolyticus, Staphylococcus hominis, Staphylococcus saprophyticus, and coagulase-negative staphylococci), glycopeptides-intermediate susceptible Staphylococcus aureus (GISA), penicillin-susceptible and penicillin-resistant streptococci (including Streptococcus pneumoniae, Streptococcus pyogenes, Streptococcus agalactiae, Streptococcus avium, Streptococcus bovis, Streptococcus lactis, Streptococcus sanguis and Streptococci Group C (GCS), Streptococci Group G (GGS) and viridans streptococci), enterococci (including vancomycin-susceptible and vancomycin-resistant strains such as Enterococcus faecalis and Enterococcus faecium), Clostridium difficile, Listeria monocytogenes, Corynebacterium jeikeium, Chlamydia spp (including C. pneumoniae) and Mycobacterium tuberculosis. In a more specific embodiment the Gram-positive bacteria is Staphylococcus aureus, in particular methicillin-resistant S. aureus (MRSA).

In a specific embodiment, the present invention provides a method of treating bacterial infections caused by a Gram-positive bacteria selected from methicillin-susceptible and methicillin-resistant staphylococci (including Staphylococcus aureus, Staphylococcus epidermidis, Staphylococcus haemolyticus, Staphylococcus hominis, Staphylococcus saprophyticus, and coagulase-negative staphylococci), glycopeptides-intermediate susceptible Staphylococcus aureus (GISA), penicillin-susceptible and penicillin-resistant streptococci (including Streptococcus pneumoniae, Streptococcus pyogenes, Streptococcus agalactiae, Streptococcus avium, Streptococcus bovis, Streptococcus lactis, Streptococcus sanguis and Streptococci Group C (GCS), Streptococci Group G (GGS) and viridans streptococci), enterococci (including vancomycin-susceptible and vancomycin-resistant strains such as Enterococcus faecalis and Enterococcus faecium), Clostridium difficile, Listeria monocytogenes, Corynebacterium jeikeium, Chlamydia spp (including C. pneumoniae) and Mycobacterium tuberculosis, said method comprising administering a therapeutically effective amount of the compound of the invention, to a patient in need thereof. In a more specific embodiment the Gram-positive bacteria is Staphylococcus aureus, in particular methicillin-resistant S. aureus (MRSA).

In a specific embodiment, the present invention provides a compound of the invention for use in the treatment of bacterial infections caused by a Gram-negative bacteria selected from bacteria in the Genus Enterobacteriacae, including Escherichia spp. (including Escherichia coli), Klebsiella spp., Enterobacter spp., Citrobacter spp., Serratia spp., Proteus spp., Providencia spp., Salmonella spp., Shigella spp., the genus Pseudomonas (including P. aeruginosa), Moraxella spp. (including M. catarrhalis), Haemophilus spp and Neisseria spp. In a more specific embodiment the Gram-negative bacteria is in the Genus Enterobacteriacae, including Escherichia spp. (including Escherichia coli), Klebsiella spp., Enterobacter spp., Citrobacter spp., Serratia spp., Proteus spp., Providencia spp., Salmonella spp., Shigella spp., or is P. aeruginosa.

In a specific embodiment, the present invention provides a compound of the invention for use in the manufacture of a medicament for the treatment of bacterial infections caused by a Gram-negative bacteria selected from bacteria in the Genus Enterobacteriacae, including Escherichia spp. (including Escherichia coli), Klebsiella spp., Enterobacter spp., Citrobacter spp., Serratia spp., Proteus spp., Providencia spp., Salmonella spp., Shigella spp., the genus Pseudomonas (including P. aeruginosa), Moraxella spp. (including M. catarrhalis), Haemophilus spp and Neisseria spp. In a more specific embodiment the Gram-negative bacteria is in the Genus Enterobacteriacae, including Escherichia spp. (including Escherichia coli), Klebsiella spp., Enterobacter spp., Citrobacter spp., Serratia spp., Proteus spp., Providencia spp., Salmonella spp., Shigella spp., or is P. aeruginosa.

In a specific embodiment, the present invention provides a method of treating bacterial infections caused by a Gram-negative bacteria selected from bacteria in the Genus Enterobacteriacae, including Escherichia spp. (including Escherichia coli), Klebsiella spp., Enterobacter spp., Citrobacter spp., Serratia spp., Proteus spp., Providencia spp., Salmonella spp., Shigella spp., the genus Pseudomonas (including P. aeruginosa), Moraxella spp. (including M. catarrhalis), Haemophilus spp and Neisseria spp, said method comprising administering a therapeutically effective amount of the compound of the invention, to a patient in need thereof. In a more specific embodiment the Gram-negative bacteria is in the Genus Enterobacteriacae, including Escherichia spp. (including Escherichia coli), Klebsiella spp., Enterobacter spp., Citrobacter spp., Serratia spp., Proteus spp., Providencia spp., Salmonella spp., Shigella spp., or is P. aeruginosa.

In a specific aspect, the present invention provides a compound of the invention for use as a therapeutic agent for the treatment of bacterial infections caused by more than one strain of Gram-positive bacteria, or a bacterial infection caused by both Gram-positive and Gram-negative bacteria. These types of infections include intra-abdominal infections and obstetrical/gynecological infections.

In a specific aspect, the present invention provides a compound of the invention for use in the manufacture of a medicament for the treatment of bacterial infections caused by more than one strain of Gram-positive bacteria, or a bacterial infection caused by both Gram-positive and Gram-negative bacteria. These types of infections include intra-abdominal infections and obstetrical/gynecological infections.

In a specific aspect, the present invention provides a method of treating bacterial infections caused by more than one strain of Gram-positive bacteria, or a bacterial infection caused by both Gram-positive and Gram-negative bacteria, said method comprising administering a therapeutically effective amount of the compound of the invention, to a patient in need thereof. These types of infections include intra-abdominal infections and obstetrical/gynecological infections.

In a specific aspect, the present invention provides a compound of the invention for use as a therapeutic agent for the treatment of endocarditis, nephritis, septic arthritis, intra-abdominal sepsis, bone and joint infections and/or osteomyelitis.

In a specific aspect, the present invention provides a compound of the invention for use in the manufacture of a medicament for the treatment of endocarditis, nephritis, septic arthritis, intra-abdominal sepsis, bone and joint infections and/or osteomyelitis.

In a specific aspect, the present invention provides a method of treating endocarditis, nephritis, septic arthritis, intra-abdominal sepsis, bone and joint infections and/or osteomyelitis, said method comprising administering a therapeutically effective amount of the compound of the invention, to a patient in need thereof.

In a further aspect, the present invention provides a compound of the invention for use as a therapeutic agent for the treatment or prevention of bacterial infections in any organ or tissue in the body. In a specific embodiment, the present invention provides a compound of the invention for use as a therapeutic agent for the treatment or prevention of skin and soft tissue infections, bacteremia, urinary tract infections and sexually transmitted bacterial infections. In a specific embodiment, the present invention provides a compound of the invention for use as a therapeutic agent for the treatment or prevention of community acquired respiratory infections, including, without limitation, otitis media, sinusitis, chronic bronchitis and pneumonia. In a specific embodiment, the present invention provides a compound of the invention for use as a therapeutic agent for the treatment or prevention of blood infections, in particular sepsis and/or septicemia.

In a further aspect, the present invention provides a compound of the invention for use in the manufacture of a medicament for the treatment of bacterial infections in any organ or tissue in the body. In a specific embodiment, the present invention provides a compound of the invention for use in the manufacture of a medicament for the treatment of skin and soft tissue infections, bacteremia, urinary tract infections and sexually transmitted bacterial infections. In a specific embodiment, the present invention provides a compound of the invention for use in the manufacture of a medicament for the treatment of community acquired respiratory infections, including, without limitation, otitis media, sinusitis, chronic bronchitis and pneumonia. In a specific embodiment, the present invention provides a compound of the invention for use in the manufacture of a medicament for the treatment of blood infections, in particular sepsis and/or septicemia.

In a further aspect, the present invention provides a method for the treatment of bacterial infections in any organ or tissue in the body, said method comprising administering a therapeutically effective amount of the compound of the invention, to a patient in need thereof. In a specific embodiment, the present invention provides a method for the treatment of skin and soft tissue infections, bacteremia, urinary tract infections and sexually transmitted bacterial infections, said method comprising administering a therapeutically effective amount of the compound of the invention, to a patient in need thereof. In a specific embodiment, the present invention provides a method for the treatment of community acquired respiratory infections, including, without limitation, otitis media, sinusitis, chronic bronchitis and pneumonia, said method comprising administering a therapeutically effective amount of the compound of the invention, to a patient in need thereof. In a specific embodiment, the present invention provides a method for the treatment or prophylaxis of blood infections, in particular sepsis and/or septicemia, said method comprising administering a therapeutically effective amount of the compound of the invention, to a patient in need thereof.

The present invention provides the compound of the invention for use in the treatment or prevention of bacterial infections by inhibiting DNA polymerase IIIE activity in the bacteria.

The present invention provides the compound of the invention for use in the manufacture of a medicament for use in the treatment or prevention of bacterial infections by inhibiting DNA polymerase IIIE activity in the bacteria.

The present invention provides a method for the treatment or prevention of bacterial infections by inhibiting DNA polymerase IIIE activity in the bacteria, said method comprising administering a therapeutically effective amount of a compound of the invention to a patient in need thereof.

The present invention provides a compound of the invention for use as a therapeutic agent for the treatment or prevention of bacterial infections by inhibiting DNA polymerase IIIE activity in the bacteria. Accordingly, a compound and pharmaceutical compositions of the invention find use as therapeutics for preventing and/or treating bacterial infectious diseases in mammals, including humans.

The present invention provides a method of preventing or treating bacterial infectious diseases, said method comprising administering a therapeutically effective amount of a compound of the invention, to a patient in need thereof.

As a further aspect of the invention there is provided a method of treatment, comprising administering a therapeutically effective amount of a compound of the invention to a patient in need thereof. In specific embodiments, the bacterial infectious disease or bacterial strain to be treated may be selected from the embodiments listed above. Also provided herein is the use of the compound in the manufacture of a medicament for the treatment or prevention of one of the aforementioned bacterial infectious diseases or bacterial strain.

A person of skill in the art will appreciate that the methods and uses described above may also be applied to the use of pharmaceutical compositions comprising the compound of the invention.

A particular regimen of the present method comprises the administration to a subject suffering from a bacterial infectious disease, of an effective amount of a compound of the invention for a period of time sufficient to reduce the level of infection in the subject, and preferably terminate said infection. A special embodiment of the method comprises administering of an effective amount of the compound of the invention to a subject patient suffering from or susceptible to the development of a bacterial infectious disease, for a period of time sufficient to reduce or prevent, respectively, infection of said patient, and preferably terminate, said infection.

Injection dose levels range from about 0.1 mg/kg/h to at least 10 mg/kg/h, all for from about 1 to about 120 h and especially 24 to 96 h. A preloading bolus of from about 0.1 mg/kg to about 10 mg/kg or more may also be administered to achieve adequate steady state levels. The maximum total dose is not expected to exceed about 2 g/day for a 40 to 80 kg human patient.

For the prevention and/or treatment of bacterial infectious diseases, the regimen for treatment will typically last from 1 to 30 days. For the treatment of such infections oral dosing is preferred for patient convenience and tolerance. With oral dosing, one to five and especially two to four and typically three oral doses per day are representative regimens. Alternatively, once a day dosing is preferred for patient convenience. Using these dosing patterns, each dose provides from about 0.01 to about 20 mg/kg of the compound of the invention, with particular doses each providing from about 0.1 to about 10 mg/kg and especially about 1 to about 5 mg/kg. Alternatively, the bacterial infectious disease may be treated via the parenteral route in a hospital based setting.

Transdermal doses are generally selected to provide similar or lower blood levels than are achieved using injection doses.

When used to prevent the onset of a condition, the compound of the invention will be administered to a patient at risk for developing the condition, typically on the advice and under the supervision of a physician, at the dosage levels described above. Patients at risk for developing a particular condition generally include those that have been exposed to a particular bacterial infectious agent, who have a suppressed immune system or those who have been identified by screening to be particularly susceptible to developing the condition, for example, but without limitation, patients diagnosed with cystic fibrosis or patients undergoing invasive surgery.

A compound of the invention can be administered as the sole active agent or it can be administered in combination with other therapeutic agents, including other compounds that demonstrate the same or a similar therapeutic activity, and that are determined to be safe and efficacious for such combined administration. In a specific embodiment, co-administration of two (or more) agents allows for significantly lower doses of each to be used, thereby reducing the side effects seen.

In one embodiment, a compound of the invention is co-administered with another therapeutic agent for the treatment and/or prevention of bacterial infectious diseases; particular agents include but are not limited to antibiotics. In a particular embodiment, the compound of the invention is co-administered with another therapeutic agent for the treatment and/or prevention of infections of any organ of the human body; particular agents include but are not limited to aminoglycosides, carbacephem, carbapenems, cephalosporins, glycopeptides, lincosamides, macrolides, monobactams, nitrofurans, penicillins, polypeptides, quinolones, sulfonamides, tetracyclins, anti-mycobacterial agents, as well as chloramphenicol, fosfomycin, linezolid, metronidazole, mupirocin, rifamycin, thiamphenicol and tinidazole.

In one embodiment, a compound of the invention is co-administered with an additional therapeutic agent for the treatment and/or prevention of bacterial infectious diseases caused by Gram-negative bacteria, wherein said additional therapeutic agent is an efflux pump inhibitor or a membrane permeabilising agent.

By co-administration is included any means of delivering two or more therapeutic-agents to the patient as part of the same treatment regime, as will be apparent to the skilled person. Whilst the two or more agents may be administered simultaneously in a single formulation this is not essential. The agents may be administered in different formulations and at different times. Therefore, in one aspect the present invention provides the co-administration of a compound of the invention with one or more additional therapeutic agents where the active agents are present in the same pharmaceutical composition. Alternatively, the present invention provides the co-administration of a compound of the invention with one or more additional therapeutic agents, where each active agent is administered via a separate pharmaceutical composition.

In a specific embodiment, a compound of the invention may be used in combination with a companion diagnostic test to confirm the presence of one or more of the bacterial strains as described herein.

### General Synthetic Procedures

### General

A compound of the invention can be prepared from readily available starting materials using the following general methods and procedures. It will be appreciated that where typical or preferred process conditions (i.e., reaction temperatures, times, mole ratios of reactants, solvents, pressures, etc.) are given other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Wiley-Blackwell; 4th Revised edition edition (2006), and references cited therein.

A compound of the invention may be prepared from known or commercially available starting materials and reagents by one skilled in the art of organic synthesis.

All reagents were of commercial grade and were used as received without further purification, unless otherwise stated. Commercially available anhydrous solvents were used for reactions conducted under inert atmosphere. Reagent grade solvents were used in all other cases, unless otherwise specified. Column chromatography was performed on silica standard (35-70 μm). Thin layer chromatography was carried out using pre-coated silica gel 60 F-254 plates (thickness 0.25 mm)¹H NMR spectra were recorded on a Bruker Advance 400 NMR spectrometer (400 MHz) or a Bruker Advance 300 NMR spectrometer (300 MHz). Chemical shifts (δ) for ¹H NMR spectra are reported in parts per million (ppm) relative to tetramethylsilane (δ 0.00) or the appropriate residual solvent peak as internal reference. Multiplicities are given as singlet (s), doublet (d), triplet (t), quartet (q), multiplet (m) and broad (br). Electrospray MS spectra were obtained on a Waters platform LC/MS spectrometer. Analytic LCMS: Columns used, Waters Acquity UPLC BEH C18 1.7 μm, 2.1 mm ID×50 mm L or Waters Acquity UPLC BEH C18 1.7 μm, 2.1 mm ID×30 mm L All the methods are using MeCN/H₂O gradients. MeCN and H₂O contain either 0.1% formic acid or NH₃ (10 mM). Preparative LCMS: Column used, Waters XBridge Prep C18 5 μm ODB 30 mm ID×100 mm L. All the methods are using MeOH/H₂O gradients. MeOH and H₂O contain either 0.1% Formic Acid or 0.1% Diethylamine. Microwave heating was performed with a Biotage Initiator. Hydrogenation reaction was performed using H-Cube®, HC-2.SS (SS reaction line version)

The following is a list of abbreviations used in the experimental section:

| | |
|---|---|
| ACN | Acetonitrile |
| AcOH | Acetic acid |
| Ac₂O | Acetic anhydride |
| BAIB | [bis(acetoxy)iodo]benzene |
| Boc | tert-Butyloxy-carbonyl |
| br s | broad singlet |
| cat. | Catalytic amount |
| d | doublet |
| DCM | Dichloromethane |
| DCC | N,N′-Dicyclohexylcarbodiimide |
| DCE | 1,2-Dichloroethene, |
| DIPEA | N,N-Diisopropylethylamine |
| DMAP | 4-Dimethylaminopyridine |
| DMSO | Dimethyl sulfoxide |
| EtOAc | Ethyl acetate |
| Et₂O | Diethyl ether |
| EtOH | Ethanol |
| eq. | equivalents |
| g | gram |
| h | hour |
| HPLC- | High Pressure Liquid |
| MS/CAD | Chromatography-Mass |
| | spectrometry/Charged Aerosol |
| | Detector |
| LCMS | Liquid Chromatography-Mass |
| | Spectrometry |
| m | multiplet |
| MeCN | Acetonitrile |
| MeOH | Methanol |
| MtBE | Methyl-Tert-Butyl-Ether |
| mg | milligram |
| min | minute |
| mL | milliliter |
| μL | microliter |
| MW | Molecular weight calculated |
| MNBA | 2-methyl-6-nitrobenzoic |
| | anhydride |
| MS Mes'd | Measured mass |
| MPLC | Medium Pressure Liquid |
| | Chromatography |
| NMR | Nuclear Magnetic Resonance |
| Pd/C | Palladium on Carbon 10% |
| ppm | part-per-million |
| q | quadruplet |
| Rt | Room temperature |
| s | singlet |
| SEMCl | Chloromethyl 2- |
| | trimethylsilylethyl |
| SM | Starting Material |
| t | triplet |
| TCDI | 1′,1′-thiocarbonyl diimidazole |
| TEOC | 2-Trimethylsilylethyl carbamate |
| TFA | Trifluoroacetic acid |
| THF | Tetrahydrofuran |
| TEMPO | 2,2,6,6-teramethyl-1- |
| | piperidinyloxyl |
| TESCl | Triethylchlorosilane |
| TBAF | Tetra-nbutylammonium fluoride |
| TLC | Thin layer chromatography |
| TsCl | p-Toluenesulfonylchloride |
| TASF | Tris(dimethylamino)sulphonium |
| | difluoro(trimethyl)silicate |
| TBDPS-Cl | tert-Butylchlorodiphenylsilane |
| TBDMS-Cl | tert-Butyldimethylsilyl chloride |
| TMS-Cl | Trimethylsilyl chloride |

The naturally occurring parent molecule branimycin may be prepared by the fermentation procedure described in M Speitling PhD thesis in 2000 or by total synthesis as described in by S. Marchart et. al. in Angew. Chem. Int. Ed (2010) 49 (11): 2050-2053 “Total synthesis of the Antibiotic Branimycin”.

### Synthetic Preparation of Compounds of the Invention

Scheme A below shows the general procedure for the synthesis of selected compounds of the invention. The conversion of the parent molecules into compounds of the invention where A and B together form a bivalent radical —CH═CH— or —CH₂—CH₂—, R¹ is C(═O)—R⁴, R² is as shown and R³ is selected from CH₃ and CH₂—O—CH₃, may be achieved using the following sequence of reactions: a) protection of the 17-hydroxyl using suitable conditions, b) acylation of the 8-position with a R⁴—CO₂H derivative, where the 5-membered heteroaryl group R⁴ is optionally protected, and c) removal of the remaining protecting groups. Exemplary methods are described below, protecting groups suitable are typically silyl ether derivatives such as, but not restricted to, TES, TBDPS, TBDMS, TMS and related, but a person of skill in the art is aware of suitable alternatives.

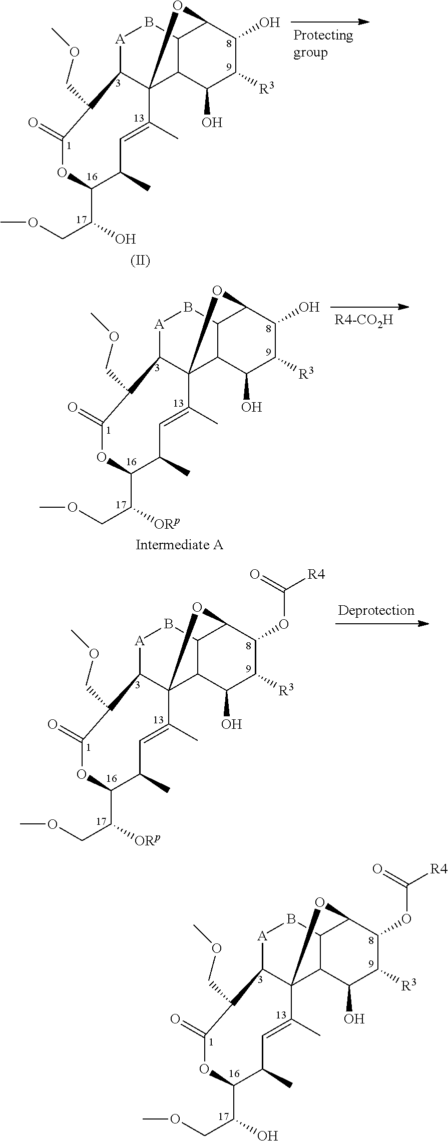

### Method A: General Method for C-17-Hydroxyl Group Protection

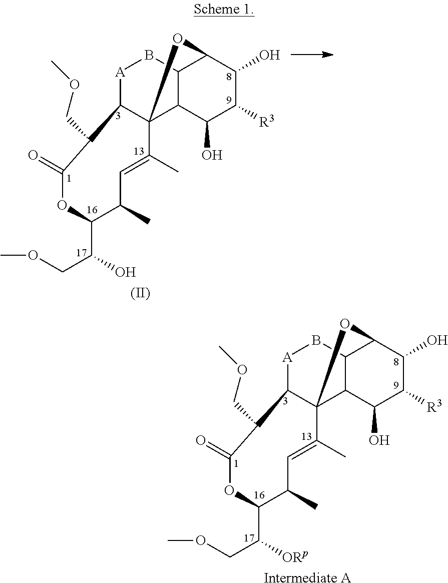

wherein R^{p} is SiEt₃, and A, B, and R³ are as described for Formula (I).

### Intermediate A: Method A General Procedure

A solution of starting compound (II) (1.0 eq.) in a suitable solvent for example a halogenated solvent, e.g. DCM, at 0° C. is treated with imidazole (1.7-2.8 eq.), in the presence of a catalyst (e.g. DMAP (cat.)) and a chlorotrialkylsilane (e.g. TESCl (1.1-1.3 eq.)) and stirred at 0° C. or from 0° C. to room temperature for 1 h to 16 h. A saturated solution of NH₄Cl is added and the mixture diluted with a solvent (e.g. DCM). The aqueous phase is extracted with a solvent (e.g. DCM). The combined organic layers are dried over Na₂SO₄, filtered and concentrated under vacuum to give the desired product, which is used in the next step without further purification.

### Representative Intermediates A

### Intermediate A1: Wherein A and B Together Form Bivalent Radical —CH═CH— and R³ is CH₂OCH₃

A solution of branimycin (2.0 g, 4.15 mmol, 1.0 eq.) in DCM (33 mL) at 0° C. was treated with imidazole (706 mg, 10.4 mmol, 2.5 eq.), DMAP (cat.) and TESCl (871 μL, 5.19 mmol, 1.25 eq.) and stirred at 0° C. for 1 h 15 min. Further amounts of TESCl (8×70 μL) and imidazole (1×140 mg) were added until complete conversion is reached. Then saturated solution of NH₄Cl was added and the mixture diluted with DCM. The aqueous phase was extracted with DCM. The combined organic layers were dried over Na₂SO₄, filtered and concentrated under vacuum to give the desired product, which was used in the next step without further purification.

### Alternative Route to Intermediate A1

To a solution of branimycin (2.12 g, 4.4 mmol, 1 eq.) in dry THF (44 mL), imidazole (750 mg, 11.0 mmol, 2.5 eq.) was added at rt followed by DMAP (cat.). At 0° C. chlorotriethylsilane (965 μL, 5.7 mmol, 1.30 eq.) was added over 15 min and the resulting white suspension was vigorously stirred for 120 min keeping the temperature at ˜0° C. The reaction mixture was diluted with water and EtOAc, the aqueous layer was extracted with EtOAc (250 mL). The combined organic layers were washed with brine, dried over Na₂SO₄ and concentrated. The residue was purified on SiO₂ with (Et₂O/Petroleum ether: 95/5): to give the 17-TES protected Branimycin.

### Intermediate A2: Wherein A and B Together Form Bivalent Radical —CH═CH— and R³ is CH₃

This intermediate was prepared by Method A starting from Compound 1 (baleomycin).

### Intermediate A3: Wherein A and B Together Form Bivalent —CH₂—CH₂— and R³ is CH₂OCH₃

This intermediate was prepared by Method A starting from Compound 15.

### General Methods for the Synthesis-Protection of Pyrroles

To a solution of pyrrole-2-carboxylic acid (2.0 g, 18 mmol, 1.0 eq.) in a (1:1) mixture of dry DMF/THF (60 mL) was added Et₃N (12.6 mL, 90 mmol, 5.0 eq.) followed at rt by benzyl bromide (90 mmol, 10 mL, 5.0 eq.), the resulting suspension was vigorously stirred at rt for one day. The reaction was poured into a mixture of Et₂O and a saturated aqueous solution of NaHCO₃ (50 mL), the aqueous layer was extracted with Et₂O (2×100 mL) and EtOAc (100 mL). The combined organic layers were washed with brine dried over Na₂SO₄, filtered and concentrated. Chromatography on SiO₂ with gradient from pure petroleum ether to EtOAc/petroleum ether: 10/90: gave the protected pyrrole as a solid.

To a solution of the O-benzyl protected pyrrole (3.53 g, 17.5 mmol, 1.0 eq.) in dry THF (40 mL) was added at rt Et₃N (2.7 mL, 19.2 mmol, 1.1 eq.) followed by di-tert-butyl dicarbonate (4.2 g, 19.2 mmol, 1.1 eq.) and DMAP (cat.), the resulting yellow solution was stirred at rt for 60 min and concentrated in vacuo. Chromatography on SiO₂ with (EtOAc/Petroleum ether: 5/95): gave protected pyrrole.

To a solution of pyrrole (1.38 g, 4.6 mmol) in absolute EtOH (25 mL), purged with N₂ was added Pd/C (10% wt, 450 mg) followed by 1,4-cyclohexadiene (2.2 mL, 25 mmol, 5.5 eq.), the resulting solution was vigorously stirred at rt for 120 min. The reaction mixture was filtered through a pall Seitz filter (washed with MeOH ˜200 mL). The filtrate solution was concentrated in vacuo to yield N-Boc pyrrole carboxylic acid.

### N-2-(trimethylsilyl)ethyl-pyrrole-2-carboxylic acid

### a: Introduction of 2-(trimethylsilyl)ethyl protecting group on 1H pyrrole-2-carbaldehyde

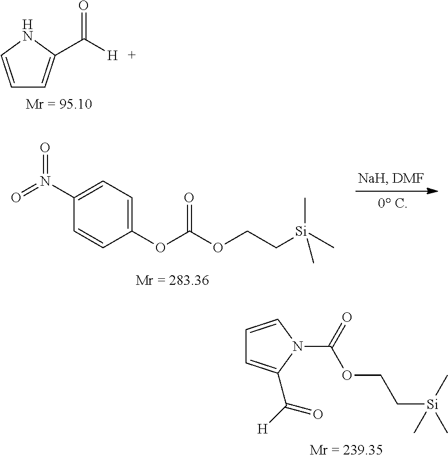 [SMILES: O=Cc1ccc[nH]1 ; C[Si](C)(C)CCOC(=O)Oc1ccc([N+](=O)[O-])cc1 ; C[Si](C)(C)CCOC(=O)n1cccc1C=O]

To a suspension of sodium hydride (164.0 mg, 4.10 mmol, 1.3 eq, 60% in mineral oil) in DMF (2.5 mL), 1H-2-pyrrole-carbaldehyde (300.0 mg, 3.15 mmol, 1.0 eq) was added at 0° C., and the mixture was stirred at the same temperature for 20 minutes. A solution of 4-nitrophenyl-2-trimethylsilyl-ethyl carbonate (1.16 g, 4.10 mmol, 1.3 eq) in DMF (2.5 mL) was added, and the reaction temperature was left stirring at 0° C. for 1 hour. Water (80 mL) was added to reaction mixture and extracted with EtOAc (80 mL). The organic layer was washed with H₂O (2×80 mL), and then with brine (80 mL). The organic layer was separated, dried over Na₂SO₄, filtered, and concentrated under vacuum. Crude product (944.8 mg) was purified using Biotage SP purification system (10 g column, fraction size: 6 mL, weak eluant: n-hexane, strong eluant: n-hexane:EtOAc=6:1, elution of product at 5% of strong eluant) to afford N-2-(trimethylsilyl)ethyl-pyrrole-2-carbaldehyde was obtained. ¹H NMR (DMSO-d₆) δ: 10.18 (s, 1H), 7.59 (m, 1H), 7.14 (m, 1H), 6.43 (m, 1H), 4.18-4.62 (m, 2H), 0.92-1.32 (m, 2H), 0.06 (s, 9H).

### b: Oxidation of N-2-(trimethylsilyl)ethyl-pyrrole-carbaldehyde

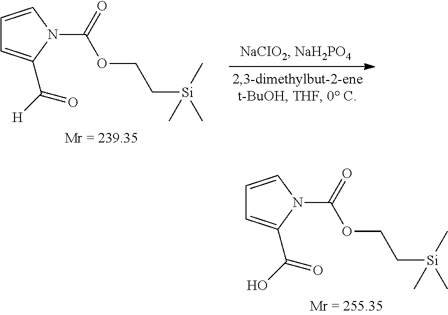 [SMILES: C[Si](C)(C)CCOC(=O)n1cccc1C=O ; C[Si](C)(C)CCOC(=O)n1cccc1C(=O)O]

To a solution of N-2-(trimethylsilyl)ethyl-pyrrole-2-carbaldehyde (420.0 mg, 1.76 mmol, 1.0 eq) in THF (15 mL) and t-BuOH (15 mL), 2,3-dimethyl-2-butene (1.67 mL, 14.1 mmol, 8.0 eq) was added at 0° C. Subsequently, a solution of sodium chlorite (478.4 mg, 5.29 mmol, 3.0 eq) and sodium dihydrogenphosphate dihydrate (825.3 mg, 5.29 mmol, 3.0 eq) in H₂O (5 mL) was added dropwise to the solution and the mixture was stirred vigorously at 0° C. for 1 hour, and then the temperature was raised to room temperature. Reaction was stirred for 5 hours at room temperature. The mixture was then diluted with a saturated solution of NH₄Cl (100 mL) and extracted with EtOAc (100 mL). Organic layer was washed with brine (100 mL), dried over Na₂SO₄, filtered, and concentrated under vacuum. Crude product (549.2 mg) was purified using Biotage SP purification system (10 g column, fraction size: 6 mL, weak eluant: DCM, strong eluant: 5% DCM/MeOH, elution of product at 0-20% of strong eluant) to afford N-2-(trimethylsilyl)ethyl-pyrrole-2-carboxylic acid. ¹H NMR (DMSO-d₆) δ: 12.75 (s, 1H), 7.38 (m, 1H), 6.83 (m, 1H), 6.14-6.39 (m, 1H), 4.21-4.53 (m, 2H), 1.26 (d, 1H), 0.96-1.15 (m, 1H), 0.04 (s, 9H).

### 1-tosyl-pyrrole-2-carboxylic acid

### a: synthesis of Methyl 1-tosyl-pyrrole-2-carboxylate

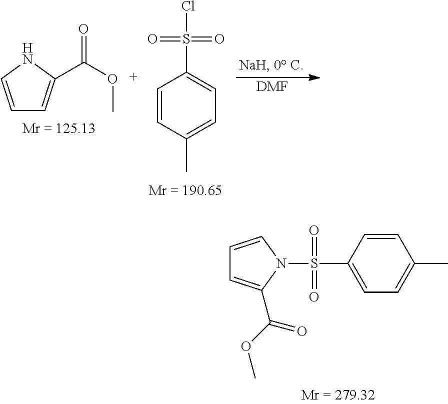

To a suspension of sodium hydride (115.2 mg, 2.88 mmol, 1.2 eq, 60% in mineral oil) in DMF (2.5 mL) methyl 2-pyrrole-carboxylate (300.0 mg, 2.40 mmol, 1.0 eq) was added at 0° C., and the mixture was stirred at the same temperature for 20 minutes. A solution of 4-toluenesulfonyl chloride (549.1 mg, 2.88 mmol, 1.2 eq) in DMF (2.5 mL) was added, and the reaction temperature was left stirring at 0° C. for 3 hours. Water (80 mL) was added to reaction mixture and extracted with EtOAc (80 mL). The organic layer was washed with brine (80 mL), dried over Na₂SO₄, filtered, and concentrated under vacuum. The crude product (757.2 mg, purity: >90%) was triturated with n-hexane (10 mL), and the resulting precipitate was separated by filtration to afford methyl 1-tosyl-pyrrole-2-carboxylate

¹H NMR (DMSO-d₆) δ: 7.94-7.99 (m, 2H), 7.93 (m, 1H), 7.55 (m, 2H), 7.18 (m, 1H), 6.50-6.60 (m, 1H), 3.75 (s, 3H), 2.49 (s, 3H).

### b: Hydrolysis of Methyl 1-tosyl-pyrrole-2-carboxylate

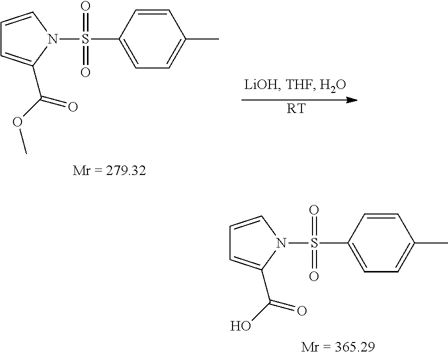 [SMILES: COC(=O)c1cccn1S(=O)(=O)c1ccc(C)cc1 ; "RT" ; Cc1ccc(S(=O)(=O)n2cccc2C(=O)O)cc1]

To a solution of methyl 1-tosyl-pyrrole-2-carboxylate (490.0 mg, 1.75 mmol, 1.0 eq) in THF (7.5 mL) a solution of LiOH (84.0 mg, 3.51 mmol, 2.0 eq) in H₂O (2.5 mL) was added at room temperature. After 40 hours of stirring, reaction mixture was poured into H₂O (100 mL), and pH was adjusted with 2M HCl to 3. Acidic aqueous layer was extracted with EtOAc (2×80 mL). The organic layers were combined, dried over Na₂SO₄, filtered, and concentrated under vacuum. The crude product (0.45 g) was triturated with n-hexane (15 mL), the resulting precipitate was separated by filtration to afford 1-tosyl-pyrrole-2-carboxylic acid.

### 1-tosyl-pyrrole-2-carboxylic acid

### a: Synthesis of 1-Tosyl-pyrrole-2-carbaldehyde

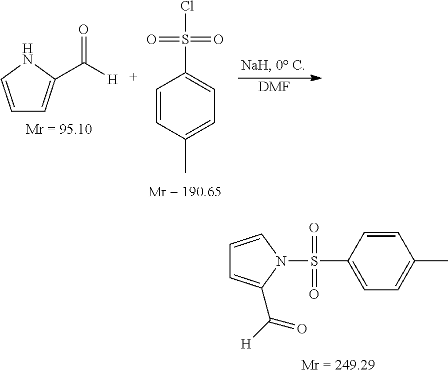

To a suspension of sodium hydride (151.6 mg, 3.15 mmol, 1.2 eq, 60% in mineral oil) in DMF (2.5 mL) 1H-pyrrole-2-carbaldehyde (300.0 mg, 3.15 mmol, 1.0 eq) was added at 0° C., and the mixture was stirred at the same temperature for 20 minutes. A solution of 4-toluenesulfonyl chloride (722.6 mg, 3.79 mmol, 1.2 eq) in DMF (2.5 mL) was added, and the reaction temperature was left stirring at 0° C. for 1 hour. Water (80 mL) was added to reaction mixture and extracted with EtOAc (80 mL). The organic layer was washed with brine (80 mL), dried over Na₂SO₄, filtered, and concentrated under vacuum. The crude product (887.0 mg, purity: 90%) was triturated with n-hexane (10 mL), the resulting precipitate was separated by filtration to afford 1-tosyl-pyrrole-2-carbaldehyde. ¹H NMR (DMSO-d₆) δ: 9.97 (s, 1H), 8.03 (d, 2H), 7.98 (m, 1H), 7.57 (m, 2H), 7.38 (m, 1H), 6.67 (m, 1H), 2.49 (s, 3H).

### b: Oxidation of 1-tosyl-pyrrole-2-carbaldehyde

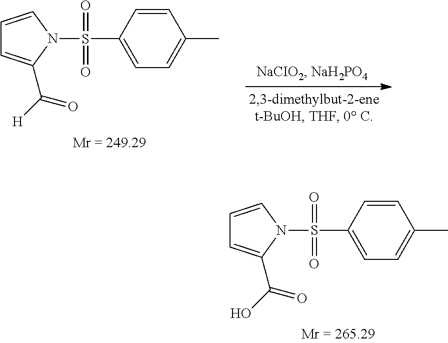 [SMILES: Cc1ccc(S(=O)(=O)n2cccc2C=O)cc1 ; Cc1ccc(S(=O)(=O)n2cccc2C(=O)O)cc1]

To a solution of 1-tosyl-pyrrole-2-carbaldehyde (0.61 g, 2.45 mmol, 1.0 eq) in THF (25 mL) and t-BuOH (25 mL) 2,3-dimethyl-2-butene (2.33 mL, 19.6 mmol, 8.0 eq) was added at 0° C. Subsequently, a solution of sodium chlorite (0.66 g, 7.34 mmol, 3.0 eq) and sodium dihydrogenphosphate dihydrate (1.15 g, 7.34 mmol, 3.0 eq) in H₂O (8 mL) was added dropwise to the solution and the mixture was stirred vigorously at 0° C. for 1 hour, and then was left to reach RT. After 16 hours of stirring, mixture was diluted with a saturated solution of NH₄Cl (100 mL) and extracted with EtOAc (100 mL). Organic layer was washed with brine (100 mL), dried over Na₂SO₄, filtered, and concentrated under vacuum. Crude product (971.5 mg) was triturated with n-hexane (15 mL, the resulting precipitate was separated by filtration to afford 1-tosyl-pyrrole-2-carboxylic acid. ¹H NMR (DMSO-d₆) δ: 12.50-12.90 (m, 1H), 7.93 (d, 2H), 7.87 (m, 1H), 7.45-7.58 (m, 2H), 7.11 (m, 1H), 6.46-6.54 (m, 1H), 2.49 (s, 3H).

### Method B: General Method for C-8-Hydroxyl Group Acylation

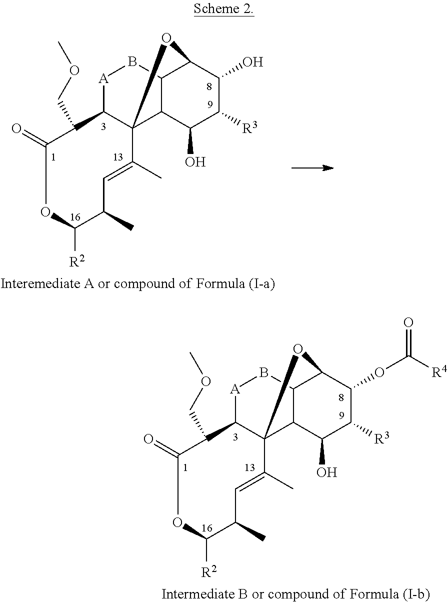

Wherein A, B and R³ are as defined for Formula (I); in Intermediate A: R² is CR^{2a}R^{2b}R^{2c}, R^{2a} is OR^{p}, R^{p} is SiEt₃, R^{2b} is H, R^{2c} is CH₂OCH₃; in compound of Formula (I-a): R² is H, or CR^{2a}R^{2b}R^{2c}, R^{2a} is H, R^{2b} is H, R^{2c} is CH₂OCH₃; in Intermediate B: R² is H or CR^{2a}R^{2b}R^{2c} is H or OR^{p}, R^{p} is SiEt₃, R^{2b} is H, R^{2c} is CH₂OCH₃, R⁴ is as defined for Formula (I), or R⁴ is R^{4p}, wherein R^{4p} is Boc-pyrrole-2-yl or (S)-4,4-difluoro-pyrrolidine-2-yl-1-carboxylic acid 1-tert-butyl ester, provided that when R² is H or R^{2a} is H, R⁴ is R^{4p}; compound of Formula (I-b): R² is H or CR^{2a}R^{2b}R^{2c} is H, R^{2b} is H, R^{2c} is CH₂OCH₃, R⁴ is as defined for Formula (I).

### Intermediate B

### Method B.a: General Method for C/8-O-Acylation Using Trichloroacetyl Reagent R⁴—C(═O)CCl₃

To a solution of starting compound (Intermediate A or compound of Formula I-a) (1.0 eq.) in DMF at 0° C. under N₂ is added NaH (4.0 eq.). The reaction mixture is stirred for 10 to 30 min before adding the appropriate trichloroacetyl reagent R⁴—C(═O)CCl₃ (1.2-1.25 eq.). The reaction mixture is stirred from 0° C. to room temperature overnight. A saturated solution of NH₄Cl is added and the mixture extracted with EtOAc. The combined organic layers are dried over Na₂SO₄, filtered and concentrated under vacuum. The desired product is obtained after purification by flash chromatography or preparative TLC to afford the desired product.

Trichloroacetyl reagent R⁴—C(═O)CCl₃ is either commercially available or can be readily prepared by methods known in the art.

### Method B.b: General Method for C/8-O-Acylation Using Carboxylic Acid R⁴—C(═O)OH or R^{4p}—C(═O)OH

A solution of starting compound (Intermediate A or compound of Formula I-a) (1.0 eq.) in DCM is treated with the appropriate DCC supported (1.5-3.0 eq.), carboxylic acid R⁴—C(═O)OH or R^{4p}—C(═O)OH (1.1-3.0 eq., for example those pyrrole intermediates prepared as described above) and DMAP (0.1-3.0 eq.), and then stirred at room temperature or at 35° C. for 16 h to 5 days. If necessary more reagents are added until the reaction reaches complete conversion. The reaction mixture is filtered and the solid washed with DCM and THF. The filtrate is concentrated under vacuum. The residue can be taken up in DCM or Et₂O and washed with saturated NaHCO₃ and saturated NH₄Cl solutions, dried over Na₂SO₄, filtered and concentrated under vacuum prior to purification; or directly purified by flash chromatography or preparative TLC to afford the desired product.

To a solution of N-Boc pyrrole carboxylic acid (970 mg, 4.6 mmol, 1.15 eq.) in dry DCM (12 mL) was added at rt Et₃N (1.68 mL, 12 mmol, 3.0 eq.) followed by 2-Methyl-6-nitrobenzoic anhydride (2.04 g, 5.8 mmol, 1.45 eq.) and DMAP (cat.), the resulting solution was stirred at rt for 60 min, then was added at rt a solution of both solids 17-TES branimycin (2.38 g, 4.0 mmol, 1.0 eq.) and DMAP (538 mg, 4.4 mmol, 1.1 eq.) in dry DCM (22 mL, via the syringe pump, the syringe was rinsed with 3 mL of dry DCM after complete addition) over 60 min, the resulting yellow solution was stirred at rt overnight. The reaction was quenched with an aqueous solution of NaHCO₃ and diluted with DCM; the aqueous layer was extracted with DCM (150 mL). The combined organic layers were washed with brine, filtered through a phase separator and concentrated. The residue was purified on SiO₂ with (EtOAc/Petroleum ether: 22/78): to give as the main fraction the 8-(Boc-pyrrole-2-yl)-17-TES-branimycin.

### Representative Intermediates B

### Intermediate B.a.1: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴ is 1H-Pyrrol-2-Yl

To a solution of Intermediate A1 (250 mg, 0.414 mmol, 1.0 eq.) in DMF (4.2 mL) at 0° C. under N₂, NaH (66 mg, 1.66 mmol, 4.0 eq.) was added. The reaction mixture was stirred for 10-30 min before adding trichloroacetyl-(1H-pyrrol-2-yl) (110 mg, 0.52 mmol, 1.25 eq.). The reaction mixture was stirred from 0° C. to room temperature overnight. A saturated solution of NH₄Cl was added and the mixture extracted with EtOAc. The combined organic layers were dried over Na₂SO₄, filtered and concentrated under vacuum. Purification of the residue by flash chromatography using heptane/EtOAc (75/25 to 60/40) afforded the desired product.

### Intermediate B.b.1: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₃ and R⁴ is 1H-Pyrrol-2-Yl

This intermediate was prepared by Method B.b starting from Intermediate A2 and 1H-pyrrole-2-carboxylic acid.

### Intermediate B.b.2: Wherein A and B Together Form Bivalent Radical —CH═CH—, R² is CR^{2a}R^{2b}R^{2c}, is OR^{p}, R^{p} is SiEt₃, R^{2b} is H, R^{2c} is CH₂OCH₃, R³ is CH₂OCH₃ and R⁴ is Furan-3-Yl

This intermediate was prepared by Method B.b starting from Intermediate A1 and furan-3-carboxylic acid.

### Intermediate B.b.3: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴ is 5-Methyl-1H-Pyrrol-2-Yl

This intermediate was prepared by Method B.b starting from Intermediate A1 and 5-Methyl-1H-pyrrole-2-carboxylic acid.

### 5-Methyl-1H-pyrrole-2-carboxylic acid preparation

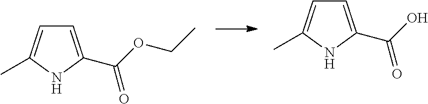

A solution of 5-methyl-1H-pyrrole-2-carboxylic acid ethyl ester (200 mg, 1.3 mmol, 1.0 eq.) in THF (2 mL) was added to a solution of LiOH (432 mg, 18 mmol, 6.0 eq.) in water (9 mL) at 55° C. The reaction mixture was stirred at 60° C. for 4 h and THF evaporated under vacuum. The reaction mixture was acidified with 3N HCl solution and extracted with EtOAc. The combined organic extracts were dried over Na₂SO₄, filtered and concentrated under vacuum to give the desired product, which was used for Intermediate B4 preparation without further purification.

### Intermediate B.b.4: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₃ and R⁴ is Furan-3-Yl

This intermediate was prepared by Method B.b starting from Intermediate A2 and 5 furan-3-carboxylic acid.

### Intermediate B.b.5: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴ is Oxazole-4-Yl

This intermediate was prepared by Method B.b starting from Intermediate A1 and oxazole-4-carboxylic acid.

### Intermediate B.b.6: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴ is Thiophene-3-Yl

This intermediate was prepared by Method B.b starting from Intermediate A1 and thiophene-3-carboxylic acid.

### Intermediate B.b.7: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴ is 1H-Pyrazole-3-Yl

This intermediate was prepared by Method B.b starting from Intermediate A1 and 1H-pyrazole-3-carboxylic acid.

### Intermediate B.b.8: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴ is 2-Methyl Furan-3-Yl

This intermediate was prepared by Method B.b starting from Intermediate A1 and 2-methyl-furan-3-carboxylic acid.

### Intermediate B.b.9: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴ is 4-Methyl-1H-Pyrrole-2-Yl

This intermediate was prepared by Method B.b starting from Intermediate A1 and 4-methyl-1H-pyrrole-2-carboxylic acid.

### Intermediate B.b.10: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴ is Isoxazole-3-Yl

This intermediate was prepared by Method B.b starting from Intermediate A1 and isoxazole-3-carboxylic acid.

### Intermediate B.b.11: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴ is Oxazole-5-Yl

This intermediate was prepared by Method B.b starting from Intermediate A1 and oxazole-5-carboxylic acid.

### Intermediate B.b.12: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴ is 5-Bromo-4-Chloro-1H-Pyrrole-2-Yl

This intermediate was prepared by Method B.b starting from Intermediate A1 and 5-Bromo-4-Chloro-1H-pyrrole-2-carboxylic acid.

### Intermediate B.b.13: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴ is 4, 5-Dibromo-1H-Pyrrole-2-Yl

This intermediate was prepared by Method B.b starting from Intermediate A1 and 4,5-Dibromo-1H-pyrrole-2-carboxylic acid.

### Intermediate B.b.14: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴ is 4-Bromo-5-Chloro-1H-Pyrrole-2-Yl

This intermediate was prepared by Method B.b starting from Intermediate A1 and 4-Bromo-5-Chloro-1H-pyrrole-2-carboxylic acid.

### Intermediate B.b.15: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴ is 4,5-Dibromo-Furan-2-Yl

This intermediate was prepared by Method B.b starting from Intermediate A1 and 4,5-Dibromo-furane-2-carboxylic acid.

### Intermediate B.b.16: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴ is 5-Methyl-1H-Pyrazole-3-Yl

This intermediate was prepared by Method B.b starting from Intermediate A1 and 5-Methyl-1H-Pyrazole-3-carboxylic acid.

### Intermediate B.a.2: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴ is 4-Cyano-1H-Pyrrole-2-Yl

This intermediate was prepared by Method B.a starting from Intermediate A1 and trichloroacetyl-(4-cyano-1H-pyrrol-2-yl).

### Trichloroacetyl-(4-cyano-1H-pyrrol-2-yl) preparation

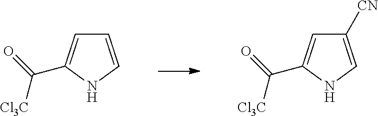

To a solution of trichloroacetyl-(1H-pyrrol-2yl) (1.5 g, 7.0 mmol, 1.0 eq.) in MeCN (15 mL) at 0° C. under N₂, chlorosulfonyl isocyanate (1.32 mL, 15 mmol, 2.15 eq.) was added. The reaction mixture was warmed up to room temperature and stirred for 3 h. DMF (5 mL) was added and resulting solution stirred overnight. Then, water was added and the reaction mixture extracted with DCM. The combined organic phases were washed with 5% NaHCO₃ aqueous solution, dried over Na₂SO₄, filtered and concentrated under vacuum. The residue was purified by flash chromatography using DCM to afford the desired product.

### Intermediate B.a.3: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2P} is CH₂OCH₃, R³ is CH₂OCH₃ and R⁴ is 4-Chloro-1H-Pyrrole-2-Yl

This intermediate was prepared by Method B.a starting from Intermediate A1 and trichloroacetyl-(4-chloro-1H-pyrrol-2-yl).

### Preparation of trichloroacetyl-(4-chloro-1H-pyrrol-2-yl) and trichloroacetyl-(4,5-di-chloro-1H-pyrrol-2-yl)

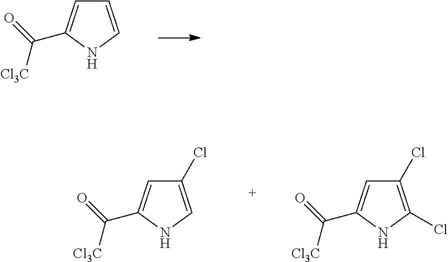

To a solution of trichloroacetyl-(1H-pyrrol-2yl) (300 mg, 1.41 mmol, 1.0 eq.) in chloroform (2 mL) sulfuryl chloride (150 μL, 1.84 mmol, 1.3 eq.) was added. The reaction mixture was allowed to stir at room temperature overnight. Then, water was added and the mixture extracted with DCM. The combined organic phases were washed with brine, dried over Na₂SO₄, filtered and concentrated under vacuum. The residue was purified by flash chromatography using heptane/EtOAc (99/1 to 95/5) to afford 4-chloro-1H-pyrrole-2-carboxylic acid and 4,5-di-chloro-1H-pyrrole-2-carboxylic acid as separate products.

### Intermediate B.a.4: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₃ and R⁴ is 4-Chloro-1H-Pyrrole-2-Yl

This intermediate was prepared by Method B.a starting from Intermediate A2 and trichloroacetyl-(4-chloro-1H-pyrrol-2-yl).

### Intermediate B.a.5: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴ is 4,5-Di-Chloro-1H-Pyrrole-2-Yl

This intermediate was prepared by Method B.a starting from Intermediate A1 and trichloroacetyl-(4,5-di-chloro-1H-pyrrol-2-yl) (prepared as described above, Intermediate B.a.3).

### Intermediate B.a.6: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴ is 4-Bromo-1H-Pyrrole-2-Yl

This intermediate was prepared by Method B.a starting from Intermediate A1 and trichloroacetyl-(4-bromo-1H-pyrrol-2-yl).

### Intermediate B.a.7: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴ is 4-Bromo-5-Fluoro-1H-Pyrrole-2-Yl

This intermediate was prepared by Method B.a starting from Intermediate A1 and trichloroacetyl-(4-bromo-5-fluoro-1H-pyrrol-2-yl) (obtained as per Organic Letters (2012) 14, 2, p. 468)

### Intermediate B.c.1: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴═R^{4p}=Boc-Pyrrol-2-Yl

This intermediate was prepared by Method B.b starting from Intermediate A1 and pyrrole-1,2-dicarboxylic acid 1-tert-butyl ester (obtained as per Tetrahedron Letters (1987) 28, 48, p. 6025).

### Intermediate B.c.2: Wherein A and B Together Form Bivalent Radical —CH₂CH₂—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴═R^{4p}=Boc-Pyrrol-2-Yl

This intermediate was prepared by Method B.b starting from Intermediate A3 and pyrrole-1,2-dicarboxylic acid 1-tert-butyl ester (obtained as per Tetrahedron Letters (1987) 28, 48, p. 6025).

### Intermediate B.c.3: Wherein A and B Together Form Bivalent Radical —CH═CH—, R² is H, R³ is CH₂OCH₃ and R⁴═R^{4p}═ is Boc-Pyrrol-2-Yl

This intermediate was prepared by Method B.b starting from Compound 21 and pyrrole-1,2-dicarboxylic acid 1-tert-butyl ester (obtained as per Tetrahedron Letters (1987) 28, 48, p. 6025).

### Intermediate B.d.1: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₂OCH₃ and R⁴═R^{4p}═(S)-4,4-Difluoro-Pyrrolidine-2-Yl-1-Carboxylic Acid 1-Tert-Butyl Ester)

This intermediate was prepared by Method B.b starting from Intermediate A1 and (S)-4,4-Difluoro-pyrrolidine-1,2-dicarboxylic acid 1-tert-butyl ester.

A solution of Intermediate A1 (615 mg, 1.03 mmol, 1.0 eq.) in DCM (20 mL) was treated with supported DCC (1.0 g, 1.56 mmol, 1.5 eq.), (S)-4,4-difluoro-pyrrolidine-1,2-dicarboxylic acid 1-tert-butyl ester (388 mg, 1.54 mmol, 1.5 eq.) and DMAP (190 mg, 1.55 mmol, 1.5 eq.) and stirred at room temperature overnight. Then, additional amount of reagents was added (0.4 eq. each) and the reaction stirred until complete conversion. The reaction mixture was filtered and the solid washed with DCM and THF. The filtrate was concentrated under vacuum and the residue purified by flash chromatography (using pentane/EtOAc 8:2 to 7:3) to afford the desired product.

### Preparation of (S)-4,4-difluoro-pyrrolidine-1,2-dicarboxylic acid 1-tert-butyl ester

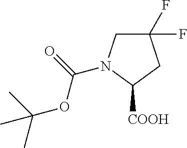 [SMILES: CC(C)(C)OC(=O)N1CC(F)(F)C[C@H]1C(=O)O]

N-Boc-4,4-Difluoro-L-proline methyl ester (1.0 g, 3.77 mmol, 1.0 eq.) was dissolved in THF/MeOH (2 mL/2 mL) and treated with a solution of lithium hydroxide monohydrate (316 mg, 7.53 mmol, 2.0 eq.) in water (4 mL). The resulting mixture was stirred at room temperature for 1 h, cooled to 0° C., acidified with 1N HCl solution, and then extracted three times with EtOAc. The combined organic phases were dried over Na₂SO₄, filtered and concentrated under vacuum to give the desired product, which was used in the next step without further purification.

### Intermediate B.d.2: Wherein A and B Together Form Bivalent Radical —CH═CH—, R² is CR^{2a}R^{2b}R^{2c}, R^{2a} is OR^{p}, R^{p} is SiEt₃, R^{2b} is H, R^{2c} is CH₂OCH₃, R³ is CH₃ and R⁴═R^{4p}═(S)-4,4-Difluoro-Pyrrolidine-2-Yl-1-Carboxylic Acid 1-Tert-Butyl Ester)

This intermediate was prepared by Method B.b starting from Intermediate A2 and (S)-4,4-Difluoro-pyrrolidine-1,2-dicarboxylic acid 1-tert-butyl ester (prepared as described above).

### Intermediate B.e.1: 8-N-2-(trimethylsilyl)ethyl-pyrrole-17-TES-branimycin

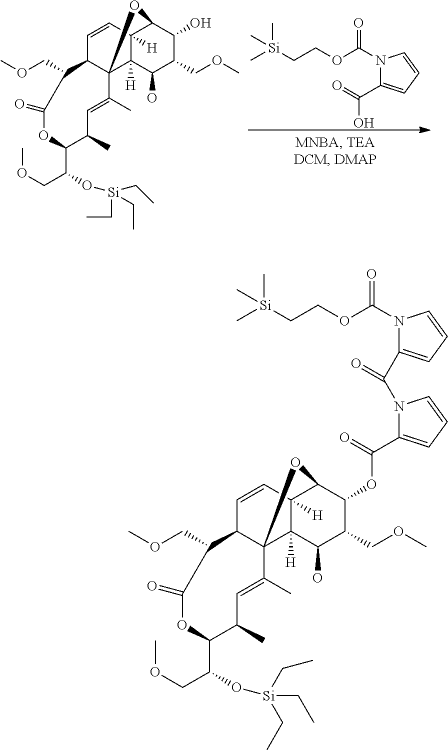

N-2-(Trimethylsilyl)ethyl-pyrrole-2-carboxylic acid (70.0 mg, 0.21 mmol, 1.3 eq), 2-methyl-6-nitrobenzoic anhydride (116.4 mg, 0.34 mmol, 1.6 eq) and triethylamine (88.5 μL, 0.63 mmol, 3.0 eq) were stirred at RT in DCM (5.0 mL) for 2 hours. Then a solution of 17-TES-branimycin (126 mg, 0.21 mmol, 1.0 eq) and DMAP (28.4 mg, 0.23 mmol, 1.1 eq) in DCM (2.5 mL) were added dropwise over 2.5 hours, and reaction mixture was left stirring at rt. After 16 hours of stirring, reaction mixture was poured into a saturated solution of NaHCO₃ (50 mL), and extracted with DCM (2×50 mL). The organic layers were combined, washed with brine (75 mL) dried over Na₂SO₄, filtered, and concentrated under vacuum. The crude product was purified on Biotage SP purification System (10 g column, fraction size: 6 mL, weak eluant: n-hexane, strong eluant: n-hexane:EtOAc=4:1, elution of product at 50% of strong eluant) to afford 8-N-2-(trimethylsilyl)ethyl-pyrrole-17-TES-branimycin.

### Intermediate B.e.2: 8-N-Ts-pyrrole-17-TES-branimycin

### Esterification of 17-TES Branimycin with N-tosyl-pyrrole-2-carboxylic acid

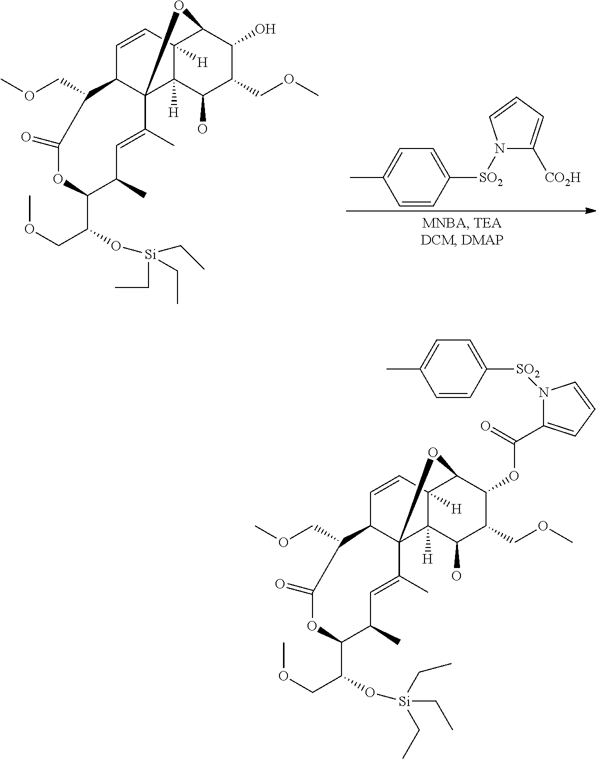

N-Ts-pyrrole-2-carboxylic acid (350.2 mg, 1.32 mmol, 1.3 eq), 2-methyl-6-nitrobenzoic anhydride (557.7 mg, 1.62 mmol, 1.6 eq) and triethylamine (426.5 μL, 3.05 mmol, 3.0 eq) were stirred at room temperature in DCM (22.5 mL) for 2 hours. Then a solution of 17-TES-Branimycin (606.0 mg, 1.02 mmol, 1.0 eq) and DMAP (136.8 mg, 1.12 mmol, 1.1 eq) in DCM (7.5 mL) were added dropwise over 3 hours, and reaction mixture was left stirring at room temperature. After 2 hours of stirring, reaction mixture was poured into a saturated solution of NaHCO₃ (70 mL), and extracted with DCM (2×50 mL). Organic layers were combined, washed with brine (80 mL) dried over Na₂SO₄, filtered, and concentrated under vacuum. The crude product was purified on Biotage SP purification system (25 g column, fraction size: 9 mL, weak eluant: n-hexane, strong eluant: n-hexane:EtOAc=2:1, elution of product at 75% of strong eluant) to afford 8-N-Ts-pyrrole-17-TES-branimycin.

### Method F: General Method for Selective C-17 Protecting Group Removal

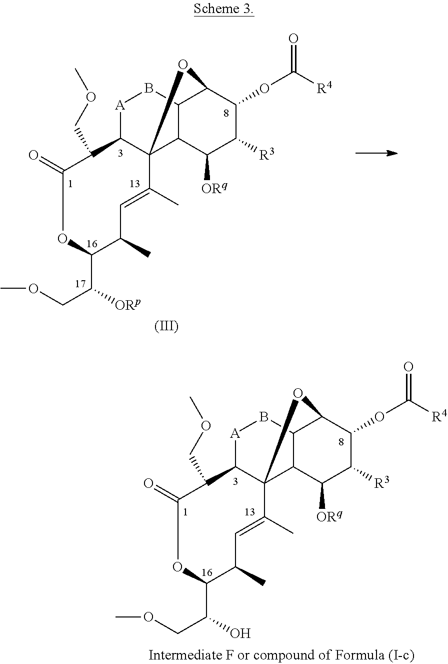

wherein A, B and R³ are as defined for Formula (I), R⁴ is as defined for Formula (I) or R⁴ is R^{4p}, wherein R^{4p} is Boc-pyrrole-2-yl or (S)-4,4-difluoro-pyrrolidine-2-yl-1-carboxylic acid 1-tert-butyl ester, and R^{q} is H or CH₂OCH₃; in Intermediate F: R⁴ is Boc-pyrrole-2-yl or (S)-4,4-difluoro-pyrrolidine-2-yl-1-carboxylic acid 1-tert-butyl ester, and R^{q} is H or CH₂OCH₃; and in Formula (I-c): R⁴ is as defined for Formula (I) and R^{q} is H

A solution of starting compound (1.0 eq.) in THF at 0° C. or room temperature is treated with a 1M solution of TBAF in THF (1.2-3.2 eq.), and allowed to stir at 0° C. or at room temperature for 10 min to 4 h. The reaction mixture is then concentrated to dryness, or diluted with DCM, washed with brine or with saturated solution of NH₄Cl, dried over Na₂SO₄, filtered and concentrated under vacuum. The residue is purified by flash chromatography to afford the desired product.

### Intermediate F1: Wherein A and B Together Form Bivalent Radical —CH═CH—, R³ is CH₂OCH₃, R⁴═R^{4p}=Boc-Pyrrol-2-Yl and R⁴ is H

This intermediate was prepared by Method F starting from Intermediate B.c.1.

### Intermediate F2: Wherein A and B Together Form Bivalent Radical —CH═CH—, R³ is CH₂OCH₃, R⁴═R^{4p}=Boc-Pyrrol-2-Yl and R^{q} is CH₂OCH₃

This intermediate was prepared by Method F starting from Intermediate M1.

### Method G: General Method for Simultaneous Conversion of —C/8-OC(═O)R^{4p} to —C/8-OC(═O)R⁴, and, if Present, of —C/17-O—R^{p} to —C/17-OH (Removal of Protective Groups)

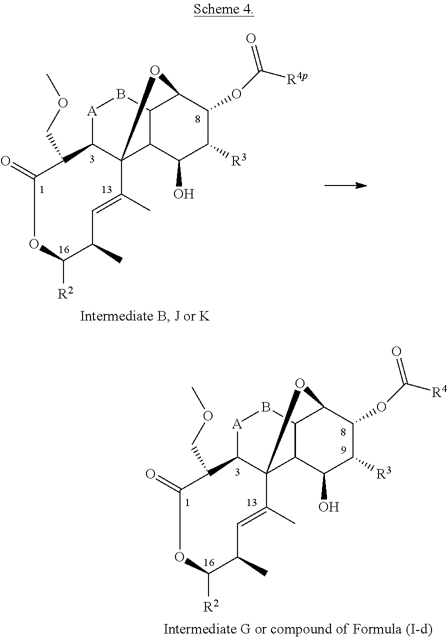

R^{4p} is

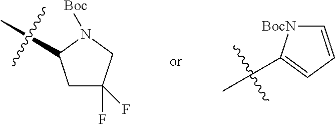

### Intermediate G: R⁴ is

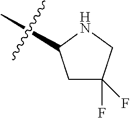

compound of Formula (I-d): R⁴ is

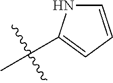

wherein A, B, and R³ are as described for Formula (I); and in Intermediate B: R² is H or CR^{2a}R^{2b}R^{2c}, R^{2a} is H or OR^{p}, R^{p} is SiEt₃, R^{2b} is H, R^{2c} is CH₂OCH₃, and R⁴ is R^{4p}, wherein R^{4p} is Boc-pyrrole-2-yl or (S)-4,4-difluoro-pyrrolidine-2-yl-1-carboxylic acid 1-tert-butyl ester, provided that when R² is H or R^{2a} is H, R⁴ is R^{4p}; in Intermediate J: R² is CR^{2a}R^{2b}R^{2c}, R^{2a} is OCH₃, R^{2b} is H, R^{2c} is CH₂OCH₃, R⁴ is R^{4p}; in Intermediate K: R² is CR^{2a}R^{2b}R^{2c}, R^{2a} and R^{2b} together form oxo, R^{2c} is CH₂OCH₃, R⁴ is R^{4p}; in Intermediate G: R² is H or CR^{2a}R^{2b}R^{2c}, R^{2a} is H, OH or OCH₃, R^{2b} is H, or R^{2a} and R^{2b} together form oxo, R^{2c} is CH₂OCH₃, R⁴ is (S)-4,4-difluoro-pyrrolidine-2-yl; in compound of Formula (I-d): R² is H or CR^{2a}R^{2b}R^{2c}, R^{2a} is H, OH or OCH₃, R^{2b} is H, or R^{2a} and R^{2b} together form oxo, R^{2c} is CH₂OCH₃, R⁴ is 1H-pyrrole-2-yl.

A solution of starting compound (1.0 eq.) in DCM at 0° C. is treated with TFA (50 eq.) and stirred at 0° C. for 30 min to 1 h. The reaction mixture is concentrated under vacuum and the residue is purified by flash chromatography or preparative TLC to afford the desired product.

### Intermediate G1: Wherein A and B Together Form Bivalent Radical —CH═CH—, R² is CR^{2a}R^{2b}R^{2c}, R^{2a} is OH, R^{2b} is H, R^{2c} is CH₂OCH₃, R³ is CH₂OCH₃, R⁴ is (S)-4,4-Difluoro-Pyrrolidine-2-Yl

This intermediate was prepared by Method G starting from Intermediate B.d.1.

A solution of Intermediate B.d.1. (400 mg, 0.48 mmol, 1.0 eq.) in DCM (40 mL) at 0° C. was treated with TFA (50 eq.) and stirred at 0° C. for 30 min. The reaction mixture was concentrated under vacuum and the residue purified by flash chromatography (using DCM/MeOH 9:1) to afford the desired product.

### Intermediate G2: Wherein A and B Together Form Bivalent Radical —CH═CH—, R² is CR^{2a}R^{2b}R^{2c}, R^{2a} is OH, R^{2b} is H, R^{2c} is CH₂OCH₃, R³ is CH₃, R⁴ is (S)-4,4-Difluoro-Pyrrolidine-2-Yl

This intermediate was prepared by Method G starting from Intermediate B.d.2.

### Method G3. Protecting Group Removal

### Method G3.a: Protecting Group Removal

To a cooled solution of the 8-(Boc-pyrrole-2-carbonyl)-17-TES-branimycin (2.2 g, 2.79 mmol) in dry DCM (50 mL) at 0° C. was added TFA (11 mL, 144 mmol, 50 eq.) over 15 min and the resulting solution was stirred at 0° C. for 75 min. The reaction was quenched at 0° C. by slow addition of K₂CO₃ solid (˜12 g) until complete neutralization, the mixture was stirred at 0° C. for 5 min and at rt for 20 min, diluted with DCM and filtrated. The K₂CO₃ solid was washed with DCM until no product could be detected and the filtrate was concentrated. The residue was purified on SiO₂ with (Et₂O/Petroleum ether/Acetone: 80/10/10): to give the desired compound.

A solution of starting compound (1.0 eq.) in THF at 0° C. or room temperature is treated with a 1M solution of TBAF in THF (1.2-3.2 eq.), and allowed to stir at 0° C. or at room temperature for 10 min to 4 h. The reaction mixture is then concentrated to dryness, or diluted with DCM, washed with brine or with a saturated solution of NH₄Cl, dried over Na₂SO₄, filtered and concentrated under vacuum. The residue is purified by flash chromatography to afford the desired product.

### Method G4: General Method for Simultaneous Deprotection of —C/8 and, if Present, of —C/17 (Removal of Protective Groups)

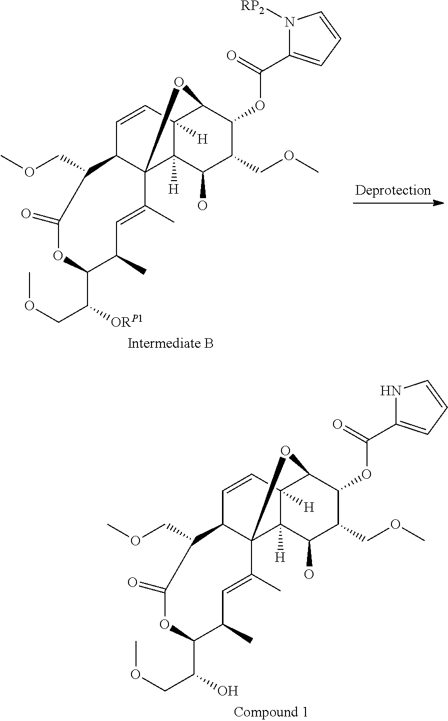

A solution of starting compound (1.0 eq.) in DCM at 0° C. is treated with TFA (50 eq.) and stirred at 0° C. for 30 min to 1 h. The reaction mixture is concentrated under vacuum and the residue is purified by flash chromatography or preparative TLC to afford the desired product.

### Method G4.a: Deprotection of 8-N-2-(trimethylsilyl)ethyl-pyrrole-17-TES-Branimycin with TFA

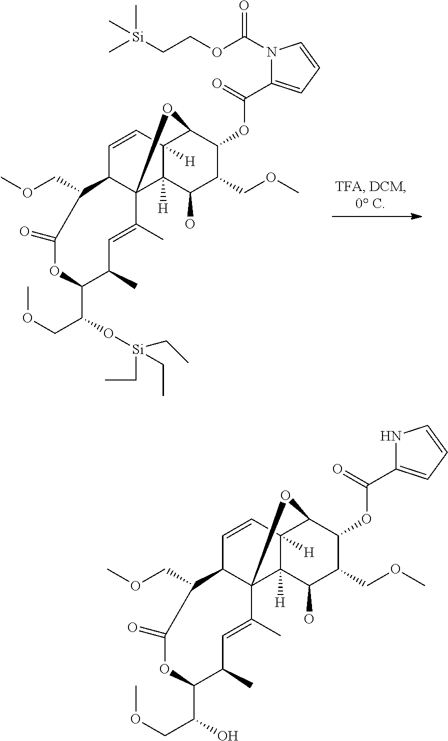 [SMILES: CC[Si](CC)(CC)O[C@H](COC)[C@H]1OC(=O)[C@H](COC)[C@H]2C=C[C@H]3[C@H]4O[C@]2(/C(C)=C/[C@H]1C)[C@@H]3[C@H](O)[C@@H](COC)[C@H]4OC(=O)c1cccn1C(=O)OCC[Si](C)(C)C ; COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1ccc[nH]1]

8-N-2-(Trimethylsilyl)ethyl-pyrrole-17-TES-branimycin (150.0 mg, 0.18 mmol, 1.0 eq) was dissolved in DCM (5 mL) at 0° C., and trifluoroacetic acid (668.0 μL, 9.0 mmol, 50.0 eq) was added. After 30 minutes, reaction was stopped by adding K₂CO₃ (s, 746.3 mg, 5.4 mmol, 30.0 eq) to the reaction mixture at 0° C. Reaction was left stirring at 0° C. for 5 minutes, and then 10 minutes at room temperature. Reaction mixture was diluted with DCM (25 mL), and filtered. Solid was washed with DCM, until no product could be detected (checked by UV). The filtrate was concentrated under vacuum to give a white foam (111.2 mg), which was purified on a Biotage SP purification system (10 g column, fraction size: 6 mL, strong eluant: 5% MeOH/DCM, weak eluant: DCM, elution of product at 50% of strong eluant) to afford desired product.

### Method G4.b: Deprotection of 8-N-2-(trimethylsilyl)ethyl-pyrrole-17-TES-Branimycin with TASF

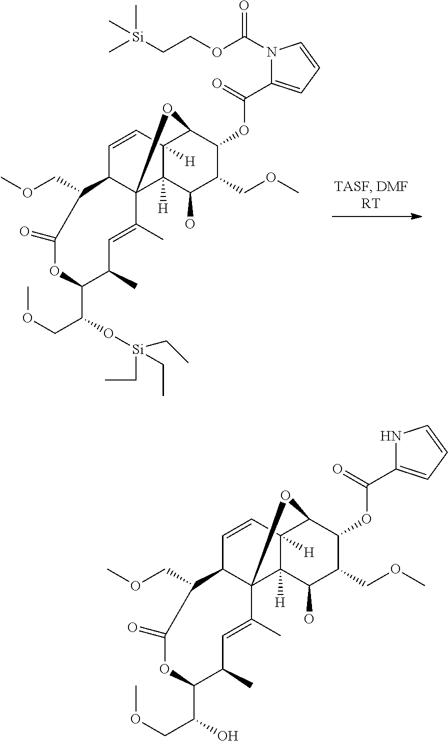 [SMILES: CC[Si](CC)(CC)O[C@H](COC)[C@H]1OC(=O)[C@H](COC)[C@H]2C=C[C@H]3[C@H]4O[C@]2(/C(C)=C/[C@H]1C)[C@@H]3[C@H](O)[C@@H](COC)[C@H]4OC(=O)c1cccn1C(=O)OCC[Si](C)(C)C ; COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1ccc[nH]1]

8-N-2-(Trimethylsilyl)ethyl-pyrrole-17-TES-branimycin (1.60 mg, 1.32 μmol, 1.0 eq) was dissolved in DMF (0.2 mL) and tris(dimethylamino)sulfonium difluorotrimethylsilicate (2.64 mg, 6.59 μmol, 5.0 eq) was added. Reaction mixture was monitored by OAUPLC-MS and TLC (eluant: 5% MeOH/DCM). After 2 hours of stirring at room temperature, the reaction proceeded quantitatively.

### Method G4.c: Deprotection of N-Tosyl-Pyrrole-17-TES-Branimycin with TASF

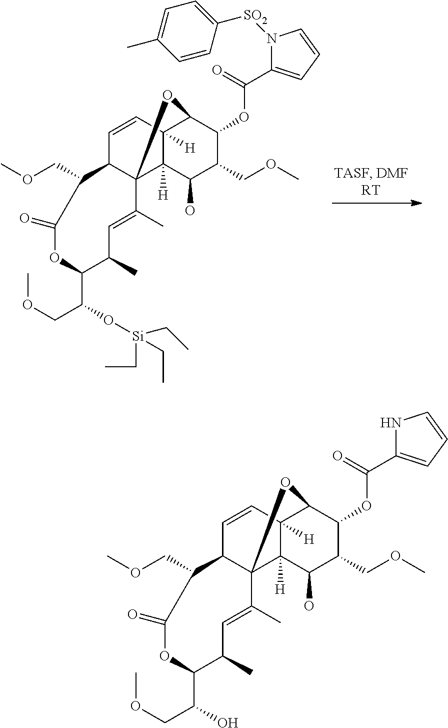 [SMILES: CC[Si](CC)(CC)O[C@H](COC)[C@H]1OC(=O)[C@H](COC)[C@H]2C=C[C@H]3[C@H]4O[C@]2(/C(C)=C/[C@H]1C)[C@@H]3[C@H](O)[C@@H](COC)[C@H]4OC(=O)c1cccn1S(=O)(=O)c1ccc(C)cc1 ; COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1ccc[nH]1]

8-N-Ts-pyrrole-17-TES-branimycin (1.0 mg, 1.18 μmol, 1.0 eq) was dissolved in DMF (0.2 mL) and tris(dimethylamino)sulfonium difluorotrimethylsilicate (1.63 mg, 5.92 μmol, 5.0 eq) was added. Reaction mixture was monitored by UPLC-MS and TLC (eluant: 5% MeOH/DCM). After 7 hours of stirring at room temperature, the deprotection was completed.

### Method H: General Method for Aromatization of (S)-4,4-Difluoro-Pyrrolidine-2-Yl

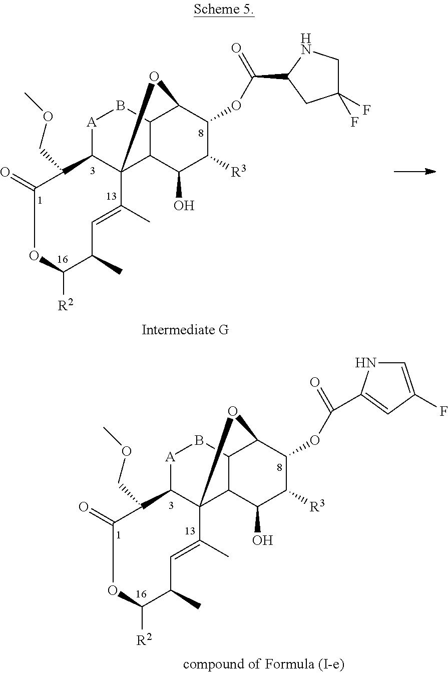

wherein A, B, and R³ are as described for Formula (I), R² is H or CR^{2a}R^{2b}R^{2c}, wherein R^{2a} is H, OH, or OCH₃, R^{2b} is H, or R^{2a} and R^{2b} together form oxo, and R^{2c} is CH₂OCH₃.

Activated MnO₂ (8-10 eq.) is added to a solution of Intermediate G (1.0 eq.) in THF at room temperature. The resulting suspension is refluxed for 1.5-3 h. The reaction mixture is filtered through celite, rinsed with THF and concentrated under vacuum. The residue is purified by flash chromatography or preparative TLC to afford the desired product.

### Method I: General Method for C-4/C-5-Hydrogenation

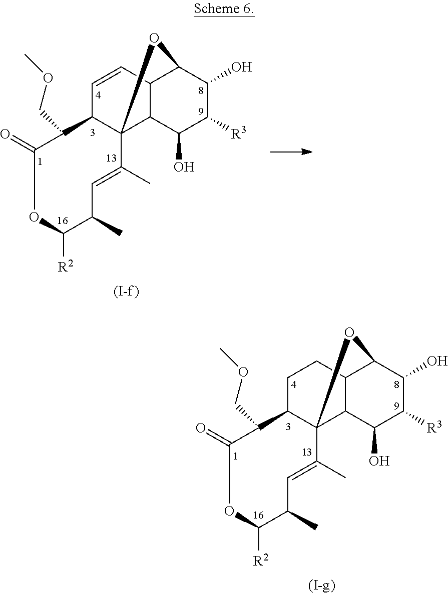

wherein R² is H or CR^{2a}R^{2b}R^{2c}, wherein R^{2a} is H or OH, R^{2b} is H, R^{2c} is CH₂—O—CH₃, and R³ is as described for Formula (I).

### General Method I for C-4/C-5-Hydrogenation

A solution of starting compound (I-f) (1.0 eq.) in EtOAc is reduced by hydrogenation (full H₂, room temperature, 1 mL/min) using a 10% Pd/C cartridge. The solvent is removed under vacuum to afford the desired product.

### Method J: General Method for C-17-Hydroxyl Group Methylation

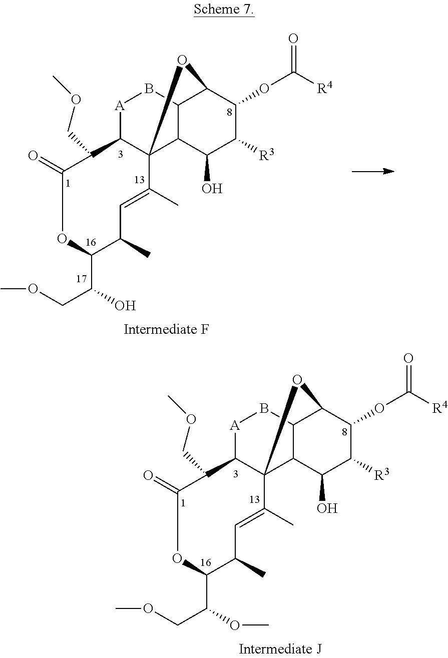

wherein A, B, R³ and R⁴ are as described for Formula (I), or R⁴ is R^{4p}, wherein R^{4p} is Boc-pyrrole-2-yl.

A solution of Intermediate F (1.0 eq.) in DCE is treated at room temperature with 4 \acute{Å} molecular sieves, bis-1,8-dimethylaminophtalene (2.5 eq.), then trimethyloxonium tetrafluoroborate (2.5-3 eq.) and stirred at room temperature for 15-24 h. The reaction mixture is filtered through Celite and the solvent is removed under reduced pressure. The residue is purified by flash chromatography or preparative TLC to afford the desired mono C-17-OMe product.

### Intermediate J1: Wherein A and B Together Form Bivalent Radical —C═C—, R³ is CH₂OCH₃, R⁴ is R^{4p}=Boc-Pyrrol-2-Yl

This intermediate was prepared by Method J starting from Intermediate F1.

A solution of Intermediate F1 (100 mg, 0.148 mmol, 1.0 eq.) in DCE (2.2 mL) was treated at room temperature with 4\acute{Å} molecular sieves (100 mg), bis-1,8-dimethylaminophtalene (79 mg, 0.370 mmol, 2.5 eq.), then trimethyloxonium tetrafluoroborate (58 mg, 0.395 mmol, 2.67 eq.) and stirred at room temperature for 22 h. The reaction mixture was filtered through Celite and the solvent removed under reduced pressure. The residue was purified by two preparative TLC (using successively pentane/EtOAc 1:1 and CHCl₃/MeOH 95:5) to give the desired product.

### Method K: General Method for C-17-Hydroxyl Group Oxidation

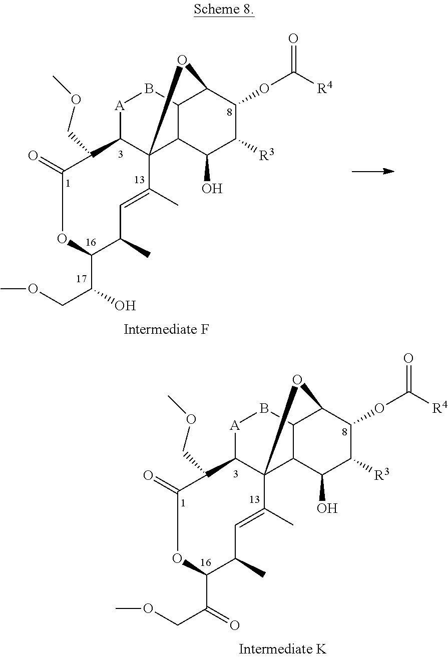

wherein A, B, R³ and R⁴ are as described for Formula (I), or R⁴ is R^{4p}, wherein R^{4p} is Boc-pyrrole-2-yl.

A solution of Intermediate F (1.0 eq.) in DCM is treated with TEMPO (0.1-0.3 eq.) and BAIB (1.1-2.5 eq.). The reaction mixture is stirred from room temperature to 40° C. for 48 h-7 days. The mixture is diluted in DCM and a saturated solution of Na₂S₂O₃ added. The layers are separated and the aqueous phase is extracted three times with DCM. The combined organic layers are washed with brine, dried over Na₂SO₄, filtered and concentrated under vacuum. The residue is purified by flash chromatography or preparative TLC to afford the desired product.

### Intermediate K1: Wherein A and B Together Form Bivalent Radical —C═C—, R³ is CH₂OCH₃, R⁴ is R^{4p}=Boc-Pyrrol-2-Yl

This intermediate was prepared by Method K starting from Intermediate F1.

A solution of Intermediate F1 (135 mg, 0.20 mmol, 1.0 eq.) in DCM (1.2 mL) was treated with TEMPO (3 mg, 0.020 mmol, 0.1 eq.) and BAIB (71 mg, 0.22 mmol, 1.1 eq.). The reaction mixture was stirred at room temperature for 48 h and at 40° C. for 18 h. More TEMPO (5 mg, 0.033 mmol, 0.17 eq.) was added and the reaction mixture stirred at 40° C. for 72 h. More BAIB (71 mg, 0.22 mmol, 1.1 eq.) was added and the reaction mixture stirred at 40° C. for 18 h. The mixture was diluted in DCM and a saturated solution of Na₂S₂O₃ added. The layers were separated and the aqueous phase was extracted three times with DCM. The combined organic layers were washed with brine, dried over Na₂SO₄, filtered and concentrated under vacuum. The residue was purified by preparative TLC using DCM/EtOAc 55:45 to give the desired product.

### Method L: General Method for C-17/C-18 Region Cleavage

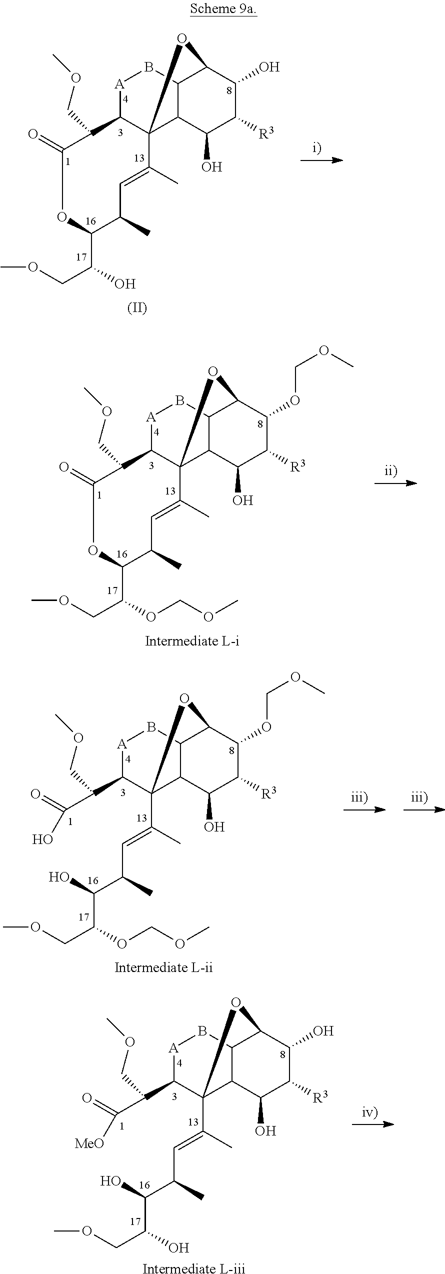

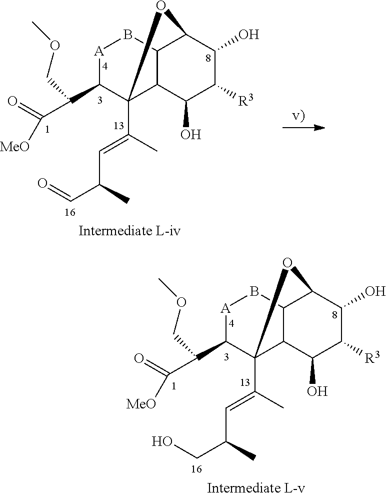

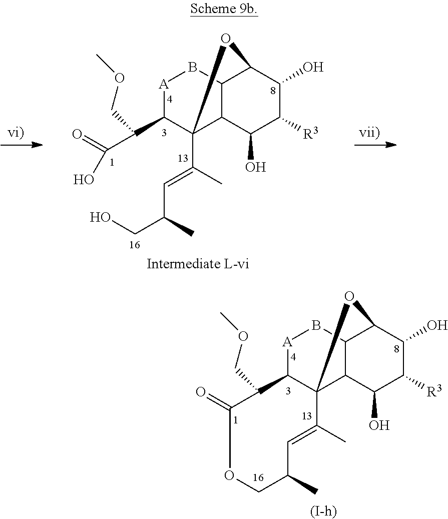

wherein A, B, and R³ are as described for Formula (I).

### Intermediate L-i

A solution of starting compound (1.0 eq.) in DCM at 0° C. is treated with DIPEA (14.5 eq.) and chloromethyl methylether (9.9 eq.) and stirred from 0° C. to room temperature for 72 h. A saturated solution of NaHCO₃ is added and the mixture extracted three times with DCM. The combined organic layers are dried over Na₂SO₄, filtered and concentrated under vacuum. The residue is purified by flash chromatography or preparative TLC to afford the desired product.

### Intermediate L-ii

A solution of Intermediate L-i, step i (1.85 g, 3.15 mmol, 1.0 eq.) in acetone/MeOH/H₂O (2/2/1) at room temperature is treated with KOH (155.6 eq.) and stirred at 60° C. overnight. Water is added and the mixture extracted with DCM. The aqueous layer is then acidified with concentrated HCl and extracted with DCM. The combined organic layers are dried over Na₂SO₄, filtered and concentrated under vacuum. The crude product is used directly in the next step without further purification.

### Intermediate L-iii

A solution of Intermediate L-ii, step ii (1.0 eq.) in MeOH at room temperature is treated with a 37% HCl solution and heated at 60° C. overnight. The reaction mixture is concentrated under vacuum and diluted with DCM and water. The aqueous layer is separated and extracted with DCM. The combined organic layers are dried over Na₂SO₄, filtered and concentrated under vacuum. The residue is purified by flash chromatography or preparative TLC to afford the desired product.

### Intermediate L-iv

A solution of Intermediate L-iii, step iii (1.0 eq.) in acetone/water (1/1) is treated at room temperature with sodium periodate (2.8 eq.) and the reaction mixture stirred at room temperature overnight. Acetone is evaporated under reduced pressure and the aqueous layer extracted with DCM. The combined organic layers are dried over Na₂SO₄, filtered and concentrated under vacuum to give the desired product, which was used in the next step without further purification.

### Intermediate L-v

A solution of Intermediate L-iv, step iv (1.0 eq.) in THF at 0° C. is treated with sodium borohydride (3.5 eq.) and the reaction mixture is stirred from 0° C. to room temperature overnight. A saturated solution of NH₄Cl is added and the mixture extracted with DCM. The combined organic layers are dried over Na₂SO₄, filtered and concentrated under vacuum to give the desired product, which was used in the next step without further purification.

### Intermediate L-vi

A solution of Intermediate L-v, step v (1.0 eq.) in THF/MeOH/H₂O (2/2/1) at room temperature is treated with a 2M aqueous NaOH solution (8.0 eq.) and stirred at 70° C. overnight. The reaction mixture is concentrated under vacuum and diluted with water. The solution is washed twice with DCM. The aqueous layer is acidified with a 2N solution of HCl and extracted with DCM and EtOAc. The combined organic layers are dried over Na₂SO₄, filtered and concentrated under vacuum to give the desired product, which was used in the next step without further purification.

### Compound of Formula (I), Wherein R² is H

Intermediate L-vi, step vi (1.0 eq.) is dissolved in dry toluene/THF (5/1) and 2,2′-dithiodipyridine (5.0 eq.) and triphenylphosphine (5.0 eq.) are added. The reaction mixture is stirred at room temperature for 16 h. The reaction mixture is diluted with toluene (14 mL) and added over 3 h with a syringe pump to a suspension of silver perchlorate (10.0 eq.) in degassed toluene at 80° C. The reaction mixture is then filtered through Celite, washed with toluene and concentrated under vacuum. The residue is purified by successive flash chromatography to give the desired product, which can be purified by methods know to the skill in the art, or can be used as such in the next step.

### Method M: General Method for C-10-Hydroxyl Group Protection

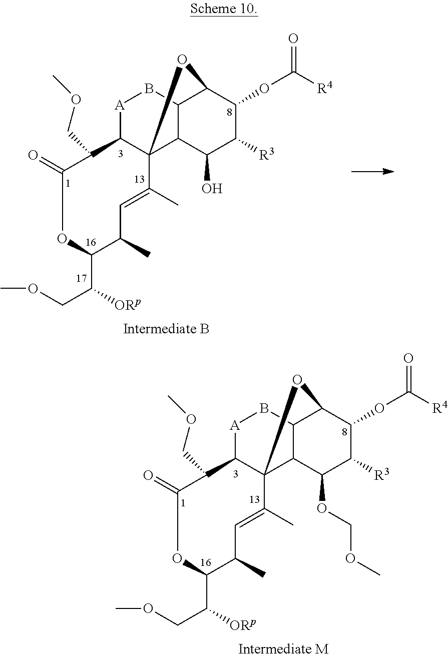

wherein R^{p} is SiEt₃, and A, B, R³ and R⁴ are as described for Formula (I), or R⁴ is R^{4p}, wherein R^{4p} is Boc-pyrrole-2-yl.

A solution of Intermediate B (1.0 eq.) in DCM at 0° C. is treated with DIPEA (15-45 eq.) and chloromethyl methylether (10-30 eq.) and stirred from 0° C. to room temperature for 48 h to 7 days. A saturated solution of NaHCO₃ is added and the mixture extracted three times with DCM. The combined organic layers are washed with a saturated solution of NH₄Cl, dried over Na₂SO₄, filtered and concentrated under vacuum. The residue is purified by flash chromatography or preparative TLC to afford the desired product.

### Intermediate M1: Wherein A and B Together Form Bivalent Radical —CH═CH—, R^{p} is SiEt₃, R³ is CH₂OCH₃ and R⁴ is R^{4p}=Boc-Pyrrol-2-yl

A solution of Intermediate B.c.1 (1.0 g, 1.266 mmol, 1.0 eq.) in DCM (55 mL) at 0° C. was treated with DIPEA (3.14 mL, 18.99 mmol, 15.0 eq.) and chloromethyl methylether (0.96 mL, 12.66 mmol, 10.0 eq.) and stirred from 0° C. to room temperature for 48 h. DIPEA and chloromethyl methylether (resp. 15.0 eq. and 10.0 eq.) were added and the reaction mixture was stirred at room temperature for 72 h. DIPEA and chloromethyl methylether (resp. 15.0 eq. and 10.0 eq.) were added again and stirring of reaction mixture continued at room temperature for 48 h. A saturated solution of NaHCO₃ was added and the mixture extracted three times with DCM. The combined organic layers were washed with a saturated solution of NH₄Cl, dried over Na₂SO₄, filtered and concentrated under vacuum. The residue was purified by flash chromatography (using pentane/EtOAc 1:0 to 1:1) to give the desired product.

### Method N: Alternative General Method for C-17-Hydroxyl Group Oxidation

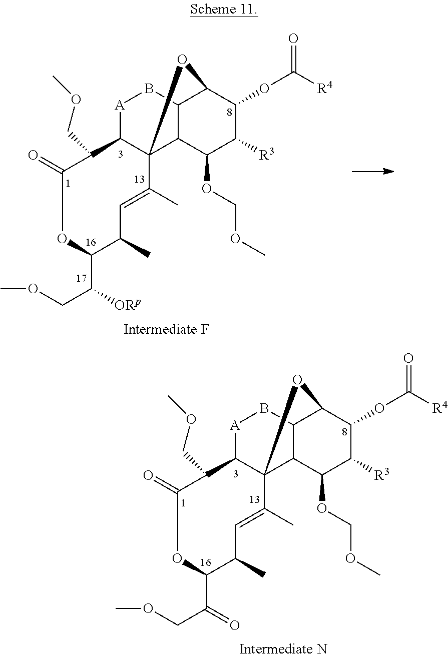

wherein R^{p} is SiEt₃, and A, B, R³ and R⁴ are as described for Formula (I), or R⁴ is R^{4p}, wherein R^{4p} is Boc-pyrrole-2-yl

A solution of Intermediate F (1.0 eq.) in DCM at 0° C. is treated with Dess-Martin periodinane (2.0 eq.) and pyridine (5.0 eq.). The mixture is stirred from 0° C. to room temperature overnight. To the reaction mixture are added successively Et₂O, a saturated aqueous solution of Na₂S₂O₃ and a saturated aqueous solution of NaHCO₃. The mixture is stirred at room temperature until it becomes clear. The layers are separated and the aqueous phase is extracted with Et₂O. The combined organic layers are washed with NaHCO₃ and brine, dried over Na₂SO₄, filtered and concentrated under vacuum to afford the desired product.

### Intermediate N1: Wherein A and B Together Form Bivalent Radical —CH═CH—, R³ is CH₂OCH₃ and R⁴═R^{4p} is Boc-Pyrrol-2-Yl

A solution of Intermediate F2 (160 mg, 0.222 mmol, 1.0 eq.) in DCM (5 mL) at 0° C. was treated with Dess-Martin periodinane (188 mg, 0.444 mmol, 2.0 eq.) and pyridine (90 μL, 1.111 mmol, 5.0 eq.). The mixture was stirred from 0° C. to room temperature overnight. To the reaction mixture were added successively Et₂O, a saturated aqueous solution of Na₂S₂O₃ and a saturated aqueous solution of NaHCO₃. The mixture was stirred at room temperature until it becomes clear. The layers were separated and the aqueous phase was extracted with Et₂O. The combined organic layers were washed with NaHCO₃ and brine, dried over Na₂SO₄, filtered and concentrated under vacuum (evaporation with toluene) to afford the desired product.

### Method P: General Method for C-17-Methylation

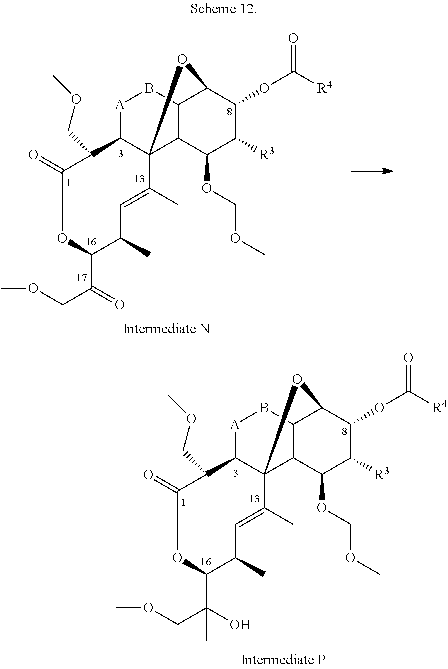

wherein A, B, and R³ are as described for Formula (I), in Intermediate N: R⁴ is as described for Formula (I) or R⁴ is R^{4p}, which is Boc-pyrrole-2-yl; in Intermediate P: R⁴ is as described for Formula (I).

### Intermediate P

A solution of Intermediate N (1.0 eq.) in dry THF at −78° C. is treated with methyl magnesium bromide (3.0 M in diethyl ether, 1.2 eq.) and allowed to stir from −78° C. to room temperature overnight. A saturated solution of NH₄Cl is added and the mixture extracted twice with EtOAc. The combined organic layers are washed with brine, dried over Na₂SO₄, filtered and concentrated under vacuum to give crude product which may be, if desired, further purified by standard techniques.

### Intermediate P1: Wherein A and B Together Form Bivalent Radical —CH═CH—, R³ is CH₂OCH₃ and R⁴ is Pyrrol-2-Yl

A solution of Intermediate N1 (94 mg, 0.131 mmol, 1.0 eq.) in dry THF (2 mL) at −78° C. was treated with methyl magnesium bromide (3.0 M in diethyl ether, 52 μL, 0.157 mmol, 1.2 eq.) and allowed to stir from −78° C. to room temperature overnight. A saturated solution of NH₄Cl was added and the mixture extracted twice with EtOAc. The combined organic layers were washed with brine, dried over Na₂SO₄, filtered and concentrated under vacuum. The residue is purified by preparative TLC (using pentane/EtOAc 3:7) to give the desired product.

### Method O: General Method for Oximation of C-17-Oxo

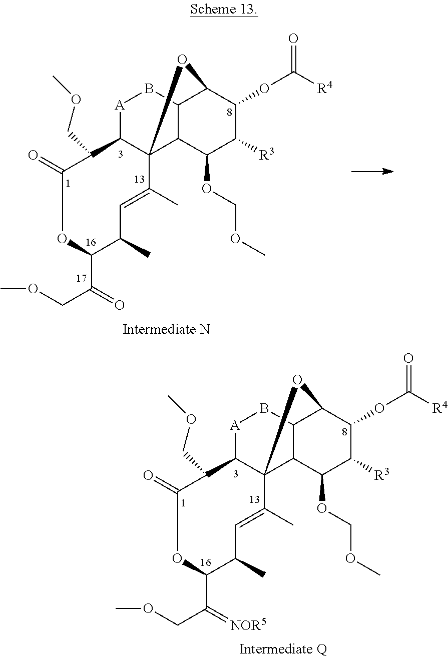

wherein A, B, R³ and R⁴ are as described for Formula (I), or R⁴ is R^{4p}, which is Boc-pyrrole-2-yl.

To a solution of Intermediate N (1.0 eq.) in pyridine the appropriate hydroxylamine hydrochloride (3.0 eq.) is added. The reaction mixture is stirred at room temperature for 3 h. A saturated solution of NH₄Cl is added and the mixture extracted with DCM. The combined organic layers are washed with brine, dried over Na₂SO₄, filtered and concentrated under vacuum to give the desired product as a mixture of Z and E isomers.

### Intermediate Q1: Wherein A and B Together Form Bivalent Radical —CH═CH—, R³ is CH₂OCH₃ and R⁴ is R^{4p}=Boc-Pyrrol-2-Yl, and R⁵ is H

To a solution of Intermediate N1 (170 mg, 0.22 mmol, 1.0 eq.) in pyridine (3 mL) NH₂OH×HCl (46 mg, 0.66 mmol, 3.0 eq.) was added. The reaction mixture is stirred at room temperature for 3 h. A saturated solution of NH₄Cl was added and the mixture extracted with DCM. The combined organic layers were washed with brine, dried over Na₂SO₄, filtered and concentrated under vacuum (co-evaporation with toluene) to give the desired product as a mixture of Z and E isomers.

### Intermediate Q2: Wherein A and B Together Form Bivalent Radical —CH═CH—, R³ is CH₂OCH₃ and R⁴ is R^{4p}=Boc-Pyrrol-2-Yl, and R⁵ is CH₃

This intermediate was prepared by Method Q starting from Intermediate N1 and CH₃ONH₂×HCl.

### Method Y: General Method for C-17-Hydroxyl Group Protection by Thiocarbonyl Imidazole

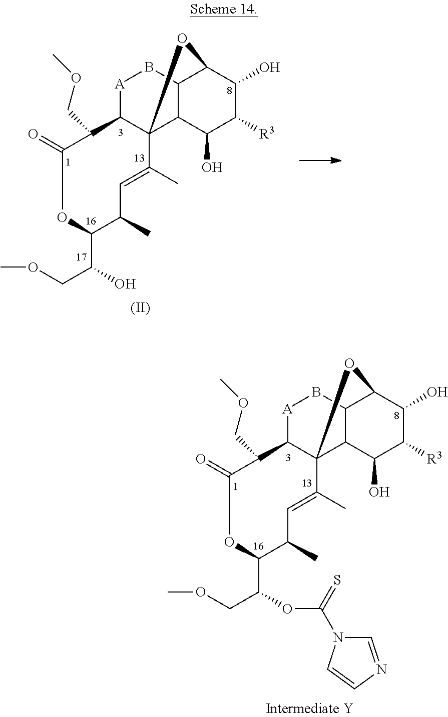

wherein A, B, and R³ are as described for Formula (I).

A solution of starting compound (1.0 eq.) in THF (5 mL) at room temperature is treated with pyridine (2.6-6 eq.), DMAP (0.1-0.2 eq.) and TCDI (1.3-1.6 eq.) and stirred at 63° C. for 48 h-5 days. The reaction mixture is cooled to room temperature, diluted with DCM and washed successively with a saturated solution of NaHCO₃ and with a 0.5N HCl solution. The organic layer is dried over Na₂SO₄, filtered and concentrated under vacuum to give crude product which may be, if desired, further purified by standard techniques.

### Intermediate Y1: Wherein A and B Together Form Bivalent Radical —C═C—, R³ is CH₂OCH₃

A solution of Branimycin (101 mg, 0.209 mmol, 1.0 eq.) in THF (5 mL) at room temperature was treated with pyridine (44 μL, 0.54 mmol, 2.6 eq.), DMAP (3 mg, 0.02 mmol, 0.1 eq.) and TCDI (49 mg, 0.27 mmol, 1.3 eq.) and stirred at 63° C. for 48 h. The reaction mixture was cooled to room temperature, diluted with DCM and washed with a saturated solution of NaHCO₃. The organic layer was dried over Na₂SO₄, filtered and concentrated under vacuum. The TLC (solvents: DCM:MeOH=95:5) of the obtained residue revealed that starting material was still present. Therefore same amount of each reagent was added and stirring continued for additional 72 h at 63° C. The reaction mixture was cooled to room temperature, diluted with DCM and first washed with saturated NaHCO₃ solution and then with 0.5N HCl solution. The organic layer was dried over Na₂SO₄, filtered and concentrated under vacuum. The residue was purified by preparative TLC (using EtOAc) to give the desired product.

### Method W: General Method for C-17-Hydroxyl Group Removal

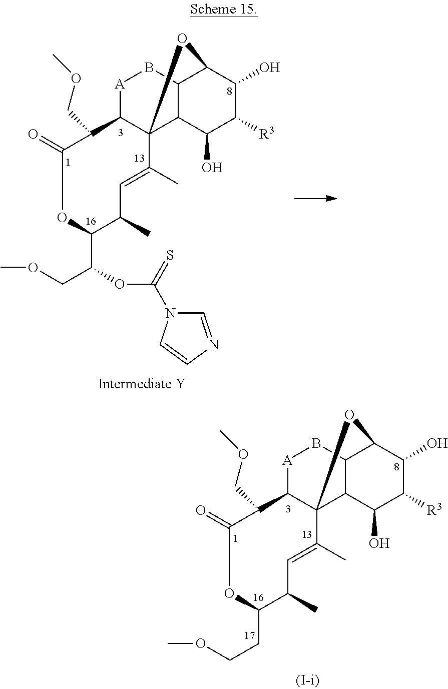

wherein A, B, and R³ are as described for Formula (I).

A solution of Intermediate Y (1.0 eq.) in THF is added portion wise (6 portions over 30 min) to a solution of tributyltin hydride (6.0 eq.) in THF at 60° C. The reaction mixture is stirred at 60° C. for 30 min and concentrated to 0.5 mL under vacuum to give crude product which may be further purified by standard techniques.

### Method Z: General Method of Simultaneous Removal of C-10-Hydroxyl Protective Group and, if Present Conversion of —C/8-O—C(═O)R^{4p} to —C/8-O—C(═O)R⁴

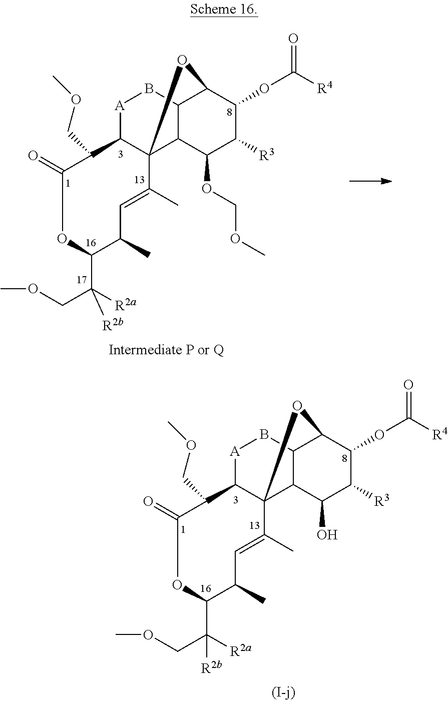

wherein A, B, and R³ are as described for Formula (I); in Intermediate P: R^{2a} is OH, R^{2b} is CH₃, R⁴ is as described for Formula (I); in Intermediate Q: R^{2a} and R^{2b} together form ═N—OH or ═N—OCH₃, R⁴ is as described for Formula (I) or R⁴ is R^{4p}, which is Boc-pyrrole-2-yl; compound of formula (I-j): R^{2a} is OH, R^{2b} is CH₃, or R^{2a} and R^{2b} together form ═N—OH or ═N—OCH₃, R⁴ is as described for Formula (I).

A solution of starting compound (1.0 eq.) in DCM was treated at 0° C. to room temperature with a 4N solution of HCl in dioxane (50 eq.), and stirring continued at room temperature for 3-4 h. A saturated solution of NaHCO₃ is added and the mixture extracted twice with DCM. The combined organic layers are washed with brine, dried over Na₂SO₄, filtered and concentrated under vacuum to give crude product which may be further purified by standard techniques.

### Method R: General Method for C-17-Oxime Group Acetylation

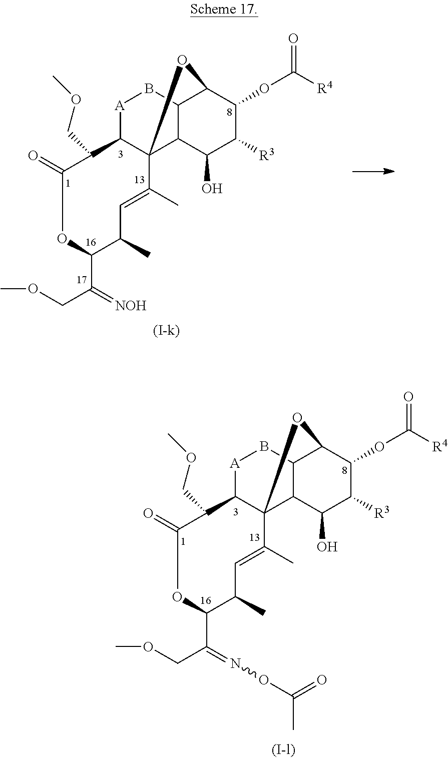

wherein A, B, R³ and R⁴ are as described for Formula (I).

To a solution of starting compound of Formula (I) where R^{2a} and R^{2b} together form ═N—OH (1.0 eq.) in AcOH (250 μL) and Ac₂O (170 μL) zinc dust (3.0 eq.) is added. The reaction mixture is stirred at room temperature overnight, then diluted with chloroform and washed with water and brine. The organic layer is dried over Na₂SO₄, filtered and concentrated under vacuum to give crude product which may be further purified by standard techniques.

### Synthesis of Representative Compounds of the Invention and Reference Compounds

### Branimycin

This compound was prepared by the following fermentation process.

For the preparation of the first seed stage, one mycelial cell bank working copy was quickly thawed at 37° C. in a water bath and the glycerol-culture of Saccharothrix xinjiangensis G60/1571 30×B2M21, registered under accession number NCIMB 41952 (12.5 mL) transferred to 1 L baffled Erlenmeyer flasks containing YM7.2 medium (200 mL). It should be recognized that other media may be used to optimize various aspects of production. This first seed stage was cultivated at 28° C. for 48 h on a rotary shaker, analyzed microscopically and pooled prior to transfer to the second seed stage. To prepare the second seed stage, a fermenter containing 20 L YM7.2 medium (200 mL) was inoculated from the first seed stage using 1% first seed stage inoculum. The second seed stage was grown for 48 h at 28° C. with an air flow rate of 14 L per min and an initial stirrer speed of 400 rpm and pooled prior to inoculation of MC production medium (3000 L).

Fermentation was carried out in a 4000 L stirred tank fermenter at 28° C. with an air flow rate of 300 L per min and an initial stirrer speed of 400 rpm. Dissolved oxygen was controlled online during fermentation. During fermentation, foaming was controlled by automatic addition of antifoaming agent Silfoam Se2, Struktol J647 and water (1:1:1). It would be appreciated by a person of skill in the art that other types of antifoaming agents can be used, such as other type of polypropylene glycols, silicones, esters, fatty acids, fats, and sulfonates. Compound production was monitored on a daily basis by use of LC-MS/UV/ELSD analysis. Fermentations were carried-out for 90 to 144 h, typically for 120 h. In the fermentations described above, any stirring system known to a person of skill in the art may be used, for example a conventional paddle stirrer, a turbine-stirrer system or the Ekato InterMIG impeller.

After harvest, around 2% Celite 512 were added to the fermentation broth. The fermentation broth was filtered through 24 chambers (17.28 m²) with a filtration performance of 526.24 L per hour. 125 L resin (LEWATIT VP OC 1064 MD PH) were loaded to a 800 L column (cross section 770 mm; height 27 cm). The supernatant was pumped through the column with a flow rate of 3500 L per hour for three hours. Afterwards the culture broth was extracted with 350 L per hour. The loaded resin was eluted two times with double volume acetone in portions. The acetone was evaporated until a residual water phase was visible. The water phase was extracted three times with equal amounts of ethyl acetate. The ethyl acetate extracts were combined and the solvent was removed under reduced pressure yielding around 750 g crude extract.

The crude extract was then subject to normal phase MPLC using a Biotage Isolera Flash purification system with silica cartridge (800 g) as the solid phase and gradient solvent mixture DCM:MeOH from 1:0 to 96:4 as eluent to obtain a branimycin-enriched fraction that was further purified on reversed phase MPLC using a Biotage Isolera Flash purification system with C18 cartridge (800 g), gradient ACN:H₂O 0% to 100% to give branimycin as a substantially pure product.

### YM7.2 Medium

| Ingredients | g/L |
|---|---|
| Glucose | 4.0 |
| Yeast extract | 4.0 |
| Malt extract | 10.0 |

Dissolve in 1000 mL of distilled H₂O

Adjust pH to 7.2 before sterilization

Autoclave

### MC Medium

| Ingredients | g/L |
|---|---|
| Glucose | 10.0 |
| Starch | 10.0 |
| Yeast extract | 5.0 |
| Soy flour defatted | 10.0 |
| NaCl | 5.0 |
| CaCO₃ | 3.0 |

Dissolve in 1000 mL of tap H₂O

Adjust pH to 7.2 before sterilization

Autoclave

### Compound 1: Baleomycin

This compound was prepared by the following fermentation process.

For the preparation of the first seed stage, one mycelial cell bank working copy was quickly thawed at 37° C. in a water bath and the glycerol-culture of Saccharothrix xinjiangensis G60/1571 30×B2M21, registered under accession number NCIMB 41952 (12.5 mL) transferred to 1 L baffled Erlenmeyer flasks containing Celmer-79a medium (200 mL). This first seed stage was cultivated at 28° C. for 48 h on a rotary shaker, analyzed microscopically and pooled prior to transfer to the second seed stage. To prepare the second seed stage, 18×1 L baffled Erlenmeyer flasks containing Celmer-79a medium (200 mL) were inoculated from the first seed stage using 5% first seed stage inoculum. The second seed stage was grown for 48 h at 28° C. on a rotary shaker (120 rpm, 10 cm stroke) and pooled prior to inoculation of SGG production medium (90 L).

Fermentation was carried out in a 140 L stirred tank fermenter at 28° C. with an air flow rate of 63 L per min and an initial stirrer speed of 200 rpm. Dissolved oxygen was cascade-controlled at 30% via agitation. Pure oxygen was added at a flow rate of 20 L per min by using the on/off control when the agitation speed reaches the maximum value. To prevent foaming during fermenter sterilization, Antifoam A (Sigma Aldrich, #10794, 30% aqueous emulsion of silicon polymer) was added to the medium at 0.1% (v/v). During fermentation, foaming was controlled by automatic addition of antifoaming agent Desmophen® 2061 BD (Bayer, solvent-free linear polypropylene ether polyol). It would be appreciated by the skill in the art that other types of antifoaming agents can be used, such as other type of polypropylene glycols, silicones, esters, fatty acids, fats, and sulfonates. Compound production was monitored on a daily basis by use of HPLC-MS/CAD analysis. Fermentations were typically carried-out for 120 h.

After harvest, the broth was then extracted 3 times for 12 h on a rotary shaker with EtOAc (1:1). Decantation of EtOAc phase and evaporation of the solvent was performed to obtain the EtOAc extract. This later extract was further solubilized with pentane (1.5 L). After decantation, the pentane phase was slowly removed to give the defatted extract as the insoluble residue.

The defatted extract was then solubilized twice with MeOH (1 L) per ˜100 g extract. After filtration, the MeOH was separated from the insoluble residue and dried to obtain the final extract.

The equivalent of 100 g of final extract was then treated with MtBE (1 L) and filtrated. The residue was then treated with DCM (500 mL) and NH₄OH (500 mL). The DCM phase was collected and dried down to provide crude extract.

The crude extract was then subject to normal phase MPLC using a Biotage Isolera Fash purification system with silica cartridge (800 g) as the solid phase and gradient solvent mixture DCM:MeOH from 1:0 to 96:4 as eluent to obtain a baleomycin-enriched fraction that was further purified on preparative HPLC using a Varian preparative HPLC system, a Nucleodur Sphinx C18 250×40 mm column, gradient ACN:H₂O 10% to 100% to give compound 1 (baleomycin) as a pure product.

Celmer-79a medium

| Ingredients | g/L |
|---|---|
| Glucose* | 10.0 |
| Starch | 20.0 |
| Yeast extract | 5.0 |
| Meat extract | 5.0 |
| Tryptone | 5.0 |
| K₂HPO₄ | 0.5 |
| CoCl₂ × 6H₂0 | 0.004 |
| CaCO₃ | 4.0 |

*added after sterilization from sterile stock solution (c = 0.2 g/mL)

Dissolve in 1000 mL of distilled H₂O

Adjust pH to 7.2 before sterilization

Autoclave

SGG medium

| Ingredients | g/L |
|---|---|
| Glucose* | 10.0 |
| Glycerol | 10.0 |
| Starch | 10.0 |
| Cornsteep powder | 2.5 |
| Yeast extract | 2.0 |
| Tryptone | 5.0 |
| NaCl | 1.0 |
| CaCO₃ | 3.0 |

*added after sterilization from sterile stock solution (c = 0.3 g/mL)

Dissolve in 1000 mL of tap H₂O

Adjust pH to 7.2 before sterilization

Sterilize

### Compound 2: 8-O-1H-Pyrrole-2′-Carbonylbranimycin

This compound was prepared by Method F starting from Intermediate B.a.1.

A solution of Intermediate B.a.1 (740 mg, 0.937 mmol, 1.0 eq.) in THF (37 mL) at 0° C. was treated with a 1M solution of TBAF in THF (1.41 mL, 1.41 mmol, 1.5 eq.). The reaction mixture was stirred at 0° C. for 2.5 h, then diluted with DCM, washed with brine, dried over Na₂SO₄, filtered and concentrated at room temperature under vacuum. The residue was purified by flash chromatography (using DCM/EtOAc 1:0 to 0:1) to afford the desired product.

### Compound 3

This compound was prepared by Method F starting from Intermediate B.b.1.

### Compound 4

This compound was prepared by Method B.b starting from Compound 17.

### Compound 5

This compound was prepared by Method F starting from Intermediate B.b.2.

### Compound 6

This compound was prepared by Method F starting from Intermediate B.b.3.

### Compound 7

This compound was prepared by Method F starting from Intermediate B.b.4.

### Compound 8

This compound was prepared by Method F starting from Intermediate B.b.5.

### Compound 9

This compound was prepared by Method F starting from Intermediate B.b.6.

### Compound 10

This compound was prepared by Method F starting from Intermediate B.b.7.

### Compound 11

This compound was prepared by Method F starting from Intermediate B.b.8.

### Compound 12

This compound was prepared by Method F starting from Intermediate B.b.9.

### Compound 13

This compound was prepared by Method F starting from Intermediate B.b.10.

### Compound 14

This compound was prepared by Method F starting from Intermediate B.b.11.

### Compound 15

This compound was prepared by Method I starting from Branimycin.

A solution of Branimycin (170 mg, 0.34 mmol, 1.0 eq.) in EtOAc (6 mL) was reduced by hydrogenation (full H₂, room temperature, 1 mL/min) using a 10% Pd/C cartridge. The solvent is removed under vacuum to afford the desired product.

### Compound 16

This compound was prepared by Method G starting from Intermediate B.c.2.

A solution of Intermediate B.c.2. (27 mg, 0.034 mmol, 1.0 eq.) in DCM (4 mL) at 0° C. was treated with TFA (0.13 mL, 1.7 mmol, 50 eq.) and stirred at 0° for 50 min. The reaction mixture was concentrated under vacuum and the residue purified by preparative TLC (using CHCl₃/MeOH 95:5) to afford the desired product.

### Compound 17

This compound was prepared by Method W starting from Intermediate Yl.

A solution of Intermediate Yl (39 mg, 0.067 mmol, 1.0 eq.) in THF (1 mL) was added portion wise (6 portions over 30 min) to a solution of tributyltin hydride (100 μL, 0.40 mmol, 6.0 eq.) in THF (2 mL) at 60° C. The reaction mixture was stirred at 60° C. for 30 min and concentrated to 0.5 mL under vacuum. The residue was purified by flash chromatography (using pentane/EtOAc 9:1 then 0:1) to afford the desired product.

### Compound 18

This compound was prepared by Method G starting from Intermediate J1.

### Compound 19

This compound was prepared by Method H starting from Intermediate G1.

Activated MnO₂ (308 mg, 3.5 mmol, 8.4 eq.) was added to a solution of Intermediate G1 (260 mg, 0.42 mmol, 1.0 eq.) in THF (7 mL) at room temperature. The resulting suspension was refluxed for 1.5 h. The reaction mixture was filtered through celite, rinsed with THF and concentrated under vacuum. The residue was purified by flash chromatography (using DCM/EtOAc 1:1) to afford the desired product.

### Compound 20

This compound was prepared by Method G starting from Intermediate K1.

### Compound 21: 17,18-Dinor-branimycin

This compound was prepared by Method L starting from Branimycin.

### Step i: Intermediate L-i.1

A solution of branimycin (2.05 g, 4.25 mmol, 1.0 eq.) in DCM (125 mL) at 0° C. was treated with DIPEA (10.2 mL, 0.62 mmol, 14.5 eq.) and chloromethyl methylether (3.2 mL, 42.1 mmol, 9.9 eq.), and stirred from 0° C. to room temperature for 72 h. A saturated solution of NaHCO₃ was added and the mixture extracted three times with DCM. The combined organic layers were dried over Na₂SO₄, filtered and concentrated under vacuum. The residue was purified by flash chromatography (using DCM/EtOAc) to give the desired product.

### Step ii: Intermediate L-ii.1

A solution of Intermediate L-i.1, from step i (1.85 g, 3.15 mmol, 1.0 eq.) in acetone/MeOH/H₂O (48 mL/48 mL/24 mL) was treated with KOH (27.5 g, 490 mmol, 155.6 eq.), then heated to 60° C. and stirred at this temperature overnight. Water was added and the mixture extracted with DCM. The aqueous layer was then acidified with concentrated HCl (50 mL) and extracted with DCM. The combined organic layers were dried over Na₂SO₄, filtered and concentrated under vacuum. The crude product was used in the next step without further purification.

### Step iii: Intermediate L-iii.1

A solution of Intermediate L-iii.1, from step ii (1.15 g, 1.9 mmol, 1.0 eq.) in MeOH (60 mL) at room temperature was treated with a 37% HCl solution (0.3 mL) and heated at 60° C. overnight. The reaction mixture was concentrated under vacuum and diluted with DCM and water. The aqueous layer was separated and extracted with DCM. The combined organic layers were dried over Na₂SO₄, filtered and concentrated under vacuum. The residue was purified by flash chromatography (using DCM/MeOH 1:0 to 9:1) to give the desired product.

### Step iv: Intermediate L-iv.1

A solution of Intermediate L-iii.1, from step iii (690 mg, 1.34 mmol, 1.0 eq.) in acetone/water (75 mL/75 mL) was treated at room temperature with sodium periodate (803 mg, 3.75 mmol, 2.8 eq.) and the reaction mixture stirred at room temperature overnight. Acetone was evaporated under reduced pressure and the aqueous layer extracted with DCM. The combined organic layers were dried over Na₂SO₄, filtered and concentrated under vacuum to give the desired product, which was used in the next step without further purification.

### Step v: Intermediate L-v.1

A solution of Intermediate L-iv.1, from step iv (580 mg, 1.32 mmol, 1.0 eq.) in THF (100 mL) at 0° C. was treated with sodium borohydride (175 mg, 4.63 mmol, 3.5 eq.) and the reaction mixture stirred from 0° C. to room temperature overnight. A saturated solution of NH₄Cl was added and the mixture extracted with DCM. The combined organic layers were dried over Na₂SO₄, filtered and concentrated under vacuum to give the desired product, which was used in the next step without further purification.

### Step vi: Intermediate L-vi.1

A solution of Intermediate L-v.1, from step v (495 mg, 1.12 mmol, 1.0 eq.) in THF/MeOH/H₂O (50 mL/50 mL/25 mL) at room temperature was treated with 2M aqueous NaOH solution (4.5 mL, 9.0 mmol, 8.0 eq.) and stirred at 70° C. overnight. The reaction mixture was concentrated under vacuum and diluted with water. The solution was washed twice with DCM. The aqueous layer was acidified with a 2N solution of HCl and extracted with DCM and EtOAc. The combined organic layers were dried over Na₂SO₄, filtered and concentrated under vacuum to give the desired product, which was used in the next step without further purification.

### Step vii: Compound 21: 17,18-Dinor-branimycin

Intermediate L-vii.1, from step vi (150 mg, 0.35 mmol, 1.0 eq.) is dissolved in dry toluene/THF (5 mL/1 mL) and 2,2′-dithiodipyridine (386 mg, 1.75 mmol, 5.0 eq.) and triphenylphosphine (459 mg, 1.75 mmol, 5.0 eq.) are added. The reaction mixture is stirred at room temperature for 16 h. The reaction mixture is diluted with toluene (14 mL) and added over 3 h with a syringe pump to a suspension of silver perchlorate (580 mg, 3.51 mmol, 10.0 eq.) in degassed toluene (330 mL) at 80° C. The reaction mixture is then filtered through Celite, washed with toluene and concentrated under vacuum. The residue is purified by successive flash chromatographies (using respectively DCM/MeOH 95:5 to 9:1 and DCM/EtOAc 4:1 to 1:1) to give the desired product, contaminated with triphenylphosphine derivatives, which may be purified if desired by standard techniques known to the skill in the art and which was used without further purification in the next step.

### Compound 22

This compound was prepared by Method G starting from Intermediate B.c.3.

### Compound 23

This compound was prepared by Method H starting from Intermediate G2.

### Compound 24

This compound was prepared by Method Z starting from Intermediate P1.

A solution of Intermediate P1 (20 mg, 0.032 mmol, 1.0 eq.) in DCM (1.5 mL) was treated at room temperature with a 4N solution of HCl in dioxane (0.40 mL, 1.60 mmol, 50 eq.) and stirred at this temperature for 4 h. A saturated solution of NaHCO₃ was added and the mixture extracted twice with DCM. The combined organic layers were washed with brine, dried over Na₂SO₄, filtered and concentrated under vacuum. The residue was purified by preparative TLC (using CHCl₃/MeOH 95:5, 2 migrations) to give the desired product.

### Compound 25

This compound was prepared by Method Z starting from Intermediate Q1.

A solution of Intermediate Q1 (160 mg, 0.218 mmol, 1.0 eq.) in DCM (10 mL) was treated at 0° C. with a 4N solution of HCl in dioxane (2.7 mL, 10.9 mmol, 50 eq.) and stirred whilst warming to room temperature for 3 h. A saturated solution of NaHCO₃ was added and the mixture extracted twice with DCM. The combined organic layers were washed with brine, dried over Na₂SO₄, filtered and concentrated under vacuum. The residue was purified by preparative TLC (using CHCl₃/EtOH 9:1) to give the desired product.

### Compound 26

This compound was prepared by Method F starting from Intermediate B.a.2.

### Compound 27

This compound was prepared by Method Z starting from Intermediate Q2.

### Compound 28

This compound was prepared by Method F starting from Intermediate B.a.3.

### Compound 29

This compound was prepared by Method R starting from Compound 25.

To a solution of Compound 25 (30 mg, 0.051 mmol, 1.0 eq.) in AcOH (250 μL) and Ac₂O (170 μL) zinc dust (10 mg, 0.150 mmol, 3.0 eq.) was added. The reaction mixture was stirred at room temperature overnight, then diluted with chloroform and washed with water and brine. The organic layer was dried over Na₂SO₄, filtered and concentrated under vacuum. The crude was purified by preparative TLC (using heptane/EtOAc 1:3) to give the desired product.

### Compound 30

This compound was prepared by Method F starting from Intermediate B.a.4.

### Compound 31

This compound was prepared by Method F starting from Intermediate B.a.5.

### Compound 32

This compound was prepared by Method F starting from Intermediate B.a.6.

### Compound 33

This compound was prepared by Method F starting from Intermediate B.b.12.

### Compound 34

This compound was prepared by Method F starting from Intermediate B.b.13.

### Compound 35

This compound was prepared by Method F starting from Intermediate B.b.14.

### Compound 36

This compound was prepared by Method F starting from Intermediate B.b.15.

### Compound 37

This compound was prepared by Method F starting from Intermediate B.a.7.

### Compound 38

This compound was prepared by Method used in Intermediate B.a.7 starting from Compound 19.

### Compound 39

This compound was prepared by Method F starting from Intermediate B.b.16.

### Compound 40

This compound was prepared by Method Z starting from Intermediate Q.

### Compound 41

This compound was prepared by Method Z starting from Intermediate Q.

### Compound 42

This compound was prepared by Method R starting from Compound 41.

The reference compounds are listed in Table 1A, exemplary compounds that have been or can be prepared according to the synthetic methods described herein are listed in Table 1B below. The NMR spectral data of some representative compounds of the invention is given in Table 2 below.

TABLE 1A
reference compounds

| Name | Structure | Source |
|---|---|---|
| Nargenicin A1 | 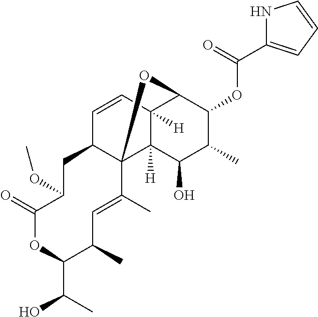 [SMILES: CO[C@H]1C[C@H]2C=C[C@H]3[C@H]4O[C@]2(/C(C)=C/[C@@H](C)[C@@H]([C@@H](C)O)OC1=O)[C@@H]3[C@H](O)[C@@H](C)[C@H]4OC(=O)c1ccc[nH]1] | Albany Molecular Research Inc (AMRI); Enzo Life Sciences Product List (Cat # ALX- 380-096) Santa Cruz Biotechnology, Inc., 10410 Finnell Street, Dallas, Texas 75220, U.S.A. (Cat n#sc-222044) |
| Nargenicin B1 | 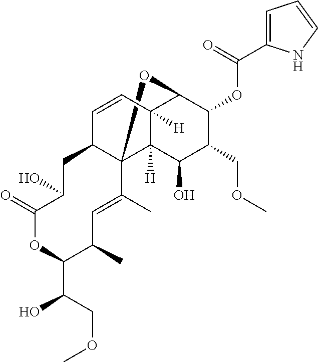 [SMILES: COC[C@@H]1[C@@H](O)[C@@H]2[C@H]3C=C[C@@H]4C[C@H](O)C(=O)O[C@H]([C@H](O)COC)[C@H](C)/C=C(\C)[C@@]24O[C@H]3[C@@H]1OC(=O)c1ccc[nH]1] | U.S. Pat. No. 4,436,747 and references therein |
| Nodusmicin | 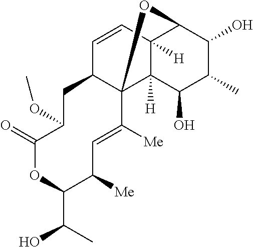 [SMILES: CO[C@H]1C[C@H]2C=C[C@H]3[C@H]4O[C@]2(/C(C)=C/[C@@H](C)[C@@H]([C@@H](C)O)OC1=O)[C@@H]3[C@H](O)[C@@H](C)[C@H]4O] | Albany Molecular Research Inc (AMRI) Or BioAustralis Fine Chemicals (BIA- N1326) |
| Branimycin | 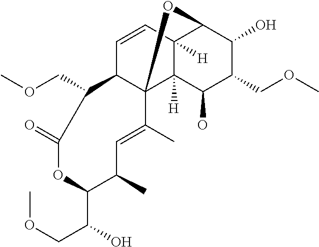 | See example above |

TABLE 1B
Compounds of the Invention

| Cpd | | | | |
|---|---|---|---|---|
| # | Structures | Name | MW | MS Mes'd |
| 1 | 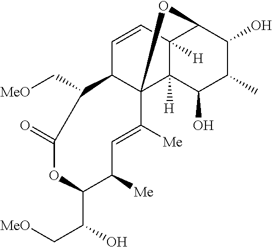 [SMILES: COC[C@@H](O)[C@H]1OC(=O)[C@H](COC)[C@H]2C=C[C@H]3[C@H]4O[C@]2(/C(C)=C/[C@H]1C)[C@@H]3[C@H](O)[C@@H](C)[C@H]4O] | Baleomycin | 452.6 | 435.2(−H₂O) |
| 2 | 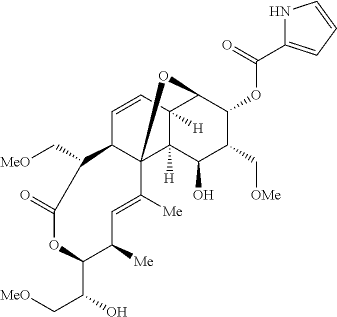 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1ccc[nH]1] | 8-O-1H-pyrrole-2′- carbonylbranimycin | 575.6 | 558.5 (−H₂O) |
| 3 | 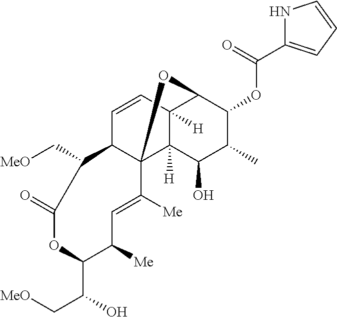 [SMILES: COC[C@@H](O)[C@H]1OC(=O)[C@H](COC)[C@H]2C=C[C@H]3[C@H]4O[C@]2(/C(C)=C/[C@H]1C)[C@@H]3[C@H](O)[C@@H](C)[C@H]4OC(=O)c1ccc[nH]1] | 8-O-1H-pyrrole-2′- carbonylbaleomycin | 545.6 | 528.4 (−H₂O) |
| 4 | 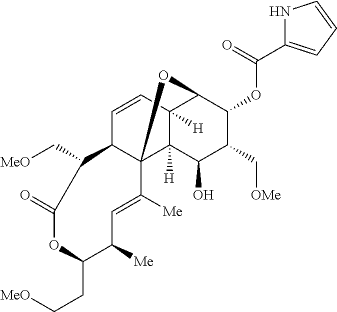 [SMILES: COCC[C@H]1OC(=O)[C@H](COC)[C@H]2C=C[C@H]3[C@H]4O[C@]2(/C(C)=C/[C@H]1C)[C@@H]3[C@H](O)[C@@H](COC)[C@H]4OC(=O)c1ccc[nH]1] | 17-Deoxy-8-O-1H- pyrrole-2′- carbonylbranimycin | 559.6 | 542.4 (−H₂O) |
| 5 | 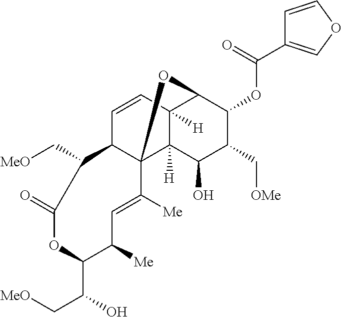 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1ccoc1] | 8-O-Furane-3′- carbonylbranimycin | 576.6 | 559.4 (−H₂O) |
| 6 | 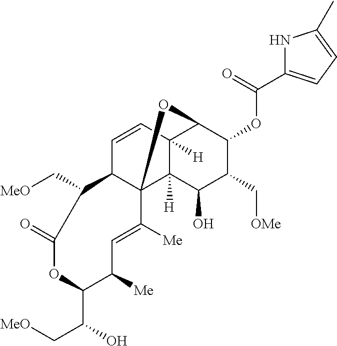 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1ccc(C)[nH]1] | 8-O-5-Methyl-1H- pyrrole-2′- carbonylbranimycin | 589.7 | 612.2 (+Na⁺) |
| 7 | 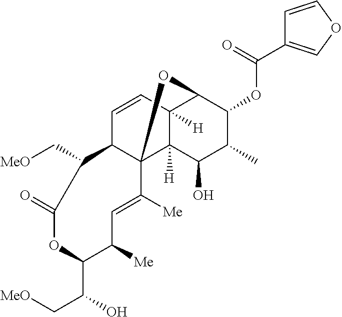 [SMILES: COC[C@@H](O)[C@H]1OC(=O)[C@H](COC)[C@H]2C=C[C@H]3[C@H]4O[C@]2(/C(C)=C/[C@H]1C)[C@@H]3[C@H](O)[C@@H](C)[C@H]4OC(=O)c1ccoc1] | 8-O-Furane-3′- carbonylbaleomycin | 546.6 | 569.2 (+Na⁺) |
| 8 | 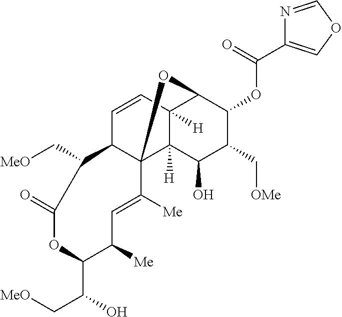 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1cocn1] | 8-O-Oxazole-4′- carbonylbranimycin | 577.6 | 560.5 (−H₂O) |
| 9 | 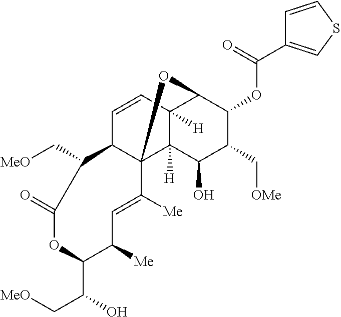 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1ccsc1] | 8-O-Thiophene-3′- carbonylbranimycin | 592.7 | 575.4 (−H₂O) |
| 10 | 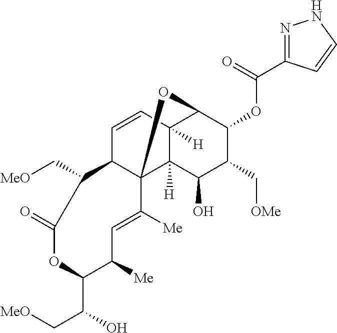 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1cc[nH]n1] | 8-O-1H-Pyrazole-3′- carbonylbranimycin | 576.7 | 559.5 |
| 11 | 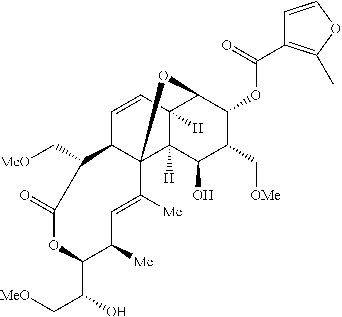 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1ccoc1C] | 8-O-2-Methylfurane- 3′- carbonylbranimycin | 590.7 | 573.5 (−H₂O) |
| 12 | 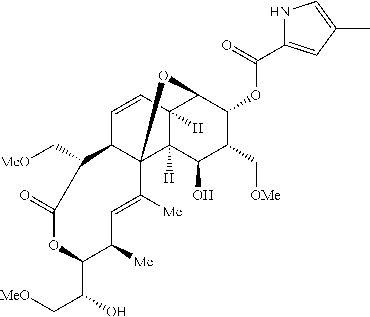 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1cc(C)c[nH]1] | 8-O-4-Methyl-1H- pyrrole-2′- carbonylbranimycin | 589.7 | 572.5 (−H₂O) |
| 13 | 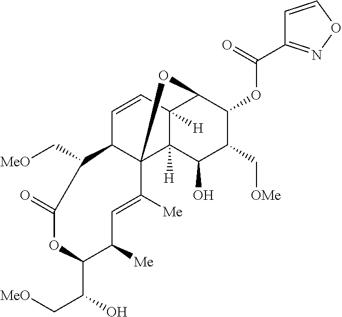 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1ccon1] | 8-O-Isoxazole-3′- carbonylbranimycin | 577.6 | 560.5 (−H₂O) |
| 14 | 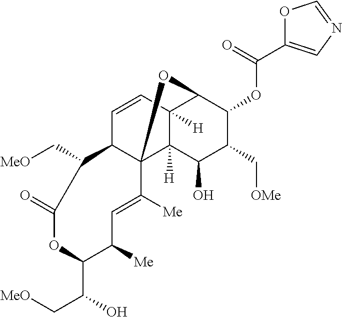 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1cnco1] | 8-O-Oxazole-5′ carbonylbranimycin | 577.6 | 560.5 (−H₂O) |
| 15 | 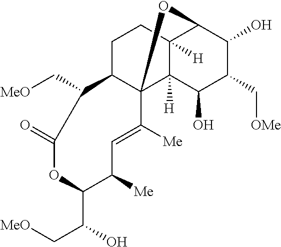 [SMILES: COC[C@H]1[C@@H](O)[C@@H]2O[C@@]34/C(C)=C/[C@@H](C)[C@@H]([C@H](O)COC)OC(=O)[C@H](COC)[C@H]3CC[C@@H]2[C@H]4[C@@H]1O] | 4,5-Dihydro- branimycin | 484.6 | 485.3 and 467.3 (−H₂O) |
| 16 | 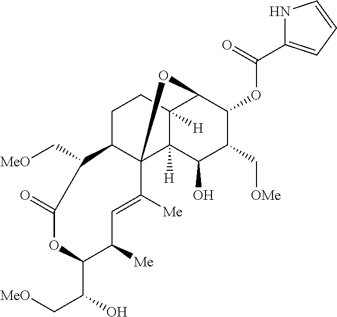 [SMILES: COC[C@@H]1[C@@H](O)[C@@H]2[C@H]3CC[C@@H]4[C@@H](COC)C(=O)O[C@H]([C@H](O)COC)[C@H](C)/C=C(\C)[C@@]24O[C@H]3[C@@H]1OC(=O)c1ccc[nH]1] | 4,5-Dihydro-8-O-1H- pyrrole-2′- carbonylbranimycin | 577.7 | 560.4 (−H₂O) |
| 17 | 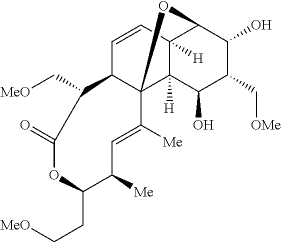 [SMILES: COCC[C@H]1OC(=O)[C@H](COC)[C@H]2C=C[C@H]3[C@H]4O[C@]2(/C(C)=C/[C@H]1C)[C@@H]3[C@H](O)[C@@H](COC)[C@H]4O] | 17-Deoxybranimycin | 466.6 | 449.3 (−H₂O) |
| 18 | 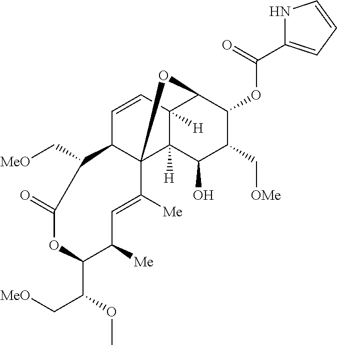 [SMILES: COC[C@@H]1[C@@H](O)[C@@H]2[C@H]3C=C[C@@H]4[C@@H](COC)C(=O)O[C@H]([C@@H](COC)OC)[C@H](C)/C=C(\C)[C@@]24O[C@H]3[C@@H]1OC(=O)c1ccc[nH]1] | 17-O-Methyl-8-O- 1H-pyrrole-2′- carbonylbranimycin | 589.7 | 572.2 |
| 19 | 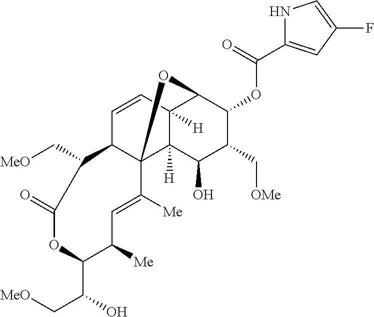 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1cc(F)c[nH]1] | 8-O-4-Fluoro-1H- pyrrole-2′- carbonylbranimycin | 593.7 | 576.6 (−H₂O) |
| 20 | 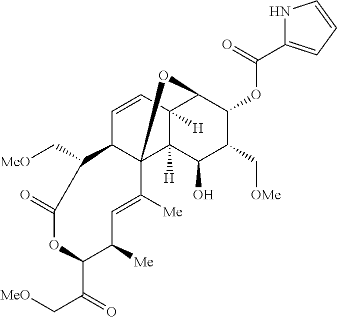 [SMILES: COCC(=O)[C@H]1OC(=O)[C@H](COC)[C@H]2C=C[C@H]3[C@H]4O[C@]2(/C(C)=C/[C@H]1C)[C@@H]3[C@H](O)[C@@H](COC)[C@H]4OC(=O)c1ccc[nH]1] | 18-Deoxy-18-oxo-8- O-1H-pyrrole-2′- carbonylbranimycin | 573.7 | 556.5 (−H₂O) |
| 21 | 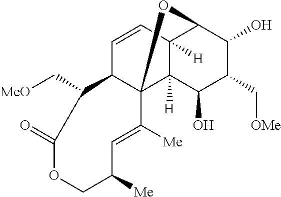 [SMILES: COC[C@H]1[C@@H](O)[C@@H]2O[C@@]34/C(C)=C/[C@@H](C)COC(=O)[C@H](COC)[C@H]3C=C[C@@H]2[C@H]4[C@@H]1O] | 17,18-Dinor- branimycin | 408.5 | ND |
| 22 | 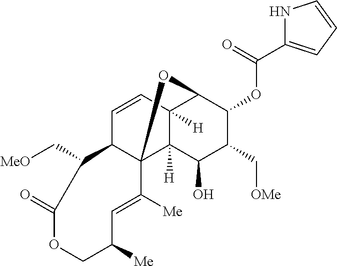 [SMILES: COC[C@@H]1[C@@H](O)[C@@H]2[C@H]3C=C[C@@H]4[C@@H](COC)C(=O)OC[C@H](C)/C=C(\C)[C@@]24O[C@H]3[C@@H]1OC(=O)c1ccc[nH]1] | 17,18-Dinor-8-O-1H- pyrrole-2′- carbonybranimycin | 501.6 | 484.2 (−H₂O) |
| 23 | 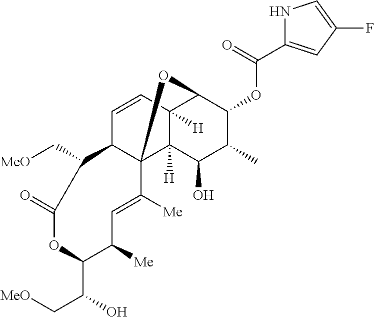 [SMILES: COC[C@@H](O)[C@H]1OC(=O)[C@H](COC)[C@H]2C=C[C@H]3[C@H]4O[C@]2(/C(C)=C/[C@H]1C)[C@@H]3[C@H](O)[C@@H](C)[C@H]4OC(=O)c1cc(F)c[nH]1] | 8-O-4-Fluoro-1H- pyrrole-2′- carbonylbaleomycin | 563.6 | 546.2 (−H₂O) |
| 24 | 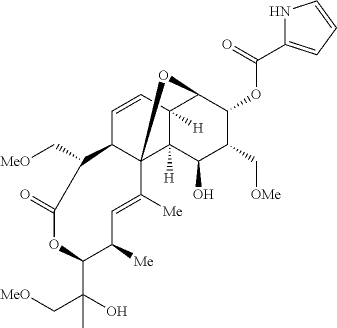 [SMILES: COC[C@@H]1[C@@H](O)[C@@H]2[C@H]3C=C[C@@H]4[C@@H](COC)C(=O)O[C@H](C(C)(O)COC)[C@H](C)/C=C(\C)[C@@]24O[C@H]3[C@@H]1OC(=O)c1ccc[nH]1] | 17-Methyl-8-O-1H- pyrrole-2′- carbonylbranimycin | 589.7 | 572.4 (−H₂O) |
| 25 | 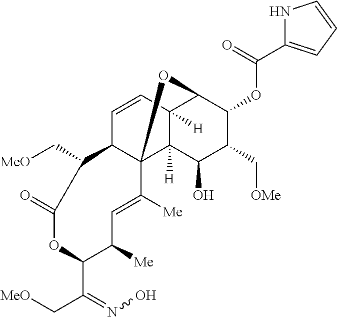 [SMILES: COCC(=NO)[C@H]1OC(=O)[C@H](COC)[C@H]2C=C[C@H]3[C@H]4O[C@]2(/C(C)=C/[C@H]1C)[C@@H]3[C@H](O)[C@@H](COC)[C@H]4OC(=O)c1ccc[nH]1] | 18-Deoxy-18- oximino-8-O-1H- pyrrole-2′- carbonylbranimycin | 588.7 | 589.4 |
| 26 | 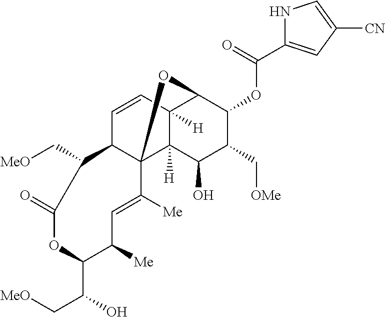 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1cc(C#N)c[nH]1] | 8-O-4-Cyano-1H- pyrrole-2′- carbonylbranimycin | 600.7 | 599.6 |
| 27 | 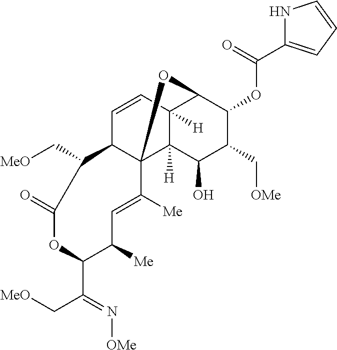 [SMILES: COC/C(=N\OC)[C@H]1OC(=O)[C@H](COC)[C@H]2C=C[C@H]3[C@H]4O[C@]2(/C(C)=C/[C@H]1C)[C@@H]3[C@H](O)[C@@H](COC)[C@H]4OC(=O)c1ccc[nH]1] | 18-Deoxy-18- methoximino-8-O- 1H-pyrrole-2′- carbonylbranimycin | 602.7 | 603.6 |
| 28 | 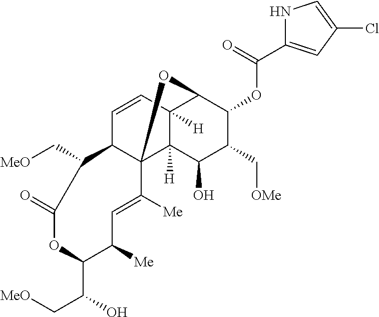 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1cc(Cl)c[nH]1] | 8-O-4-Chloro-1H- pyrrole-2′- carbonylbranimycin | 610.1 | 592.5 (−H₂O) |
| 29 | 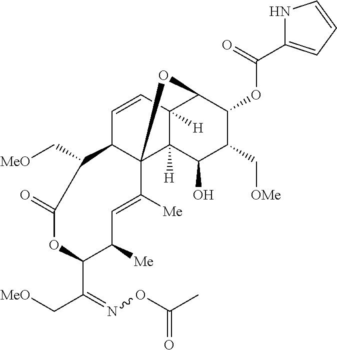 [SMILES: COCC(=NOC(C)=O)[C@H]1OC(=O)[C@H](COC)[C@H]2C=C[C@H]3[C@H]4O[C@]2(/C(C)=C/[C@H]1C)[C@@H]3[C@H](O)[C@@H](COC)[C@H]4OC(=O)c1ccc[nH]1] | 18-Acetylimino-18- deoxy-8-O-1H- pyrrole-2′- carbonylbranimycin | 630.7 | 631.6 |
| 30 | 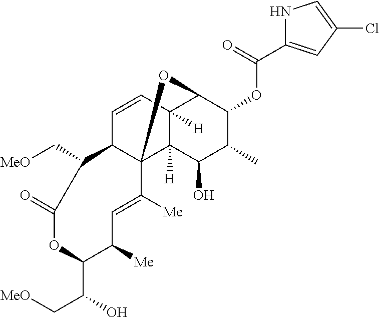 [SMILES: COC[C@@H](O)[C@H]1OC(=O)[C@H](COC)[C@H]2C=C[C@H]3[C@H]4O[C@]2(/C(C)=C/[C@H]1C)[C@@H]3[C@H](O)[C@@H](C)[C@H]4OC(=O)c1cc(Cl)c[nH]1] | 8-O-4-Chloro-1H- pyrrole-2′- carbonylbaleomycin | 580.1 | 562.5 (−H₂O) |
| 31 | 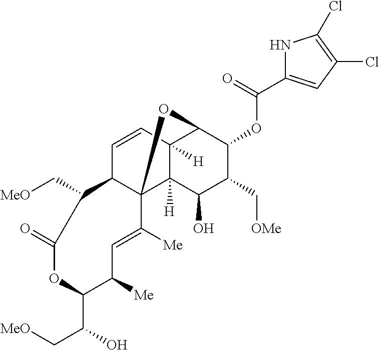 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1cc(Cl)c(Cl)[nH]1] | 8-O-4,5-Dichloro- 1H-pyrrole-2′- carbonylbranimycin | 644.6 | 626.6 (−H₂O) |
| 32 | 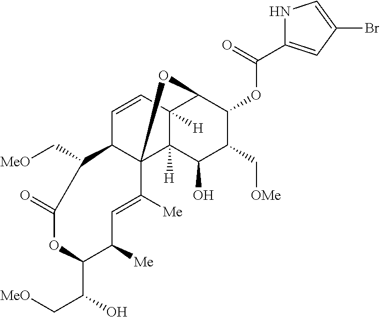 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1cc(Br)c[nH]1] | 8-O-4-Bromo-1H- pyrrole-2′- carbonylbranimycin | 654.6 | 638.5 (−H₂O) |
| 33 | 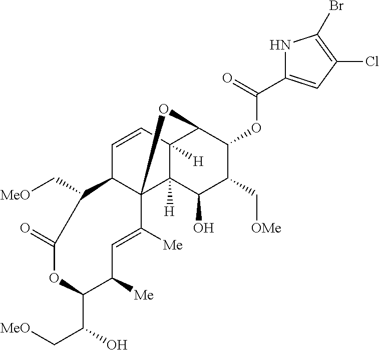 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1cc(Cl)c(Br)[nH]1] | 8-O-5-Bromo-4- Chloro-1H-pyrrole- 2′- carbonylbranimycin | 803.3 | 802.3 (−H) |
| 34 | 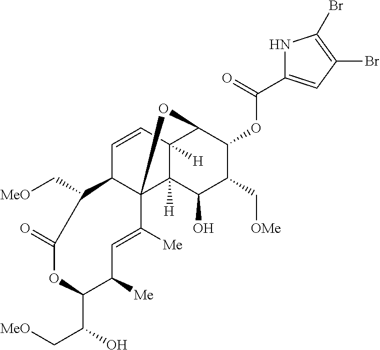 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1cc(Br)c(Br)[nH]1] | 8-O-4,5-Dibromo- 1H-pyrrole-2′- carbonylbranimycin | 733.4 | 732.3 (−H) |
| 35 | 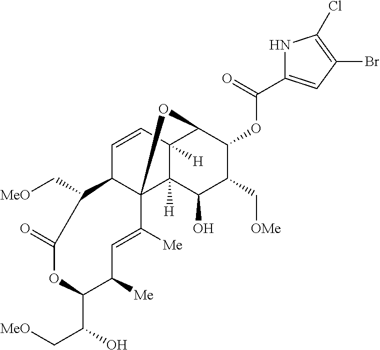 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1cc(Br)c(Cl)[nH]1] | 8-O-4-Bromo-5- Chloro-1H-pyrrole- 2′- carbonylbranimycin | 803.3 | 802.3 (−H) |
| 36 | 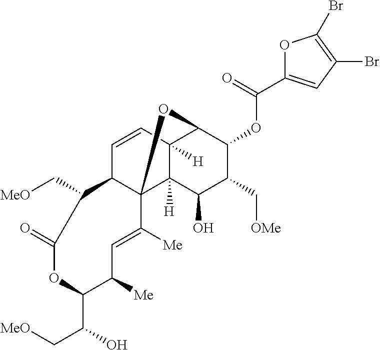 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1cc(Br)c(Br)o1] | 8-O-4,5- Dibromofurane-2′- carbonylbranimycin | 734.5 | 779.3 (+HCOOH) |
| 37 | 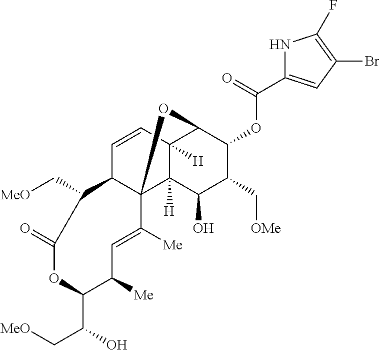 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1cc(Br)c(F)[nH]1] | 8-O-4-Bromo-5- Fluoro-1H-pyrrole- 2′- carbonylbranimycin | 672.6 | 672.4 |
| 38 | 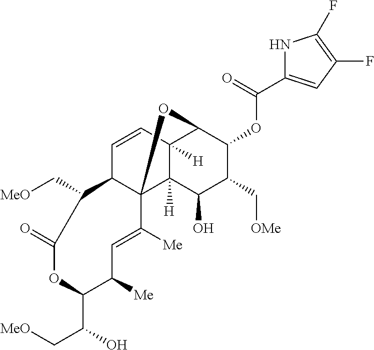 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1cc(F)c(F)[nH]1] | 8-O-4,5-Difluoro-1H- pyrrole-2′- carbonylbranimycin | 611.6 | 610.6 |
| 39 | 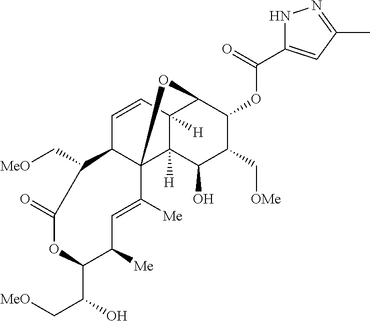 [SMILES: COC[C@@H]1[C@@H](O)[C@@H]2[C@H]3C=C[C@@H]4[C@@H](COC)C(=O)O[C@H]([C@H](O)COC)[C@H](C)/C=C(\C)[C@@]24O[C@H]3[C@@H]1OC(=O)c1cc(C)n[nH]1] | 8-O-5-Methyl-1H- Pyrazole-3′- carbonylbranimycin | 590.7 | 591.5 |
| 40 | 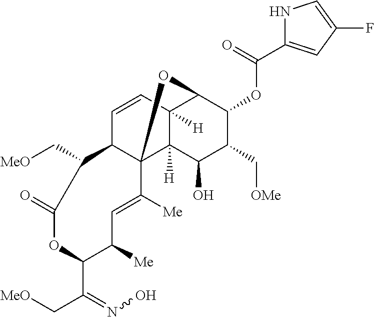 [SMILES: COCC(=NO)[C@H]1OC(=O)[C@H](COC)[C@H]2C=C[C@H]3[C@H]4O[C@]2(/C(C)=C/[C@H]1C)[C@@H]3[C@H](O)[C@@H](COC)[C@H]4OC(=O)c1cc(F)c[nH]1] | 18-Deoxy-18- oximino-8-O-4- Fluoro-1H-pyrrole- 2′- carbonylbranimycin | 606.7 | 607.5 |
| 41 | 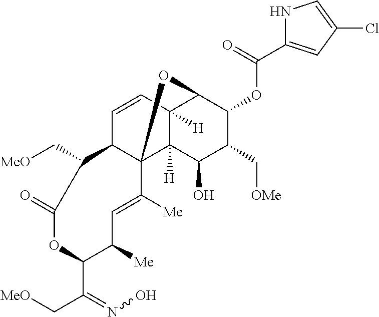 [SMILES: COCC(=NO)[C@H]1OC(=O)[C@H](COC)[C@H]2C=C[C@H]3[C@H]4O[C@]2(/C(C)=C/[C@H]1C)[C@@H]3[C@H](O)[C@@H](COC)[C@H]4OC(=O)c1cc(Cl)c[nH]1] | 18-Deoxy-18- oximino-8-O-4- Chloro-1H-pyrrole- 2′- carbonylbranimycin | 623.1 | 623.5 |
| 42 | 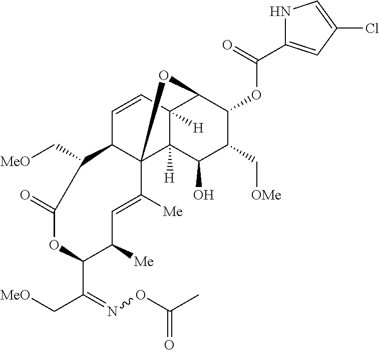 [SMILES: COCC(=NOC(C)=O)[C@H]1OC(=O)[C@H](COC)[C@H]2C=C[C@H]3[C@H]4O[C@]2(/C(C)=C/[C@H]1C)[C@@H]3[C@H](O)[C@@H](COC)[C@H]4OC(=O)c1cc(Cl)c[nH]1] | 18-Acetylimino-18- deoxy-8-O-4-Chloro- 1H-pyrrole-2′- carbonylbranimycin | 665.1 | 665.5 |

TABLE 2
NMR Data of Representative Compounds of the Invention

| Cpd # | NMR Data |
|---|---|
| 1 | ¹H NMR (300 MHz, CDCl₃) δ ppm 6.05-6.10 (1 H, m), 5.74 (1H, d), 5.42 (1 H, |
| | dd), 5.07 (1 H, dd), 4.17 (1 H, d), 4.05 (1 H, dt), 3.88 (1 H, t), 3.57-3.62 (3 H, m), |
| | 3.50-3.53 (1 H, m), 3.42-3.46 (4 H, m), 3.35 (3 H, s), 3.06-3.08 (2 H, m), |
| | 2.88-2.94 (1 H, ddd), 2.67 (1 H, d), 2.50 (1 H, d), 2.13-2.20 (1 H, m), 1.72 (3 H, s), 1.31 (3 H, |
| | d), 1.06 (3 H, d). |
| 2 | ¹H NMR (300 MHz, CDCl₃) δ ppm 9.11 (1 H, br. s.), 6.99-7.05 (1 H, m), |
| | 6.87-6.92 (1 H, m), 6.28-6.33 (1 H, m), 5.94-6.03 (1 H, m), 5.72 (1 H, d), 5.41 (1 H, |
| | dd), 5.25 (1 H, t), 5.06 (1 H, dd), 4.27 (1 H, d), 4.05-4.15 (2 H, m), 3.39-3.63 (10 |
| | H, m), 3.33 (3 H, s), 3.30 (3 H, s), 3.01-3.13 (2 H, m), 2.85-2.99 (1 H, m), |
| | 2.68-2.79 (1 H, m), 2.54-2.61 (2 H, m), 2.51 (1 H, d), 1.73 (3 H, s), 1.24-1.35 (3 H, |
| | m). |
| 3 | ¹H NMR (300 MHz, CDCl₃) δ ppm 9.20 (1 H, br. s.), 7.01 (1 H, td), 6.90 (1 H, |
| | ddd), 6.30 (1 H, dt), 5.95-6.06 (1 H, m), 5.77 (1 H, dd), 5.41 (1 H, dd), 5.19 (1 H, |
| | t), 5.06 (1 H, dd), 4.28 (1 H, d), 4.10 (1 H, ddt), 3.65-3.75 (1 H, m), 3.37-3.63 (7 |
| | H, m), 3.29-3.36 (3 H, m), 3.04-3.13 (2 H, m), 2.91 (1 H, sxt), 2.53-2.66 (3 H, |
| | m), 2.34-2.49 (1 H, m), 1.72 (3 H, s), 1.54 (1 H, d), 1.31 (3 H, d), 0.97-1.04 (3 H, |
| | m). |
| 4 | ¹H NMR (300 MHz, CDCl₃) δ ppm 9.16 (1 H, br. s.), 6.90-7.00 (1 H, m), |
| | 6.87-6.92 (1 H, m), 6.28-6.30 (1 H, m), 5.86-5.95 (1 H, m), 5.70 (1 H, d), 5.44 (1 H, |
| | dd), 5.24-5.41 (2 H, m), 4.27 (1 H, d), 4.02-4.05 (2 H, m), 3.31-3.53 (7 H, m), |
| | 3.35 (3 H, s), 3.32 (3 H, s), 3.28 (3 H, s), 2.86-3.05 (2 H, m), 2.44-2.78 (4H, m), |
| | 1.72-2.01 (2 H, m), 1.71 (3 H, s), 1.12-1.30 (2H, m), 1.00-1.12 (3 H, m). |
| 5 | ¹H NMR (300 MHz, CDCl₃) δ ppm 7.93-8.07 (1 H, m), 7.42-7.56 (1 H, m), |
| | 6.67-6.77 (1 H, m), 5.99 (1 H, ddd), 5.72 (1 H, dd), 5.41 (1 H, dd), 5.26 (1 H, t), |
| | 4.99-5.12 (1 H, m), 4.26 (1 H, d), 4.01-4.14 (2 H, m), 3.40-3.63 (10H, m), |
| | 3.28-3.34 (6 H, m), 3.00-3.11 (2 H, m), 2.86-2.97 (1 H, m), 2.68-2.81 (1 H, m), |
| | 2.50-2.57 (3 H, m), 1.72 (3 H, d), 1.30 (3 H, d). |
| 6 | ¹H NMR (300 MHz, CDCl₃) δ ppm 8.94 (1 H, br. s.), 6.78 (1 H, dt), 5.96-5.98 (2 |
| | H, m), 5.69 (1 H, d), 5.44 (1 H, dd), 5.21 (1 H, t), 5.04 (1 H, dd), 4.24 (1 H, d), |
| | 4.05-4.10 (2 H, m), 3.39-3.63 (10 H, m), 3.31 (3 H, s), 3.28 (3 H, s), 3.01-3.13 (2 H, |
| | m), 2.85-2.99 (1 H, m), 2.68-2.79 (1 H, m), 2.54-2.61 (3 H, m), 2.31 (3 H, s), |
| | 1.71 (3 H, s), 1.27-1.30 (3 H, m). |
| 7 | ¹H NMR (300 MHz, CDCl₃) δ ppm 8.02 (1 H, d), 7.46 (1 H, t), 6.71-6.77 (1 H, |
| | m), 5.95-6.06 (1 H, m), 5.78 (1 H, d), 5.38-5.45 (1 H, m), 5.19 (1 H, t), 5.06 (1 |
| | H, dd), 4.28 (1 H, d), 4.10 (1 H, ddt), 3.38-3.70 (8 H, m), 3.33 (3 H, s), |
| | 3.05-3.11 (2 H, m), 2.86-2.95 (1 H, m), 2.51-2.59 (3 H, m), 2.34-2.47 (1 H, m), 1.72 (3 H, |
| | s), 1.48 (1 H, d), 1.31 (3 H, d), 0.99 (3 H, d). |
| 8 | ¹H NMR (300 MHz, CDCl₃) δ ppm 8.27 (1 H, d), 7.96 (1 H, d), 5.99 (1 H, ddd), |
| | 5.72 (1 H, d), 5.41 (1 H, dd), 5.35 (1 H, t), 5.06 (1 H, dd), 4.28 (1 H, d), |
| | 4.05-4.14 (2 H, m), 3.36-3.62 (10 H, m), 3.32 (3 H, s), 3.29 (3 H, s), 3.01-3.12 (2 H, m), |
| | 2.87-2.96 (1 H, m), 2.69-2.81 (1 H, m), 2.52-2.62 (3 H, m), 1.72 (3 H, s), |
| | 1.30 (3 H, d). |
| 9 | ¹H NMR (300 MHz, CDCl₃) δ ppm 8.09 (1 H, dd), 7.50 (1 H, dd), 7.36 (1 H, dd), |
| | 5.92-6.02 (1 H, m), 5.73 (1 H, d), 5.41 (1 H, dd), 5.28 (1 H, t), 5.06 (1 H, dd), |
| | 4.28 (1 H, d), 4.02-4.16 (2 H, m), 3.39-3.62 (10 H, m), 3.33 (3 H, s), 3.29 (3 H, s), |
| | 3.02-3.13 (2 H, m), 2.87-2.97 (1 H, m), 2.71-2.82 (1 H, m), 2.53-2.61 (3 H, |
| | m), 1.72 (3 H, s), 1.31 (3 H, d). |
| 10 | ¹H NMR (300 MHz, CDCl₃) δ ppm 7.79 (1 H, d), 6.82 (1 H, d), 5.99 (1 H, t), |
| | 5.74 (1 H, d), 5.31-5.47 (2 H, m), 5.07 (1 H, dd), 4.33 (1 H, d), 4.12 (2 H, d), |
| | 3.38-3.64 (10 H, m), 3.33 (3 H, s), 3.29 (3 H, s), 3.01-3.16 (2 H, m), 2.85-3.00 (1 H, |
| | m), 2.69-2.83 (1 H, m), 2.51-2.66 (3 H, m), 1.73 (3 H, s), 1.31 (3 H, d). |
| 11 | ¹H NMR (300 MHz, CDCl₃) δ ppm 7.27 (1 H, d), 6.61 (1 H, d), 5.99 (1 H, t), |
| | 5.73 (1 H, d), 5.42 (1 H, dd), 5.27 (1 H, t), 5.07 (1 H, dd), 4.26 (1 H, d), 3.99-4.16 (2 H, |
| | m), 3.43-3.63 (7 H, m), 3.42 (3 H, s), 3.34 (3 H, s), 3.31 (3 H, s), 3.03-3.13 (2 H, |
| | m), 2.93 (1 H, sxt), 2.68-2.82 (1 H, m), 2.60 (3 H, s), 2.55 (3 H, d), 1.73 (3 H, s), |
| | 1.32 (3 H, d). |
| 12 | ¹H NMR (300 MHz, CDCl₃) δ ppm 8.98 (1 H, br. s.), 6.74-6.82 (1 H, m), |
| | 6.66-6.74 (1 H, m), 5.98 (1 H, t), 5.72 (1 H, d), 5.40 (1 H, dd), 5.20-5.27 (1 H, m), |
| | 5.06 (1 H, dd), 4.25 (1 H, d), 4.08 (2 H, d), 3.43-3.63 (7 H, m), 3.41 (3 H, s), 3.33 (3 H, |
| | s), 3.30 (3 H, s), 3.01-3.12 (2 H, m), 2.91 (1 H, sxt), 2.72 (1 H, tt), 2.49-2.62 (3 |
| | H, m), 2.13 (3 H, s), 1.72 (3 H, s), 1.26-1.34 (3 H, m). |
| 13 | ¹H NMR (300 MHz, CDCl₃) δ ppm 8.56 (1 H, d), 6.79 (1 H, d), 5.94-6.07 (1 H, |
| | m), 5.72 (1 H, d), 5.33-5.48 (2 H, m), 4.99-5.14 (1 H, m), 4.30 (1 H, d), |
| | 4.01-4.16 (2 H, m), 3.38-3.64 (9 H, m), 3.33 (3 H, s), 3.29 (3 H, s), 3.25 (1 H, d), |
| | 2.99-3.13 (2 H, m), 2.84-2.97 (1 H, m), 2.68-2.82 (1 H, m), 2.64 (1 H, d), |
| | 2.49-2.60 (2 H, m), 1.72 (3 H, s), 1.31 (3 H, d). |
| 14 | ¹H NMR (300 MHz, CDCl₃) δ ppm 8.05 (1 H, s), 7.78 (1 H, s), 5.99 (1 H, ddd), |
| | 5.71 (1 H, dd), 5.43 (1 H, dd), 5.33 (1 H, t), 5.06 (1 H, dd), 4.28 (1 H, d), |
| | 4.00-4.16 (2 H, m), 3.39-3.62 (9 H, m), 3.31-3.33 (4 H, m), 3.27-3.31 (3 H, m), |
| | 3.01-3.14 (2 H, m), 2.91 (1 H, sxt), 2.68-2.82 (1 H, m), 2.56 (3 H, d), 1.68-1.75 (3 H, |
| | m), 1.30 (3 H, d). |
| 15 | ¹H NMR (300 MHz, CDCl₃) δ ppm 5.52 (1 H, d), 5.04 (1 H, dd), 4.04-4.20 (5 H, |
| | m), 3.93-3.99 (1 H, m), 3.78 (1 H, dd), 3.56-3.69 (2 H, m), 3.37-3.51 (9 H, m), |
| | 3.30-3.31 (3H, m), 3.03-3.05 (1H, m), 2.83-2.89 (1 H, m), 2.61-2.79 (1 H, m), |
| | 2.49 (1 H, d), 2.38-2.43 (1H, m), 2.33-2.55 (1 H, m), 2.14-2.22 (2H, m), |
| | 2.08-2.05 (3H, m), 1.77 (3 H, s), 1.51-1.61 (5 H, m), 1.29 (3 H, d). |
| 16 | ¹H NMR (300 MHz, CDCl₃) δ ppm 9.09 (1 H, s), 7.01 (1 H, td), 6.87 (1 H, ddd), |
| | 6.29 (1 H, dt), 5.55 (1 H, d), 5.25 (1 H, t), 5.06 (1 H, dd), 4.28 (1 H, d), |
| | 3.95-4.11 (1 H, m), 3.51-3.65 (3 H, m), 3.36-3.50 (7 H, m), 3.30 (6 H, d), 2.89 (1 H, q), |
| | 2.61-2.79 (2 H, m), 2.49 (1 H, d), 2.13-2.31 (3 H, m), 1.77 (3 H, s), 1.51-1.61 (5 |
| | H, m), 1.29 (3 H, d). |
| 17 | ¹H NMR (300 MHz, CDCl₃) δ ppm 6.06 (1 H, ddd), 5.68 (1 H, d), 5.43 (1 H, dd), |
| | 5.23-5.35 (1 H, m), 3.94-4.20 (3 H, m), 3.71-3.84 (1 H, m), 3.42-3.69 (4 H, |
| | m), 3.30-3.41 (10 H, m), 2.95-3.09 (3 H, m), 2.67-2.85 (2 H, m), 2.43-2.52 (1 |
| | H, m), 2.23-2.36 (1 H, m), 2.10 (1 H, d), 1.94-2.08 (1 H, m), 1.78-1.93 (1 H, |
| | m), 1.71 (3 H, d), 1.05-1.15 (3 H, m). |
| 18 | ¹H NMR (300 MHz, CDCl₃) δ ppm 9.25 (1 H, br. s.), 6.97-7.06 (1 H, m), |
| | 6.81-6.93 (1 H, m), 6.30 (1 H, dt), 5.98 (1 H, ddd), 5.72 (1 H, dd), 5.41 (1 H, dd), |
| | 5.13-5.31 (2 H, m), 4.21-4.33 (1 H, m), 4.08 (1 H, dd), 3.73 (1 H, dd), 3.45-3.66 (5 H, |
| | m), 3.42 (1 H, s), 3.36 (3 H, s), 3.32 (3 H, s), 3.27-3.30 (3 H, m), 2.98-3.14 (2 H, |
| | m), 2.82-2.97 (1 H, m), 2.66-2.79 (1 H, m), 2.50-2.63 (2 H, m), 1.64-1.78 (3 |
| | H, m), 1.21 (3 H, d). |
| 19 | ¹H NMR (300 MHz, CDCl₃) δ ppm 9.16 (1 H, br. s.), 6.71-6.91 (1 H, m), 6.57 (1 |
| | H, d), 5.89-6.14 (1 H, m), 5.71 (1 H, d), 5.33-5.55 (1 H, m), 5.14-5.32 (1 H, m), |
| | 4.91-5.13 (1 H, m), 4.25 (1 H, d), 3.97-4.20 (2 H, m), 3.37-3.63 (10 H, m), |
| | 3.31-3.36 (3 H, m), 3.29 (3 H, s), 2.99-3.13 (2 H, m), 2.91 (1 H, sxt), 2.60-2.82 (2 H, |
| | m), 2.47-2.60 (2 H, m), 1.71 (3 H, s), 1.29 (3 H, d). |
| 20 | ¹H NMR (300 MHz, CDCl₃) δ ppm 9.23 (1 H, br. s.), 7.03 (1 H, td), 6.89 (1 H, |
| | ddd), 6.31 (1 H, dt), 6.02 (1 H, ddd), 5.70 (2 H, d), 5.44 (1 H, dd), 5.18-5.32 (1 H, |
| | m), 4.24-4.36 (3 H, m), 4.04-4.19 (1 H, m), 3.43-3.69 (8 H, m), 3.32-3.40 (3 |
| | H, m), 3.30 (3 H, s), 3.04-3.24 (3 H, m), 2.51-2.75 (3 H, m), 1.70 (3 H, d), |
| | 1.12 (3 H, d). |
| 22 | ¹H NMR (300 MHz, CDCl₃) δ ppm 9.24 (1 H, br. s.), 7.02 (1 H, td), 6.84-6.94 (1 |
| | H, m), 6.30 (1 H, dt), 5.90-6.09 (1 H, m), 5.47 (1 H, d), 5.19-5.33 (1 H, m), |
| | 4.33 (1 H, br. s.), 3.96-4.16 (1 H, m), 3.45-3.71 (4 H, m), 3.31-3.42 (5 H, m), |
| | 3.24-3.31 (3 H, m), 2.77 (4 H, d), 2.51-2.64 (2 H, m), 1.71 (3 H, d), 1.66 (2 H, s), |
| | 1.18 (3 H, d). |
| 23 | ¹H NMR (300 MHz, CDCl₃) δ ppm 8.99 (1 H, br. s.), 6.74-6.77 (1 H, m), |
| | 6.55-6.58 (1 H, m), 6.05-6.10 (1 H, m), 5.74 (1H, dd), 5.42 (1 H, dd), 5.16 (1H, t), |
| | 5.05 (1 H, dd), 4.24 (1 H, d), 4.08 (1 H, dt), 3.37-3.70 (8 H, m), 3.31 (3 H, s), |
| | 3.02-3.08 (2 H, m), 2.83-2.94 (1 H, ddd), 2.52-2.65 (3 H, m), 2.34-2.47 (1 H, m), 1.58-1.74 (4 |
| | H, m), 1.30 (3 H, d), 0.98 (3 H, d). |
| 24 | ¹H NMR (300 MHz, CDCl₃) δ ppm 9.23 (1 H, br. s.), 7.02 (1 H, dd), 6.89 (1 H, d), |
| | 6.24-6.34 (1 H, m), 5.98 (1 H, t), 5.79 (1 H, d), 5.43 (1 H, d), 5.18-5.27 (1 H, m), |
| | 4.92-5.08 (1 H, m), 4.20-4.32 (1 H, m), 4.09 (1 H, d), 3.43-3.63 (5 H, m), |
| | 3.26-3.42 (12 H, m), 3.12 (2 H, br. s.), 2.90 (1 H, q), 2.65-2.80 (1 H, m), 2.49-2.63 (2 |
| | H, m), 1.73 (3 H, s), 1.32-1.43 (6 H, m). |
| 25 | ¹H NMR (300 MHz, CDCl₃) δ ppm 9.68 (1 H, br. s.), 7.03 (1 H, d), 6.83-6.95 (1 |
| | H, m), 6.41 (1 H, d), 6.23-6.36 (1 H, m), 5.91-6.09 (1 H, m), 5.71 (1 H, d), |
| | 5.44 (1 H, d), 5.16-5.32 (1 H, m), 4.30 (1 H, d), 4.04-4.20 (2 H, m), 3.40-3.66 (8 H, |
| | m), 3.19-3.37 (7 H, m), 3.03-3.18 (2 H, m), 2.72 (1 H, tt), 2.51-2.65 (2 H, m), |
| | 1.76 (5 H, s), 1.10 (3 H, d). |
| 26 | ¹H NMR (400 MHz, CDCl₃) δ ppm 9.59 (1 H, br. s.), 7.40-7.49 (1 H, m), 7.10 (1 |
| | H, s), 5.99 (1 H, t), 5.71 (1 H, d), 5.37-5.50 (1 H, m), 5.27 (1 H, t), 5.07 (1 H, dd), |
| | 4.22-4.37 (1 H, m), 3.96-4.17 (2 H, m), 3.38-3.63 (10 H, m), 3.33 (3 H, s), |
| | 3.30 (3 H, s), 3.20-3.28 (1 H, m), 3.00-3.14 (2 H, m), 2.86-3.00 (1 H, m), 2.74 (1 H, |
| | tt), 2.48-2.60 (2 H, m), 1.67-1.76 (3 H, m), 1.26-1.37 (3 H, m). |
| 27 | ¹H NMR (400 MHz, CDCl₃) δ ppm 9.14-9.32 (1 H, m), 6.99-7.07 (1 H, m), |
| | 6.86-6.93 (1 H, m), 6.26-6.36 (2 H, m), 5.95-6.08 (1 H, m), 5.69 (1 H, d), |
| | 5.40-5.52 (1 H, m), 5.18-5.30 (1 H, m), 4.26-4.33 (1 H, m), 4.04-4.20 (3 H, m), |
| | 3.89-3.97 (3 H, m), 3.41-3.63 (8 H, m), 3.30-3.36 (3 H, m), 3.26-3.31 (3 H, m), |
| | 3.14-3.23 (1 H, m), 3.03-3.13 (2 H, m), 2.67-2.77 (1 H, m), 2.52-2.64 (2 H, m), |
| | 1.68-1.81 (3 H, m), 1.00-1.09 (3 H, m). |
| 28 | ¹H NMR (300 MHz, CDCl₃) δ ppm 9.14 (1 H, br. s.), 6.89-7.04 (1 H, m), 6.78 (1 |
| | H, dd), 5.91-6.08 (1 H, m), 5.65-5.81 (1 H, m), 5.42 (1 H, dd), 5.24 (1 H, t), |
| | 5.06 (1 H, dd), 4.25 (1 H, d), 3.95-4.17 (2 H, m), 3.38-3.65 (10 H, m), 3.32 (7 H, d), |
| | 3.00-3.14 (2 H, m), 2.84-3.00 (1 H, m), 2.66-2.81 (1 H, m), 2.50-2.60 (2 H, |
| | m), 1.72 (3 H, s), 1.30 (3 H, d). |
| 29 | ¹H NMR (300 MHz, CDCl₃) δ ppm 9.65 (1 H, br. s.), 7.04 (1 H, br. s.), 6.90 (1 H, |
| | br. s.), 6.24-6.48 (2 H, m), 5.96-6.08 (1 H, m), 5.66-5.82 (1 H, m), |
| | 5.36-5.56 (1 H, m), 5.13-5.31 (1 H, m), 4.04-4.42 (4 H, m), 3.40-3.67 (8 H, m), |
| | 3.22-3.36 (7 H, m), 3.00-3.21 (3 H, m), 2.65-2.82 (1 H, m), 2.50-2.65 (2 H, m), |
| | 2.15-2.29 (2 H, m), 1.76 (3 H, s), 0.99-1.17 (3 H, m). |
| 30 | ¹H NMR (300 MHz, CDCl₃) δ ppm 9.26 (1 H, br. s.), 6.87-7.00 (1 H, m), 6.79 (1 |
| | H, dd), 5.92-6.12 (1 H, m), 5.64-5.84 (1 H, m), 5.41 (1 H, d), 5.13-5.23 (1 H, |
| | m), 4.97-5.12 (1 H, m), 4.26 (1 H, m), 4.01-4.15 (1 H, m), 3.37-3.74 (8 H, m), |
| | 3.33 (3 H, s), 3.02-3.13 (2 H, m), 2.80-2.98 (1 H, m), 2.51-2.62 (2 H, m), |
| | 2.33-2.48 (1 H, m), 1.66-1.75 (3 H, m), 1.26-1.34 (3 H, m), 0.95-1.02 (3 H, m). |
| 31 | ¹H NMR (300 MHz, CDCl₃) δ ppm 9.44 (1 H, br. s.), 6.72-6.89 (1 H, m), |
| | 5.90-6.09 (1 H, m), 5.71 (1 H, dd), 5.42 (1 H, dd), 5.23 (1 H, t), 5.06 (1 H, dd), 4.26 (1 |
| | H, d), 3.94-4.16 (2 H, m), 3.38-3.68 (9 H, m), 3.24-3.37 (6 H, m), 3.00-3.15 (2 |
| | H, m), 2.91 (1 H, sxt), 2.62-2.85 (1 H, m), 2.43-2.62 (2 H, m), 1.57-1.80 (3 H, |
| | m), 1.21-1.35 (5 H, m). |
| 32 | ¹H NMR (400 MHz, CDCl₃) δ ppm 9.52 (1 H, br. s.), 6.95-7.06 (1 H, m), |
| | 6.81-6.88 (1 H, m), 5.91-6.04 (1 H, m), 5.62-5.77 (1 H, m), 5.36-5.49 (1 H, m), |
| | 5.15-5.29 (1 H, m), 4.97-5.11 (1 H, m), 4.25 (1 H, d), 4.02-4.16 (2 H, m), |
| | 3.42-3.62 (6 H, m), 3.41 (3 H, s), 3.31-3.35 (3 H, m), 3.25-3.31 (3 H, m), 2.98-3.14 (2 H, |
| | m), 2.82-2.98 (1 H, m), 2.61-2.80 (1 H, m), 2.45-2.60 (2 H, m), 1.62-1.77 (3 |
| | H, m), 1.21-1.36 (3 H, m). |

### BIOLOGICAL EXAMPLES

### Biological Example 1

### Antibacterial Activity Assay

Compounds were tested for antimicrobial activity against a panel of organisms according to standard procedures described by the National Committee for Clinical Laboratory Standards (Methods for Dilution Antimicrobial Susceptibility Tests for Bacteria that grow Aerobically 7^{th} edition Approved Standard M7-A7 Wayne Pa.: CLSI 2006) except that all testing was performed at 37° C. Compounds were dissolved in 100% DMSO and were diluted to the final reaction concentration (0.06 μg/mL-64 μg/mL) in microbial growth media. In all cases the final concentration of DMSO incubated with cells was less than or equal to 2.5%. For minimum inhibitory concentration (MIC) calculations, 2-fold dilutions of compounds were added to wells of a microtiter plate containing 5×10⁴ bacterial cells in a final volume of 200 μL of media (Mueller-Hinton Broth). The MIC value is defined as the lowest compound concentration inhibiting visible growth of the test organism. The MIC (in μg/mL) values of representative compounds of the present invention together with comparisons with the previously reported naturally occurring molecules are listed in Tables 3A-3C below. Whilst data against specific strains is reported herein, the specific strains selected are not critical for identifying active compounds. A person of skill in the art can readily select alternative or additional strains for MIC calculations.

Strains used:

S. aureus strains:

| | | |
|---|---|---|
| Sa1 | ATCC 13709 | Sensitive strain |
| Sa26 | ATCC 25923 | Sensitive strain |
| Sa4 | NCTC 6571 | Sensitive strain |

| | |
|---|---|
| Sa2 | Clinical strain registered under accession number NCIMB 41953 |
| | MRSA - Erythromycin Resistant - Fluoroquinolone resistant |

H. influenza strains

| | | |
|---|---|---|
| Hi3 | ATCC 31517 | Sensitive strain |
| Hi4 | Efflux pump KO | Sensitive strain |
| Hi106 | ATCC 51907 | Sensitive strain |
| Hi47 | Clinical strain | Ampicillin Resistant |
| Hi10 | Clinical strain | Sensitive strain |

E. coli strains:

| | | |
|---|---|---|
| Ec1 | ATCC 25922 | Sensitive strain |
| Ec50 | Efflux KO | Sensitive strain |
| EC113 | Efflux KO | Sensitive strain |

TABLE 3A
S. aureus

| | | | Sa26 + | Sa26 + | | |
|---|---|---|---|---|---|---|
| | Sa1 | Sa26 | 10% SH | 50% SH | Sa4 | Sa2 |
| Nargenicin A1 | 0.125 | 0.25 | 0.5 | 0.5 | <=0.06 | 0.125 |
| Nargenicin B1 | 1 | 2 | 2 | 1 | 0.5 | 0.5 |
| Nodusmicin | >64 | >64 | >64 | >64 | >64 | >64 |
| Branimycin | 64 | 64 | 64 | 64 | 64 | 64 |
| 1 | >64 | >64 | >64 | >64 | >64 | >64 |
| Baleomycin | | | | | | |
| 2 | 1 | 1 | 1 | 2 | 1 | 0.5 |
| 3 | 0.5 | 1 | 1 | 2 | 0.5 | 0.5 |
| 4 | 4 | 4 | 4 | 8 | 2 | 4 |
| 5 | 2 | 4 | 2 | 4 | 2 | 2 |
| 6 | >64 | >64 | >64 | >64 | >64 | 64 |
| 7 | 8 | 16 | 16 | 16 | 8 | 8 |
| 8 | >64 | >64 | >64 | >64 | >64 | >64 |
| 9 | 4 | 2 | 2 | 4 | 2 | 4 |
| 10 | 16 | 16 | 16 | 8 | 16 | 16 |
| 11 | >64 | >64 | >64 | >64 | >64 | >64 |
| 12 | 8 | 16 | 8 | 16 | 8 | 4 |
| 13 | >64 | >64 | >64 | >64 | 64 | >64 |
| 14 | >64 | >64 | >64 | >64 | >64 | >64 |
| 15 | >64 | >64 | >64 | >64 | >64 | >64 |
| 16 | 8 | 8 | 8 | 8 | 4 | 4 |
| 17 | >64 | >64 | >64 | >64 | >64 | >64 |
| 18 | 1 | 1 | 1 | 2 | 1 | 1 |
| 19 | 0.5 | 0.5 | 0.5 | 1 | 0.25 | 0.5 |
| 20 | 8 | 8 | 16 | 32 | 4 | 2 |
| 22 | 4 | 4 | 4 | 8 | 2 | 4 |
| 23 | 1 | 1 | 2 | 4 | 1 | 4 |
| 24 | 4 | 4 | 4 | 4 | 2 | 2 |
| 25 | 0.25 | 0.5 | 0.5 | 1 | 0.25 | <=0.06 |
| 26 | 64 | 128 | 128 | 64 | 64 | 64 |
| 27 | 1 | 1 | 2 | 4 | 1 | 1 |
| 28 | 2 | 4 | 4 | 16 | 4 | 4 |
| 29 | 0.5 | 1 | 1 | 2 | 0.5 | 0.5 |
| 30 | 16 | 16 | 32 | 64 | 16 | 8 |
| 31 | >64 | >64 | >64 | >64 | 64 | 64 |
| 32 | 4 | 16 | 16 | 64 | 8 | 4 |
| 33 | >64 | >64 | >64 | >64 | 64 | 64 |
| 34 | 64 | 64 | >64 | >64 | 32 | 64 |
| 35 | >64 | >64 | >64 | >64 | 64 | 64 |
| 36 | 64 | >64 | >64 | >64 | 32 | 64 |
| 37 | >64 | 64 | >64 | >64 | 64 | 64 |
| 38 | 32 | 64 | >64 | 64 | 16 | 32 |
| 39 | >64 | >64 | >64 | >64 | >64 | >64 |
| 40 | 0.25 | 1 | 1 | 1 | 0.12 | 0.25 |
| 41 | 4 | 2 | 4 | 32 | 2 | ND |
| 42 | 2 | 4 | 16 | 32 | 2 | 2 |

ND—not tested

TABLE 3B
H. influenza strains

| | | Hi4 | | | |
|---|---|---|---|---|---|
| | | efflux | | | |
| | Hi3 | pump KO | Hi10 | Hi47 | Hi106 |
| Nargenicin A1 | 64 | 0.5 | 32 | 64 | 32 |
| Nargenicin B1 | >64 | 8 | >64 | >64 | >64 |
| Nodusmicin | 64 | 2 | 64 | 32 | 64 |
| Branimycin | >64 | 4 | >64 | >64 | >64 |
| 1 | >64 | 2 | >64 | 64 | 64 |
| Baleomycin | | | | | |
| 2 | >64 | 4 | >64 | >64 | >64 |
| 3 | >64 | 1 | 64 | >64 | 64 |
| 4 | >64 | 16 | >64 | ND | ND |
| 5 | >64 | 8 | >64 | >64 | >64 |
| 6 | >64 | 32 | >64 | >64 | >64 |
| 7 | >64 | 0.5 | 64 | 64 | 64 |
| 8 | >64 | >64 | >64 | >64 | >64 |
| 9 | >64 | 16 | >64 | >64 | >64 |
| 10 | >64 | 32 | >64 | >64 | >64 |
| 11 | >64 | 32 | >64 | >64 | >64 |
| 12 | >64 | 4 | >64 | 64 | 64 |
| 13 | >64 | 16 | >64 | 64 | >64 |
| 14 | >64 | 64 | >64 | >64 | >64 |
| 15 | >64 | 64 | >64 | >64 | >64 |
| 16 | >64 | 16 | >64 | >64 | >64 |
| 17 | >64 | 8 | >64 | >64 | >64 |
| 18 | >64 | 4 | >64 | >64 | >64 |
| 19 | >64 | 0.25 | >64 | >64 | >64 |
| 20 | >64 | 32 | >64 | >64 | >64 |
| 22 | >64 | 32 | >64 | >64 | >64 |
| 23 | >64 | 0.5 | 64 | 64 | 64 |
| 24 | >64 | 8 | >64 | >64 | >64 |
| 25 | >64 | 1 | >64 | >64 | >64 |
| 26 | >128 | 8 | >128 | >128 | >128 |
| 27 | >64 | 8 | >64 | >64 | >64 |
| 28 | >64 | 4 | 64 | 64 | 64 |
| 29 | >64 | 2 | >64 | 64 | 64 |
| 30 | >64 | 4 | 64 | 64 | >64 |
| 31 | >64 | 16 | 16 | 32 | 32 |
| 32 | >64 | 4 | >64 | >64 | >64 |
| 33 | >64 | 16 | >64 | >64 | >64 |
| 34 | >64 | 8 | >64 | 16 | >64 |
| 35 | >64 | 16 | >64 | >64 | >64 |
| 36 | >64 | 32 | >64 | >64 | >64 |
| 37 | >64 | 16 | >64 | >64 | >64 |
| 38 | >64 | 16 | >64 | >64 | >64 |
| 39 | >64 | >64 | >64 | >64 | >64 |
| 40 | >64 | 2 | 64 | >64 | >64 |
| 41 | >64 | 8 | >64 | >64 | >64 |
| 42 | >64 | 4 | >64 | >64 | >64 |

ND—not tested

TABLE 3C
E. coli

| | | Ec50 | Ec113 |
|---|---|---|---|
| | | efflux | efflux |
| | Ec1 | pump KO | pump KO |
| Nargenicin A1 | >64 | 2 | <=0.06 |
| Nargenicin B1 | >64 | 16 | 2 |
| Nodusmicin | >64 | 1 | 0.25 |
| Branimycin | >64 | 8 | 1 |
| 1 | >64 | 2 | 0.5 |
| Baleomycin | | | |
| 2 | >64 | 16 | 1 |
| 3 | >64 | 8 | 0.125 |
| 4 | >64 | >64 | 4 |
| 5 | >64 | >64 | ND |
| 6 | >64 | ND | 8 |
| 7 | >64 | >64 | 0.125 |
| 8 | >64 | >64 | 64 |
| 9 | >64 | >64 | 4 |
| 10 | >64 | >64 | 16 |
| 11 | >64 | >64 | 8 |
| 12 | >64 | >64 | 2 |
| 13 | >64 | 64 | 8 |
| 14 | >64 | >64 | 16 |
| 15 | >64 | >64 | 16 |
| 16 | >64 | >64 | 4 |
| 17 | >64 | >64 | 8 |
| 18 | >64 | >64 | 2 |
| 19 | >64 | 64 | 0.5 |
| 20 | >64 | >64 | 16 |
| 22 | >64 | >64 | 8 |
| 23 | >64 | ND | 0.12 |
| 24 | >64 | 1 | 2 |
| 25 | >64 | 0.5 | 0.25 |
| 26 | >128 | 2 | 4 |
| 27 | >64 | >64 | 4 |
| 28 | >64 | 64 | 1 |
| 29 | >64 | 32 | 0.5 |
| 30 | >64 | 16 | 0.5 |
| 31 | >64 | >64 | 32 |
| 32 | >64 | >64 | 64 |
| 33 | >64 | >64 | 6 |
| 34 | >64 | >64 | 64 |
| 35 | >64 | >64 | 64 |
| 36 | >64 | >64 | 64 |
| 37 | >64 | >64 | 16 |
| 38 | >64 | >64 | 8 |
| 39 | >64 | >64 | >64 |
| 40 | >64 | 16 | 0.25 |
| 41 | >64 | 64 | 2 |
| 42 | >64 | 64 | 4 |

ND—not tested

### Biological Example 2

### In Vitro Activity Against DNA III Polymerase E

The replicative DNA polymerase III, a subunit, from Staphylococcus aureus (Biocat73824) was purified from a recombinant strain, containing a pBluePet-DnaE(AA1-1022) construct.

The E. coli strain BL21(DE3) was transformed with the pBluePet-DnaE(AA1-1022) construct and was grown in Terrific Broth (TB) medium under 220 rpm shaking at 37° C. Terrific Broth was prepared by following procedure: tryptone peptone ((12 g, DIFCO, #211705), Bacto™ yeast extract (24 g, BD, #212750) and glycerol (4 mL) were added to water (900 mL final volume), sterilized and the volume was adjusted to 1000 mL by addition of 100 mL of KH₂PO₄ (170 mM) and K₂HPO₄ (720 mM) stock solution. When OD₆₀₀ reached 0.5, bacteria were induced with 1 mM isopropyl β-D-1-thiogalactopyranoside and left at 25° C. for 14-16 hours under 220 rpm shaking. Cells were harvested by centrifugation at 6,000×g and frozen at −20° C. before use. Expression yield was determined after lysis using B-PER® Bacterial Protein Extraction Reagent (Thermo Scientific, #78248) as described by the manufacturer, giving 100 mg/L of soluble protein.

A total of 16 g (wet weight) of E. coli BL21(DE3) paste was suspended in 11 volumes of lysis buffer (50 mM Tris pH 8, 50 mM NaCl, 10% glycerol 100 mM lysozyme and protease cocktail inhibitor (Roche Diagnostics, #11873580001)). The pellet was homogenized at 4° C. by magnetic stirring for 15 min. Cells were broken by sonication (150 pulses of 4 sec (8 sec off) using a 13 mm diameter probe, in icy bath). To hydrolyze DNA and RNA, 250 U/mL of Benzonase® nuclease (Novagen, #70746-3) was added before ultracentrifugation at 142,400×g for one hour at 4° C. Supernatant was recovered and loaded on a 5 mL Histrap™ column HP (GE Healthcare, 17-5248-02) preequilibrated in buffer A (50 mM Tris pH 8, 20 mM NaCl, 10% glycerol, 10 mM (3-Mercaptoethanol). The column was first washed with 10 column-volumes of buffer A +1.3 M NaCl then with 10 column-volumes of buffer A +30 mM Imidazole to remove unspecific binding. Bound proteins were eluted with a 10-column-volume linear gradient of buffer B (50 mM Tris pH 8, 20 mM NaCl, 10% glycerol, 10 mM β-Mercaptoethanol, 500 mM imidazole). Fractions containing DnaE from Staphylococcus aureus, as determined by SDS-PAGE analysis, were pooled giving 70 mg of 95% pure target protein (final yield: 87.5 mg/L).

A radioactive filterplate assay was used to assess inhibitory activity of compounds on DNA Polymerase IIIα. Five μL of a dilution series of compound, starting from 100 μM highest concentration, 1/5 dilution, was added to the wells of a 96 well plate. Recombinant enzyme was diluted to 0.47 μg/mL in a buffer containing 20 mM Tris pH7.5, 8 mM DTT, 10 mM MgOAc, 0.05% CHAPS and 10 μL thereof was added to the compound dilutions. The reaction was started with the addition of 10 μL substrate in the same buffer, containing activated calf thymus DNA (Sigma, D4522), dATP, dGTP, dCTP, dTTP (Invitrogen) and [α-33P]-dTTP (Perkin Elmer, NEG605H001) at a concentration of 62.5 μg/mL, 50 nM and 7.5 μCi/mL respectively. The mixture was incubated at 30° C. for 120 minutes and terminated by addition of phosphoric acid. Samples were transferred to filter plates and incorporated radioactivity was measured by the Topcount. Data were converted to percent inhibition with respect to positive and negative controls. IC₅₀ values were calculated using Graph Prism™ software.

### Semiquantitative Scoring:

| | S. aureus | E. coli |
|---|---|---|
| Nargenicin A1 | *** | ### |
| Nargenicin B1 | *** | # |
| Nodusmicin | *** | ## |
| Branimycin | *** | ## |
| 1 | ** | ## |
| Baleomycin | | |
| 2 | *** | ## |
| 3 | *** | ## |
| 4 | *** | # |
| 5 | *** | # |
| 6 | *** | # |
| 7 | *** | ### |
| 8 | ** | # |
| 9 | *** | # |
| 10 | *** | # |
| 11 | ** | # |
| 12 | *** | # |
| 13 | ** | # |
| 14 | ** | # |
| 15 | ** | # |
| 16 | *** | # |
| 17 | *** | # |
| 18 | *** | ## |
| 19 | *** | ## |
| 20 | *** | # |
| 21 | ND | ND |
| 22 | *** | # |
| 23 | *** | ## |
| 24 | *** | # |
| 25 | *** | ## |
| 26 | *** | ## |
| 27 | *** | ND |
| 28 | *** | ## |
| 29 | *** | ND |
| 30 | ND | ND |
| 31 | ND | ND |
| 32 | *** | ND |
| 33 | ** | ND |
| 34 | ** | ND |
| 35 | ** | ND |
| 36 | *** | ND |
| 37 | *** | ND |
| 38 | ND | ND |
| 39 | ** | ND |
| 40 | *** | ### |
| 41 | *** | ND |
| 42 | ND | ND |

ND—not tested

| | | |
|---|---|---|
| | S. aureus | E. coli |
| 0.001-1 μg/mL | *** | ### |
| 1.0-10 μg/mL | ** | ## |
| >10 μg/mL | * | # |

### Biological Example 3

### In Vivo Efficacy

The compounds of the invention and comparative examples from the parent molecules were tested in several in vivo models of infection. In particular data from infection with S. aureus in thigh, lung and groin is presented herein.

### 3.1 Thigh Model of Infection

The neutropenic mouse thigh infection model is well known and has been used extensively for determination of pharmacokinetic/pharmacodynamic (PK/PD) index determination and prediction of antibiotic efficacy in patients since its description by W. A. Craig, J. Redington, and S. C. Ebert, J. Antimicrob. Chemother. 27[Suppl. C]:29-40, 1991.

The in vivo antibacterial activity was established by infecting both thighs of male CD-1 mice (Charles River Lab, Lyon, France) weighing 19-23 g with Methicillin Resistant S. aureus (MRSA) inoculum. Before infection, mice were rendered neutropenic (neutrophil, <100/mm³) by injecting them with cyclophosphamide (SIGMA, St Louis) intraperitoneally 4 days (150 mg/kg of body weight) and 1 day (100 mg/kg) before thigh infection. The inoculum was prepared from Methicillin Resistant clinical isolate of S. aureus. The optical density of a broth culture of freshly plated bacteria was adjusted to 0.1 at an absorbance at 590 nm, then put at 37° C. in shaken culture for about 1 h30 until a OD=0.3 at 590 nm. After a 1/10,000 dilution into physiological saline, 0.1 mL of this inoculum suspension was injected into each thigh.

The test compound was dissolved in methyl cellulose at 0.5% (for oral treatment) or polyethyleneglycol 200 at 20% (for parenteral treatments) to give a solution of 2 mg/mL (pH=7.0). This solution was diluted with vehicle to give 0.6 mg/mL solutions. One hour post infection (PI), animals were treated orally (po) or via parenteral routes (ip, sc or iv) as indicated in the experimental tables. A group of untreated mice received only the corresponding vehicle.

Administration was repeated seven hours post-infection (PI) for the twice a day (BID) model or at 24 h PI for the once a day (QD) model. Twenty four hours PI for the BID model or 48 h PI for the QD model, all mice were euthanized, each thigh was removed, and the bacterial burden in the thigh muscles was enumerated after tissue homogenization and plating.

The results of the in vivo efficacy test are summarized in Table 5A, which provides a representative example of the results obtained for Compounds 2 and 19.

In this model, Ciprofloxacin is used as the negative control, and Vancomycin (parenteral) and/or Linezolid (parenteral or oral) is used as the positive control. A test compound is considered active when it shows a log reduction equivalent to the positive control in the same study.

TABLE 5A
Thigh Sa - (Nd of treatment = 2)

| | | | | | | Log | |
|---|---|---|---|---|---|---|---|
| | | Dose | | | Sample | reduction vs. | |
| Study | Compound | mg/kg | Route | Frequency | time, PI | untreated | Active |
| 1 | Vancomycin | 50 | SC | BID | 7 h | −4.01 | Y |
| | Ciprofloxacin | 50 | SC | | | −1.27 | N |
| | Branimycin | 100 | IP | | | −0.3 | N |
| 2 | Vancomycin | 50 | SC | BID | 7 h | −2.57 | Y |
| | Ciprofloxacin | | SC | | | −1.07 | N |
| | Compound 2 | | IP | | | −3.55 | Y |
| 3 | Vancomycin | 50 | SC | BID | 7 h | −1.52 | Y |
| | Ciprofloxacin | | SC | | | 0.22 | N |
| | Nargenicin A1 | | SC | | | −4.52 | Y |
| 4 | Vancomycin | 15 | SC | BID | 24 h | −3.96 | Y |
| | Ciprofloxacin | 50 | SC | | | −0.05 | N |
| | Compound 2 | 5 | IP | | | −1.16 | N |
| | | 15 | | | | −3.32 | Y |
| | | 50 | | | | −4.74 | Y |
| 5 | Ciprofloxacin | 50 | SC | BID | 24 h | −0.27 | N |
| | Vancomycin | 50 | SC | | | −4.27 | Y |
| | Linezolid | 50 | PO | | | −4.07 | Y |
| | | 50 | SC | | | −4.64 | Y |
| | Nargenicin A1 | 5 | IP | | | 0.28 | N |
| | | 15 | IP | | | −0.55 | N |
| | | 50 | IP | | | −5.40 | Y |
| | | 200 | PO | | | −4.47 | Y |
| | | 50 | SC | | | −3.28 | Y |
| | Compound 2 | 5 | IV | | | −0.13 | N |
| 6 | Ciprofloxacin | 50 | SC | BID | 24 h | −0.26 | N |
| | Linezolid | 25 | PO | | | −4.54 | Y |
| | Linezolid | 50 | PO | | | −3.49 | Y |
| | Nargenicin A1 | 50 | PO | | | −2.02 | Y |
| | | 100 | PO | | | −4.06 | Y |
| | Compound 2 | 25 | IV | | | −3.92 | Y |
| 7 | Ciprofloxacin | 50 | SC | BID | 24 h | −0.08 | N |
| | Linezolid | 15 | PO | | | −2.98 | Y |
| | Linezolid | 50 | PO | | | −5.13 | Y |
| | Compound 2 | 15 | PO | | | −4.13 | Y |
| | | 50 | PO | | | −6.72 | Y |
| 8 | Ciprofloxacin | 50 | SC | QD | 48 h | −0.39 | N |
| | Vancomycin | 50 | SC | | | −3.2 | Y |
| | Linezolid | 50 | PO | | | −2.78 | Y |
| | Compound 2 | 50 | PO | | | −4.63 | Y |
| 9 | Ciprofloxacin | 50 | SC | BID | 24 h | −1.28 | N |
| | Linezolid | 50 | PO | | | −4.01 | Y |
| | Compound 19 | 50 | PO | | | −3.28 | Y |

### 3.2 Lung Model of Infection

To see if compounds are potentially efficacious to treat pneumonia, they were tested in a lung infection model in mice induced by a methicillin resistant S. aureus clinical isolate Sa2.

The in vivo antibacterial activity was established by infecting lungs of male CBAJ mice (Charles River Lab, Lyon, France) weighing 19-23 g with Methicillin Resistant S. aureus Sa2 (MRSA) inoculum. Before infection, mice were rendered neutropenic (neutrophil, <100/mm³) by injecting them with cyclophosphamide (SIGMA, St Louis) intraperitoneally 4 days (150 mg/kg of body weight) and 1 day (100 mg/kg) before thigh infection. The inoculum was prepared from Methicillin Resistant clinical isolate of S. aureus Sa2. The optical density of a broth culture of freshly plated bacteria was adjusted to 0.1 at an absorbance at 590 nm, then put at 37° C. in shaken culture for about 1 h30 until a OD=0.3 at 590 nm. After a 1/50 dilution into physiological saline, 0.05 mL of this inoculum suspension was instilled intranasally.

The test compound was dissolved in methyl cellulose at 0.5% to give a solution of 2 mg/ml (pH=7.0). This solution was diluted with vehicle to give 1, 0.6, and 0.2 mg/mL solutions. Two hour post infection (PI), animals were treated orally, compounds were diluted in methyl cellulose at 0.5%, a group of untreated mice was administered only the vehicle. Administration was repeated twenty hours post-infection (PI) for the twice a day (BID) model or 24 h and 48 h PI for the once a day (QD) model. Twenty four hours PI for the BID model, or 72 h PI for the QD all mice were euthanized, each lung was removed and the bacterial burden in the lungs enumerated after tissue homogenization and plating.

The results of the in vivo efficacy test are summarized in Table 5B, which provides a representative example of the results obtained for Compounds 2, 19, 25 and 28.

In this model, Levofloxacin or Ciprofloxacin is used as the negative control, and Vancomycin (parenteral or oral) and/or Linezolid (oral) is used as the positive control. A test compound is considered active when it shows a log reduction equivalent to the positive control in the same study.

TABLE 5B
Lung Sa (Nd of Treatment = 2)

| | | Tested | | | Sample | Log | |
|---|---|---|---|---|---|---|---|
| | | dose | | | time, post | reduction vs. | |
| Study | Compound | mg/kg | Route | Frequency | infection | untreated | Active |
| 1 | Vancomycin | 50 | SC | BID | 24 h | −2.00 | Y |
| | Levofloxacin | | PO | | | −0.32 | N |
| | Linezolid | | PO | | | −2.92 | Y |
| | Compound 2 | | PO | | | −3.36 | Y |
| | Compound 25 | | PO | | | −0.36 | N |
| 2 | Levofloxacin | 50 | PO | BID | 24 h | −0.55 | N |
| | Linezolid | 15 | | | | −3.29 | Y |
| | Linezolid | 50 | | | | −4.64 | Y |
| | Compound 2 | 15 | | | | −3.91 | Y |
| | | 50 | | | | −2.99 | Y |
| 3 | Levofloxacin | 50 | PO | BID | 24 h | 1.17 | N |
| | Linezolid | 50 | | | | −2.70 | Y |
| | Compound 19 | 50 | | | | −3.56 | Y |
| | Compound 28 | 50 | | | | −1.64 | N |
| 4 | Linezolid | 25 | PO | BID | 24 h | −3.10 | Y |
| | Cipro | 50 | | | | −0.82 | N |
| | Nargenicin A1 | 12.5 | | | | −0.39 | N |
| | | 25 | | | | −2.17 | Y |
| | | 50 | | | | −2.57 | Y |
| | Compound 2 | 6.25 | | | | −0.04 | N |
| | | 12.5 | | | | −1.9 | Y |
| | | 25 | | | | −3.48 | Y |
| 5 | Linezolid | 5 | PO | QD | 72 h | −0.37 | N |
| | | 15 | | | | −1.49 | Y |
| | Ciprofloxacin | 50 | | | | −0.91 | N |
| | Compound 2 | 5 | | | | −1.57 | Y |
| | | 15 | | | | −3.62 | Y |
| 6 | Linezolid | 25 | PO | QD | 72 h | −2.37 | Y |
| | Ciprofloxacin | | | | | −0.44 | N |
| | Compound 2 | | | | | −3.65 | Y |
| | Compound 19 | | | | | −3.65 | Y |

### 3.3 Skin Model of Infection (Groin)

To see if compounds are potentially efficacious to treat skin infections, they were tested in an abscess model in mice induced by a methicillin resistant S. aureus clinical isolate Sa2.

The in vivo antibacterial activity was established by infecting the groin of male CD1 mice (Charles River Lab, Lyon, France) weighing 19-23 g with Methicillin Resistant S. aureus Sa2 (MRSA) inoculum. The inoculum was prepared from Methicillin Resistant clinical isolate of S. aureus Sa2. An overnight culture of the strain was diluted 1/10,000 in physiological water and then 0.5 mL was injected subcutaneously into the groin.

The test compound was dissolved in methyl cellulose at 0.5% or polyethylene glycol 200 at 20% (depending on administration route) to give a solution of 2 mg/mL (pH=7.0). Two hour post infection (PI), animals were treated orally or by parenteral routes (ip or sc) depending on experiment, a group of untreated mice was administrated only with the corresponding vehicle. Administration was repeated seven hours post-infection (PI) and 24 h PI. Thirty one hours PI, all mice were euthanized, each groin was removed and the bacterial burden in the lungs enumerated after tissue homogenization and plating.

The results of the in vivo efficacy test are summarized in Table 5C, which provides a representative example of the results obtained for Compound 2.

In this model, Ciprofloxacin or Levofloxacin is used as the negative control, and Vancomycin (parenteral) and/or Linezolid (oral) is used as the positive control. A test compound is considered active when it shows a log reduction equivalent to the positive control in the same study.

TABLE 5C
Groin Sa (Nd of Treatment = 3)

| | | Tested | | | Sample | Log | |
|---|---|---|---|---|---|---|---|
| | | dose | | | time, post | reduction vs. | |
| Study | Compound | mg/kg | Route | Frequency | infection | untreated | Active |
| 1 | Ciprofloxacin | 50 | SC | BID | 31 h | −1.57 | N |
| | Vancomycin | | SC | | | −3.59 | Y |
| | Nargenicin A1 | | PO | | | −1.11 | N |
| | | | IP | | | −3.13 | Y |
| 2 | Levofloxacin | 50 | PO | BID | 31 h | −1.00 | N |
| | Linezolid | | | | | −2.44 | Y |
| | Compound 2 | | | | | −2.52 | Y |

1. A compound according to Formula I: 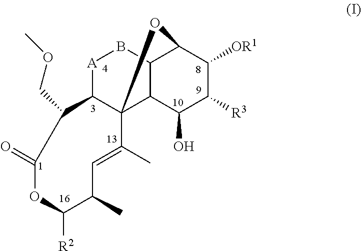
wherein,
A and B together form a bivalent radical —CH═CH— or —CH₂—CH₂—;
R¹ is H or —C(═O)—R⁴,
R⁴ is a 5-membered heteroaryl containing 1 or 2 heteroatoms selected from O, S and N, optionally substituted by one or more CH₃, halogen, or CN;
R² is H or CR^{2a}R^{2b}R^{2c},
R^{2a} is selected from H, OH, and OCH₃,
R^{2b} is H or CH₃, or
R^{2a} and R^{2b} together form oxo or ═N—OR⁵, wherein R⁵ is H, CH₃, or —C(═O)CH₃,
R^{2c} is CH₂—O—CH₃; and
R³ is CH₃ or CH₂—O—CH₃;
with the proviso that when A and B together form a bivalent radical —CH═CH—, R² is CR^{2a}R^{2b}R^{2c}, where R^{2a} is OH, R^{2b} is H and R^{2c} is CH₂—O—CH₃, and R³ is CH₂—O—CH₃, then R¹ is —C(═O)—R⁴;
or a salt thereof.

2. The compound or a salt thereof according to claim 1, wherein R¹ is —C(O)R⁴.

3. The compound or a salt thereof according to claim 2, wherein R⁴ is selected from: 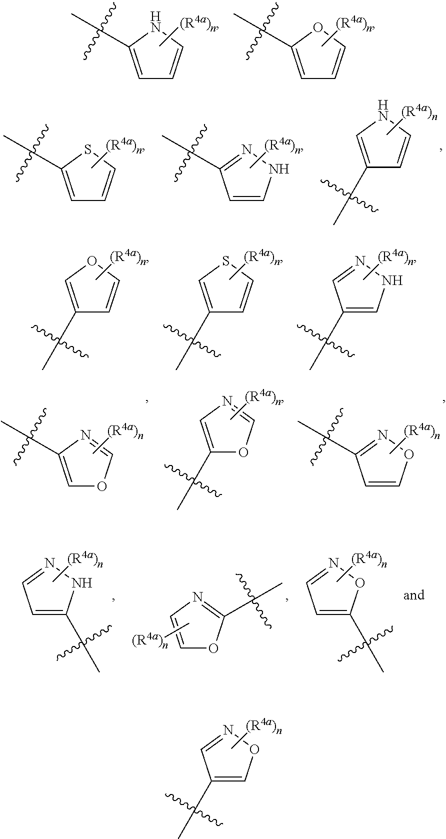
wherein R^{4a} is selected from H, CN, CH₃ and halogen, and integer n is 1 or 2.

4. The compound or a salt thereof according to claim 3, wherein R⁴ is selected from: 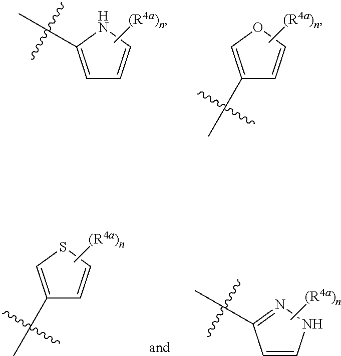
wherein R^{4a} is selected from H, CN, CH₃ and halogen, and integer n is 1 or 2.

5. The compound or a salt thereof according to claim 2, wherein R⁴ is selected from: 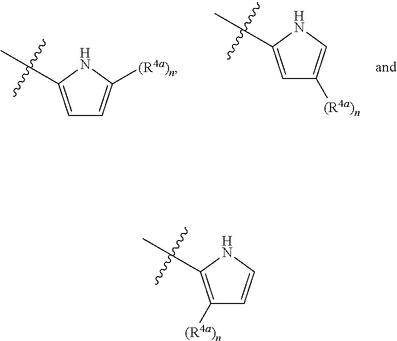
wherein R^{4a} is selected from H, CN, CH₃, F and Cl, and integer n is 1.

6. The compound or a salt thereof according to claim 4, wherein R⁴ is: 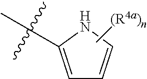
wherein R^{4a} is selected from H, CN, CH₃, F and Cl, and integer n is 1.

7. The compound or a salt thereof according to claim 1, wherein R² is CR^{2a}R^{2b}R^{2c}.

8. The compound or a salt thereof according to claim 7, wherein R^{2a} is OH, R^{2b} is H and R^{2C} is CH₂—O—CH₃.

9. The compound or a salt thereof according to claim 7, wherein R^{2a} and R^{2b} together form oxo or ═N—OR⁵.

10. The compound or a salt thereof according to claim 1, wherein the salt is a pharmaceutically acceptable salt.

11. A pharmaceutical composition comprising a pharmaceutically acceptable carrier and a pharmaceutically effective amount of a compound according to claim 1.

12. The pharmaceutical composition according to claim 11 comprising a further therapeutic agent.

13. (canceled)

14. (canceled)

15. A method for the treatment, or prevention of bacterial infectious diseases comprising administering an amount of a compound according to claim 1 sufficient to effect said treatment, or prevention.

16. The method according to claim 15, wherein the compound is administered in combination with a further therapeutic agent.

17. A method for the treatment, or prevention of bacterial infectious diseases comprising administering an amount of a pharmaceutical composition of claim 11, sufficient to effect said treatment, or prevention.

18. The method according to claim 17, wherein the pharmaceutical composition is administered in combination with a further therapeutic agent.
